(19) United States
(12) Patent Application Publication
(10) Pub. No.: US 2026/0308587 A1
(43) Pub. Date: Oct. 8, 2026

(54) Sugar Modified Oligonucleotides and Uses Thereof

(71) Applicant: Ionis Pharmaceuticals, Inc., Carlsbad, CA (US)

(72) Inventors: Mehran Nikan, Oceanside, CA (US); Thazha P. Prakash, Carlsbad, CA (US); Eric E. Swayze, Encinitas, CA (US)

(73) Assignee: Ionis Pharmaceuticals, Inc., Carlsbad, CA (US)

(21) Appl. No.: 19/480,561

(22) PCT Filed: May 3, 2024
(86) PCT No.: PCT/US2024/027714

§ 371 Date: Oct. 31, 2025

Related U.S. Application Data
(60) Provisional application No. 63/499,920, filed on May 3, 2023.

(51) Int. Cl. C12N 15/113

(52) U.S. Cl. CPC C12N 15/113; C12N 2310/14; C12N 2310/312; C12N 2310/315; C12N 2310/321; C12N 2310/322; C12N 2310/531

(57) ABSTRACT

The present disclosure provides RNAi agents comprising a stereo-non-standard nucleoside in the seed region thereof.

### SEQUENCE LISTING

The present application is being filed along with a Sequence Listing in electronic format. The Sequence Listing is provided as a file entitled CHEM0109WOSEQ.xml created May 2, 2024, which is 27 kb in size. The information in the electronic format of the sequence listing is incorporated herein by reference in its entirety.

### FIELD

The present disclosure provides RNAi agents comprising at least one modified oligonucleotide having in the seed region at least one stereo-non-standard nucleoside.

### BACKGROUND

The principle behind antisense technology is that an antisense compound hybridizes to a target nucleic acid and modulates the amount, activity, and/or function of the target nucleic acid. For example, in certain instances, antisense compounds result in altered transcription or translation of a target. Such modulation of expression can be achieved by, for example, target RNA degradation or occupancy-based inhibition. An example of modulation of RNA target function by degradation is RNase H-based degradation of the target RNA upon hybridization with a DNA-like antisense compound.

Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Another example of modulation of gene expression is the use of antisense compounds in a CRISPR system. Regardless of the specific mechanism, sequence-specificity makes antisense compounds attractive as tools for target validation and gene functionalization, as well as therapeutics to selectively modulate the expression of genes involved in the pathogenesis of disease.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, tolerability, pharmacokinetics, or affinity for a target nucleic acid. While RNAi antisense-mediated gene silencing is effective, there is a risk of off-target effects. Thus, there is a need for optimized chemistry designs for use in RNAi therapeutics that can reduce such off-target effects.

### SUMMARY

The present disclosure provides an antisense RNAi oligonucleotide comprising modified oligonucleotides consisting of linked nucleosides linked through internucleoside linking groups, wherein at least one of the nucleosides comprises a stereo-non-standard nucleoside. In certain embodiments, the antisense RNAi oligonucleotide may comprise a stereo-non-standard nucleoside in the seed region thereof, wherein the stereo-non-standard nucleoside comprises a sugar moiety such as a 2′-deoxy, a 2′-OH, or a 2′-modified sugar moiety selected from those having certain stereochemical configurations as described herein, for example, with respect to Formula X. In certain embodiments, the 2′-substituent may be a 2′-F or a 2′-OMe. Such stereo-non-standard nucleosides described herein may provide seed-region destabilization of RNA interference (RISC) complexes. In certain embodiments, the stereo-non-standard nucleosides described herein may increase selectivity of RNA interference when compared to an analogous RNAi oligonucleotide (e.g., one with the same motif or pattern of 2′-substituents) that includes only stereo-standard nucleosides in the seed region.

Also provided are methods of administering the RNAi agents to a subject, for example to a mammal such as a human.

In certain embodiments, RNAi agents of the instant disclosure provide a favorable off-target profile compared to an analogous RNAi agent not including a stereo-non-standard nucleoside in the seed region thereof. For example, reduced off-target effect may be determined by IC50 for knockdown of one or more off-target genes. For example, reduced off-target effect may be determined by number of differentially expressed off-target genes.

In some embodiments, the stereo-non-standard nucleoside has a structure of Formula X:

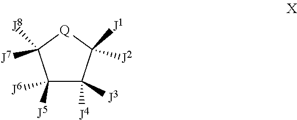

wherein one of J¹ and J² is H and the other is a heterocyclic base moiety Bx;
one of J³ and J⁴ is H and the other is a 2′-substituent selected from H, OH, OMe, F, and OCH₂CH₂OCH₃;
one of J⁵ and J⁶ is H and the other is —O-5′ linkage;
one of J⁷ and J⁸ is H and the other is —C(R¹)₂O-3′ linkage;
   wherein each R¹ is independently H or C₁₋₆ alkyl;
provided that when J² and J⁸ are both H, then one of J³ and J⁵ is not H:
Q is O, S, or NR²;
   provided that when Q is O and J² is H, then J^{P} is not H;
   and
wherein R² is H, C₁₋₆ alkyl, or acetyl.

In certain embodiments of Formula X, the heterocyclic base moiety Bx is selected from uracil, thymine, cytosine, 5-methyl cytosine, adenine and guanine. In certain embodiments, the heterocyclic base moiety Bx is a modified base as described herein or as known in the art.

In some embodiments, the stereo-non-standard nucleoside has a structure selected from Formula 1. Formula II, Formula III, Formula IV, Formula V, Formula VI, and Formula VII:

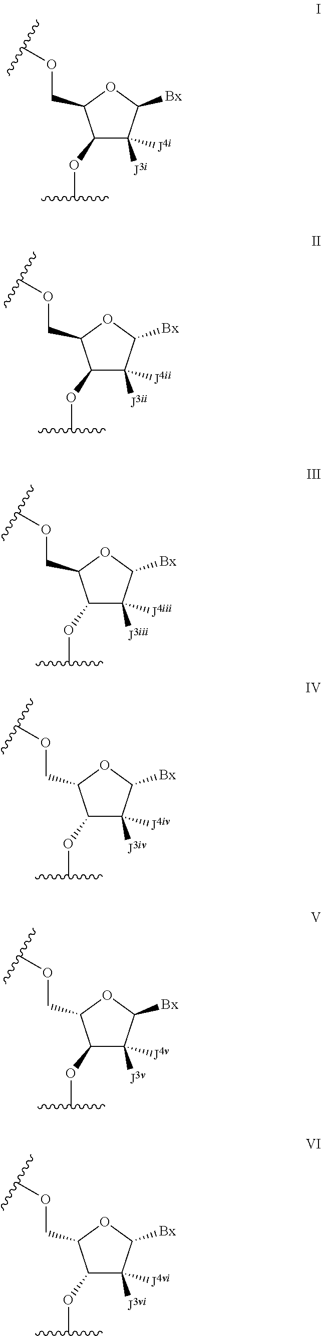

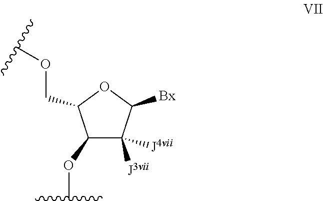

wherein J³ⁱ, J³ⁱⁱ, J³ⁱⁱⁱ, J^{3iv}, J^{3v}, J^{3vi} and J^{3vii} are as defined for J³ in Formula X; and J⁴ⁱ, J⁴ⁱⁱ, J⁴ⁱⁱⁱ, J^{4iv}, J^{4v}, J^{4vi}, and J^{4vii} are as defined for J⁴ in Formula X. and
Bx is a is a heterocyclic base moiety.

In certain embodiments, the stereo-non-standard nucleoside has a structure of Formula 1 in which J³ⁱ and J⁴ⁱ are each H.

In some embodiments, provided is composition comprising an RNAi agent and a pharmaceutically acceptable carrier or diluent, wherein the RNAi agent comprises an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, wherein the antisense RNAi oligonucleotide comprises within its seed region at least one stereo non-standard nucleoside having Formula X:

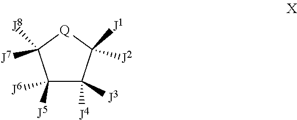

wherein one of J¹ and J² is H and the other is a heterocyclic base moiety Bx;
one of J³ and J⁴ is H and the other is a 2′-substituent selected from H, OH, OMe, F, and OCH₂CH₂OCH₃;
one of J⁵ and J⁶ is H and the other is —O-5′ linkage;
one of J⁷ and J⁸ is H and the other is —C(R¹)₂O-3′ linkage;
   wherein each R¹ is independently H or C₁₋₆ alkyl;
provided that when J² and J⁸ are both H, then one of J³ and J⁵ is not H;
Q is O, S, or NR²; and
   wherein R² is H, C₁₋₆ alkyl, or acetyl.

In further embodiments, provided is a method comprising administering to a subject in need thereof a composition comprising an RNAi agent and a pharmaceutically acceptable carrier or diluent, optionally in a therapeutically effective amount thereof, wherein the RNAi agent comprises an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, wherein the antisense RNAi oligonucleotide comprises within its seed region at least one stereo non-standard nucleoside having Formula X:

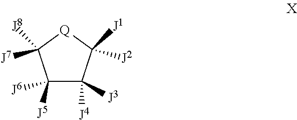

wherein one of J¹ and J² is H and the other is a heterocyclic base moiety Bx;
one of J³ and J⁴ is H and the other is a 2′-substituent selected from H, OH, OMe, F, and OCH₂CH₂OCH₃;
one of J⁵ and J⁶ is H and the other is —O-5′ linkage;
one of J⁷ and J⁸ is H and the other is —C(R¹)₂O-3′ linkage;
   wherein each R¹ is independently H or C₁₋₆ alkyl;
provided that when J² and J⁸ are both H, then one of J³ and J⁵ is not H;
Q is O, S, or NR²; and
   wherein R² is H, C₁₋₆ alkyl, or acetyl.

In certain embodiments, the antisense RNAi oligonucleotide includes a single stereo-non-standard nucleoside having a structure selected from Formula X, Formula I, Formula II, Formula III, Formula IV, Formula V, Formula VI, and Formula VII. In certain embodiments, the remainder of the nucleosides in the antisense RNAi oligonucleotide are stereo-standard nucleosides. In certain embodiments, the remainder of the nucleosides in the RNAi agent are stereo-standard nucleosides.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

Herein, compounds described as having “the nucleobase sequence of” a SEQ ID, unless otherwise indicated, include such compounds wherein each nucleobase is independently modified or unmodified, independent of nucleobase modifications, or absence of nucleobase modifications, indicated in the refenced SEQ ID. Further, such description of compounds by reference to a SEQ ID does not limit sugar or internucleoside linkage modifications, which, unless otherwise indicated, are independent of nucleobase sequence and nucleobase modification.

It is understood that the nucleobase sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH(H) sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, a modified oligonucleotide having the nucleobase sequence “ATCGATCG” encompasses any modified oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and modified oligonucleotides having other modified nucleobases, such as “AT^{m}CGAUCG,” wherein ^{m}C indicates a cytosine base comprising a methyl group at the 5-position. One of skill in the art will readily appreciate that labeling such nucleic acid compounds “RNA” or “DNA” does not alter or limit such nucleic acid compounds. While effort has been made to accurately describe compounds in the accompanying ST.26 compliant sequence listing, should there be any discrepancies between a description in this specification and in the accompanying sequence listing, the description in the specification controls.

As used herein, “2′-substituted” in reference to a furanosyl sugar moiety or nucleoside comprising a furanosyl sugar moiety means the furanosyl sugar moiety or nucleoside comprising the furanosyl sugar moiety comprises a substituent other than H,H at the 2′-position, and is a not a bicyclic furanosyl sugar moiety.

As used herein, “administration” or “administering” refers to routes of introducing a compound or composition provided herein to a subject to perform its intended function. Examples of routes of administration that can be used include, but are not limited to, administration by inhalation, subcutaneous injection, intrathecal injection, and oral administration.

As used herein, “antisense activity” means any detectable and/or measurable change attributable to the hybridization of an antisense oligonucleotide to its target nucleic acid. In certain embodiments, antisense activity is a decrease in the amount or expression of a target nucleic acid or protein encoded by such target nucleic acid compared to target nucleic acid levels or target protein levels in the absence of the antisense oligonucleotide.

As used herein, “antisense agent” means an antisense oligonucleotide or an oligomeric duplex comprising an antisense oligonucleotide.

As used herein, “antisense compound” means an antisense oligonucleotide or an oligomeric duplex comprising an antisense oligonucleotide.

As used herein, “antisense oligonucleotide” means an oligonucleotide that is complementary to a target nucleic acid and is capable of achieving at least one antisense activity. Antisense oligonucleotides include but are not limited to antisense RNAi modified oligonucleotides and RNase H antisense modified oligonucleotides. In certain embodiments, an antisense oligonucleotide is paired with a sense oligonucleotide to form an oligomeric duplex. In certain embodiments, an antisense oligonucleotide is unpaired and is a single-stranded antisense oligonucleotide. In certain embodiments, an antisense oligonucleotide comprises a conjugate group.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety, and the bicyclic sugar moiety is a modified bicyclic furanosyl sugar moiety. In certain embodiments, the bicyclic sugar moiety does not comprise a furanosyl moiety.

As used herein, “cEt” or “constrained ethyl” or “cEt sugar moiety” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH₃)—O-2′, and the methyl group of the bridge is in the S configuration. A cEt bicyclic sugar moiety is in the β-D configuration.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T), adenine (A) and uracil (U), cytosine (C) and guanine (G). 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms consisting of a conjugate moiety and a conjugate linker.

As used herein, “conjugate moiety” means a group of atoms that modifies one or more properties of a molecule compared to the identical molecule lacking the conjugate moiety, including but not limited to phanmacodynamics, pharmacokinetics, stability, binding, absorption, tissue distribution, cellular distribution, cellular uptake, charge and clearance.

As used herein, “conjugate linker” means a group of atoms comprising at least one bond.

As used herein, “expression” includes all the functions by which a gene's coded information is converted into structures present and operating in a cell. Such structures include, but are not limited to, the products of transcription and translation. As used herein, “modulation of expression” means any change in amount or activity of a product of transcription or translation of a gene. Such a change may be an increase or a reduction of any amount relative to the expression level prior to the modulation.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression or activity in an untreated or control sample and does not necessarily indicate a total elimination of expression or activity.

As used herein, “internucleoside linkage” or “internucleoside linking group” means a group or bond that forms a covalent linkage between adjacent nucleosides in an oligonucleotide. As used herein “modified internucleoside linkage” means any internucleoside linkage other than a naturally occurring, phosphodiester internucleoside linkage. “Phosphorothioate linkage” means a modified internucleoside linkage in which one of the non-bridging oxygen atoms of a phosphodiester is replaced with a sulfur atom. Modified interucleoside linkages may or may not contain a phosphorus atom. A “neutral internucleoside linkage” is a modified internucleoside linkage that does not have a negatively charged phosphate in a buffered aqueous solution at pH=7.0. A modified internucleoside linkage may optionally comprise a conjugate group.

As used herein, “linked nucleosides” are nucleosides that are connected in a continuous sequence (i.e. no additional nucleosides are present between those that are linked).

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein. “modulating” refers to changing or adjusting a feature in a cell, tissue, organ or organism.

As used herein, “2′-deoxynucleoside” means a nucleoside according to the structure:

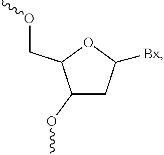

wherein Bx is a nucleobase.

A 2′-deoxynucleoside may be a stereo-standard nucleoside or a stereo-non-standard nucleoside.

As used herein, “2′-deoxy sugar moiety” means the sugar moiety of a 2′-deoxynucleoside.

As used herein, “2′-MOE nucleoside” means a nucleoside according to the structure:

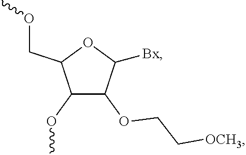

wherein Bx is a nucleobase.

As used herein, “2′-MOE sugar moiety” means the sugar moiety of a 2′-MOE nucleoside as defined herein.

“MOE” means an —OCH₂CH₂OCH₃ group.

A 2′-MOE nucleoside may be a stereo-standard nucleoside or a stereo-non-standard nucleoside.

As used herein, “2′-OMe nucleoside” means a nucleoside according to the structure:

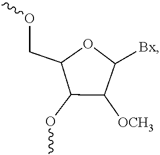

wherein Bx is a nucleobase.

A 2′-OMe nucleoside may be a stereo-standard nucleoside or a stereo-non-standard nucleoside.

As used herein, “2′-OMe sugar moiety” means the sugar moiety of a 2′-OMe nucleoside.

“2′-F nucleoside” means a nucleoside according to the structure:

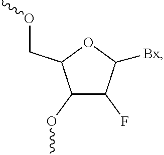

wherein Bx is a nucleobase.

A 2′-F nucleoside may be a stereo-standard nucleoside or a stereo-non-standard nucleoside.

As used herein, “2′-F sugar moiety” means the sugar moiety of a 2′-F nucleoside.

As used herein, “2′-NMA nucleoside” means a nucleoside according to the structure:

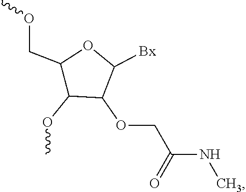

wherein Bx is a nucleobase.

As used herein, “2′-NMA sugar moiety” means the sugar moiety of a 2′-NMA nucleoside as defined herein.

As used herein, “motif” means the pattern of unmodified and/or modified sugar moieties, nucleobases, and/or internucleoside linkages, in an oligonucleotide.

As used herein, “nucleobase” means an unmodified nucleobase or a modified nucleobase. As used herein an “unmodified nucleobase” is unmodified adenine (A), unmodified thymine (T), unmodified cytosine (C), unmodified uracil (U), or unmodified guanine (G). The terms “adenine”. “thymine”, “cytosine”, “uracil”, and “guanine” refer, respectively, to unmodified adenine, unmodified thymine, unmodified cytosine, unmodified uracil, and unmodified guanine, unless otherwise indicated. As used herein, a modified nucleobase is a group of atoms capable of pairing with at least one unmodified nucleobase. A universal base is a nucleobase that can pair with any one of the five unmodified nucleobases. 5-methylcytosine (^{m}C) is one example of a modified nucleobase.

As used herein, “nucleobase sequence” means the order of contiguous nucleobases in a nucleic acid or oligonucleotide independent of any sugar moiety or internucleoside linkage modification. Unless otherwise specified, uracil nucleobases are interchangeable with thymine (and vice versa), and cytosine nucleobases are interchangeable with 5-methylcytosine (and vice versa).

As used herein, “nucleoside” means a moiety comprising a nucleobase and optionally a sugar moiety. The nucleobase and sugar moiety are each, independently, unmodified or modified. As used herein, “modified nucleoside” means a nucleoside comprising a modified nucleobase and/or a modified sugar moiety. A modified nucleoside may comprise a conjugate group.

As used herein, “oligomeric compound” means a compound consisting of (1) an oligonucleotide such as a modified oligonucleotides (a single-stranded oligomeric compound) and (2) optionally one or more additional features, such as a conjugate group or terminal group.As used herein, “oligomeric duplex” means a complex of two oligomeric compounds which are at least partially complementary to each other. In certain embodiments, an oligomeric duplex is an antisense agent, and comprises an antisense oligonucleotide and a sense oligonucleotide.

As used herein, “oligonucleotide” means a strand of linked nucleosides connected via internucleoside linkages, wherein each nucleoside and internucleoside linkage may be modified or unmodified. An oligonucleotide may comprise a self-complementary region. Unless otherwise indicated, oligonucleotides consist of 12-80 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, liquids, powders, or suspensions that can be aerosolized or otherwise dispersed for inhalation by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds (including oligomeric compounds that are antisense agents or portions thereof), i.e., salts that retain the desired biological activity of the compound and do not impart undesired toxicological effects thereto.

As used herein “pharmaceutical composition” means a mixture of substances suitable for administering to a subject. For example, a pharmaceutical composition may comprise an antisense agent and an aqueous solution.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNA (ssRNA), and microRNA, including microRNA mimics. RNAi agents may comprise conjugate groups and/or terminal groups. In certain embodiments, an RNAi agent modulates the amount, activity, and/or splicing of a target nucleic acid. The term RNAi agent excludes antisense agents that act through RNase H.

As used herein, “RNAi oligonucleotide” means an antisense RNAi modified oligonucleotide or a sense RNAi modified oligonucleotide.

As used herein, “antisense RNAi oligonucleotide” means an oligonucleotide comprising a region that is complementary to a target sequence, and which includes at least one chemical modification suitable for RNAi.

As used herein, “antisense RNAi oligomeric compound” means a single-stranded oligomeric compound comprising a region that is complementary to a target sequence, and which includes at least one chemical modification suitable for RNAi.

As used herein, “sense RNAi oligonucleotide” means an oligonucleotide comprising a region that is complementary to a region of an antisense RNAi modified oligonucleotide, and which is capable of forming an oligomeric duplex with such antisense RNAi modified oligonucleotide.

As used herein, “sense RNAi oligomeric compound” means a single-stranded oligomeric compound comprising a region that is complementary to a region of an antisense RNAi modified oligonucleotide and/or an antisense RNAi oligomeric compound, and which is capable of forming an oligomeric duplex with such antisense RNAi modified oligonucleotide and/or antisense RNAi oligomeric compound.

An oligomeric duplex formed by an antisense RNAi oligonucleotide and/or an antisense RNAi oligomeric compound with a sense RNAi oligonucleotide and/or a sense RNAi oligomeric compound is referred to as a double-stranded RNAi agent (dsRNAi) or a short interfering RNA (siRNA) or an RNAi duplex.

As used herein, the term “seed region” in reference to an antisense RNAi oligonucleotide refers to a region at or near the 5′end of an antisense RNAi oligonucleotide having a nucleobase sequence that is important for target nucleic acid recognition by the antisense RNAi oligonucleotide. In certain embodiments, a seed region comprises nucleobases 3-8, nucleobases 2-8, nucleobases 3-7, nucleobases 2-7, nucleobases 1-7, nucleobases 1-6, nucleobases 1-7, or nucleobases 1-8 of an antisense RNAi oligonucleotide, counting from the 5′-terminal nucleoside as position 1.

As used herein, the term “single-stranded” in reference to an oligomeric compound means such a compound consisting of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case the compound would no longer be single-stranded.

As used herein, “stereo-standard nucleoside” means a nucleoside comprising a non-bicyclic furanosyl sugar moiety having the configuration of naturally occurring DNA and RNA nucleosides, according to the formula:

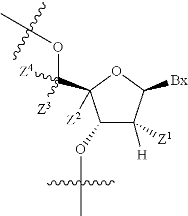

in which Z¹ is H or a 2′-substituent group, and Z², Z³, and Z⁴ are optional substituent groups as described herein and as known in the art. In certain embodiments, Z¹ is a 2′-substituent and Z²-Z⁴ are each H. In certain embodiments, Z¹-Z⁴ are each H (“2′-(3-D-deoxyribosyl nucleoside”). In certain embodiments, Z¹ is a 2′-substituent selected from: OH (“3-D-ribosyl nucleoside” or “stereo-standard deoxy nucleoside”), F (“ribo-2′-F nucleoside” or “stereo-standard 2′-F nucleoside”), OMe (“ribo-2′-OMe nucleoside” or “stereo-standard 2′-OMe nucleoside”), —OCH₂CH₂OCH₃ (“ribo-2′-MOE nucleoside” or “stereo-standard 2′-MOE nucleoside”), and —OCH₂C(═O)—N(H)CH₃ (“ribo-2′-NMA nucleoside” or “stereo-standard 2′-NMA nucleoside”). In certain embodiments, the 2′-substituent is selected from OMe, F, OCH₂CH₂OCH₃, O-alkyl, SMe, or NMA. In certain embodiments, Z¹-Z³ are H and Z⁴ is a 5′-substituent selected from methyl, allyl, or ethyl, optionally methyl. In certain embodiments. Z¹ and Z² join to form a bridge, and the stereo-standard nucleoside is a bicyclic nucleoside. In certain embodiments, the Z¹-Z² bridge is a 2′-O—CH₂-4′ (“LNA”). In certain embodiments, the Z¹-Z² bridge is a 2′-O—CH₂(CH₃)-4′ (“cEt”). In certain embodiments, the heterocyclic base moiety Bx is selected from uracil, thymine, cytosine, 5-methyl cytosine, adenine, and guanine. In certain embodiments, the heterocyclic base moiety Bx is selected from uracil, thymine, cytosine, 5-methyl cytosine, adenine, guanine, and hypoxanthine. In certain embodiments, the heterocyclic base moiety Bx is selected from uracil, thymine, cytosine, adenine, guanine, and hypoxanthine. In certain embodiments, the heterocyclic base moiety Bx is selected from uracil, thymine, cytosine, adenine, and guanine. In certain embodiments, the heterocyclic base moiety Bx is selected from uracil, cytosine, adenine, and guanine. In certain embodiments, the heterocyclic base moiety Bx is a modified nucleobase as described herein or as known in the art.

As used herein, a “stereo-standard sugar moiety” is the sugar moiety of a stereo-standard nucleoside.

As used herein, “stereo-non-standard nucleoside” means a nucleoside comprising a non-bicyclic furanosyl sugar moiety having a configuration other than that of a stereo-standard sugar moiety. In some embodiments, a “stereo-non-standard RNA nucleoside” has Formula X. In certain embodiments, a “stereo-non-standard nucleoside” is represented by one of Formulas I-VII. A “stereo-non-standard sugar moiety” means the sugar moiety of a stereo-non-standard nucleoside.

As used herein, “stabilized phosphate group” refers to a terminal group that results in stabilization of a 5′-phosphate moiety of the 5′-terminal nucleoside of an oligonucleotide, relative to the stability of an unmodified 5′-phosphate of an unmodified nucleoside under biologic conditions. Such stabilization of a 5′-phophate group includes but is not limited to resistance to removal by phosphatases. Stabilized phosphate groups include, but are not limited to, 5′-vinyl phosphonates, 5′-methyl phosphonates, and 5′-cyclopropyl phosphonate.

As used herein, “stereorandom” or “stereorandom chiral center” in the context of a population of molecules of identical molecular formula means a chiral center that is not controlled during synthesis, or enriched following synthesis, for a particular absolute stereochemical configuration. The stereochemical configuration of a chiral center is random when it is the result of a synthetic method that is not designed to control the stereochemical configuration. For example, in a population of molecules comprising a stereorandom chiral center, the number of molecules having the (S) configuration of the stereorandom chiral center may be the same as the number of molecules having the (R) configuration of the stereorandom chiral center (“racemic”). In certain embodiments, the stereorandom chiral center is not racemic because one absolute configuration predominates following synthesis, e.g., due to the action of non-chiral reagents near the enriched stereochemistry of an adjacent sugar moiety. In certain embodiments, the stereorandom chiral center is at the phosphorous atom of one or more of a stereorandom phosphorothioate internucleoside linkage, or a mesyl phosphoramidate internucleoside linkage.

As used herein, “subject” means a human or non-human animal selected for treatment or therapy.

As used herein, “sugar moiety” means an unmodified sugar moiety or a modified sugar moiety. As used herein, “unmodified sugar moiety” means a (3-D-ribosyl moiety, as found in naturally occurring RNA, or a 2′-(3-D-deoxyribosyl sugar moiety as found in naturally occurring DNA. As used herein, “modified sugar moiety” or “modified sugar” means a sugar surrogate or a furanosyl sugar moiety other than a (3-D-ribosyl or a 2′-β-D-deoxyribosyl, which are modified or substituted at a position(s) of the sugar moiety, or may be unsubstituted, and they may be, e.g., stereo-standard or stereo-non-standard sugar moieties. Modified sugar moieties include bicyclic sugars and 2′-substituted sugars.

As used herein, “sugar surrogate” means a modified sugar moiety that does not comprise a tetrahydrofuranyl ring (is not a stereo-standard sugar moiety, nor a stereo-non-standard sugar moiety) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids. Examples of sugar surrogates are provided herein.

As used herein, “target nucleic acid,” “target RNA,” “target RNA transcript” and “nucleic acid target” means a nucleic acid that an oligomeric compound, such as an antisense compound, is designed to affect. In certain embodiments, an oligomeric compound comprises an oligonucleotide having a nucleobase sequence that is complementary to more than one RNA, only one of which is the target RNA of the oligomeric compound. In certain embodiments, the target RNA is an RNA present in the species to which an oligomeric compound is administered.

As used herein, “therapeutic index” means a comparison of the amount of a compound that causes a therapeutic effect to the amount that causes toxicity. Compounds having a high therapeutic index have strong efficacy and low toxicity. In certain embodiments, increasing the therapeutic index of a compound increases the amount of the compound that can be safely administered.

As used herein, “treat” refers to administering a compound or pharmaceutical composition to an animal in order to effect an alteration or improvement of a disease, disorder, or condition in the animal.

As used herein, “alkyl” refers to a saturated straight or branched hydrocarbon substituent group containing up to twenty four carbon atoms. Examples of alkyl groups include without limitation, methyl, ethyl, propyl, butyl, isopropyl, n-hexyl, octyl, decyl, dodecyl and the like. Alkyl groups typically include from 1 to 22 carbon atoms (“C₁-C₂₂ alkyl”), more typically from 1 to 12 carbon atoms (“C₁-C₁₂ alkyl”) with from 1 to 6 carbon atoms (“C₁-C₆ alkyl”) being more preferred. Alkyl groups as used herein may optionally include one or more further substituent groups.

As used herein, “alkenyl,” refers to a straight or branched hydrocarbon chain substituent group containing up to twenty four carbon atoms and having at least one carbon-carbon double bond. Examples of alkenyl groups include without limitation, ethenyl, propenyl, butenyl, 1-methyl-2-buten-1-yl, dienes such as 1,3-butadiene and the like. Alkenyl groups typically include from 2 to 20 carbon atoms, more typically from 2 to 12 carbon atoms with from 2 to 6 carbon atoms being more preferred. Alkenyl groups as used herein may optionally include one or more further substituent groups.

As used herein, “alkynyl”, refers to a straight or branched hydrocarbon substituent group containing up to twenty four carbon atoms and having at least one carbon-carbon triple bond. Examples of alkynyl groups include, without limitation, ethynyl, 1-propynyl, 1-butynyl, and the like. Alkynyl groups typically include from 2 to 20 carbon atoms, more typically from 2 to 12 carbon atoms with from 2 to 6 carbon atoms being more preferred. Alkynyl groups as used herein may optionally include one or more further substituent groups.

As used herein, “alkoxy” refers to an alkyl-O— substituent group, where alkyl is as defined herein. Examples of alkoxy groups include without limitation, methoxy, ethoxy, propoxy, isopropoxy, n-butoxy, see-butoxy, tert-butoxy, n-pentoxy, neopentoxy, n-hexoxy and the like. Alkoxy groups as used herein may optionally include further substituent groups.

As used herein, “aryl” refers to a carbocyclic ring system substituent group having one or more aromatic rings. The aryl may be monocyclic or may include two or more fused rings. Examples of aryl groups include without limitation, phenyl, naphthyl, tetrahydronaphthyl, indanyl, idenyl and the like. Preferred aryl ring systems have from 6 to 10 ring atoms. Aryl groups as used herein may optionally include further substituent groups.

As used herein, “cycloalkyl” refers to a saturated or unsaturated carbocyclic ring system substituent group that does not include an aromatic ring. The cycloalkyl may be monocyclic or may include two or more fused rings. Examples of cycloalkyl groups include without limitation, cyclopropyl, cyclobutyl, cyclopentyl, cyclohexyl, cyclohexenyl, and the like. Preferred cycloalkyl ring systems have from 3 to 10 ring atoms (“C₃-C₁₀ cycloalkyl”). Cycloalkyl groups as used herein may optionally include further substituent groups.

As used herein, “halo” or “halogen” refers to a substituent group selected from fluoride, chloride, bromide and iodide.

As used herein, “heteroaryl” refers to a substituent group comprising a ring system in which at least one of the rings is aromatic, and at least one ring includes one or more ring heteroatoms. The heteroaryl may be monocyclic or may include two or more fused rings. Heteroaryl groups include at least one ring atom selected from sulfur, nitrogen or oxygen, wherein the sulfur is optionally present as a sulfoxide or sulfone, and wherein the nitrogen is optionally present as an N-oxide. Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, thiophenyl, furanyl, quinolinyl, and the like. Heteroaryl groups as used herein may optionally include further substituent groups.

As used herein, “heteroalkyl” refers to an alkyl substituent group as defined herein in which one or more CH₂ units are replaced with a heteroatom independently selected from O, NH, N(C₁₋₆alkyl), S, SO, and SO₂, except that heteroalkyl does not encompass groups defined herein as alkoxy. Examples of heteroalkyl groups include without limitation, methoxypropyl, ethoxymethyl, propylsulfonyl, 1-(methylthio)propan-2-yl, methyl(methylthio)amino, N-propylamino, 2-(methylamino)ethyl, and the like. Heteroalkyl groups typically include from 1 to 20 carbon atoms (“C₁-C₂₀ heteroalkyl”), more typically from 1 to 12 carbon atoms (“C₁-C₁₂ heteroalkyl”) with from 1 to 6 carbon atoms (“C₁-C₆ heteroalkyl”) being more preferred. Heteroalkyl groups as used herein may optionally include one or more further substituent groups.

As used herein, “heterocyclyl” refers to a substituent group comprising a ring system in which none of the rings are aromatic, and at least one ring includes one or more ring heteroatoms. Heterocyclyl is also meant to include fused ring systems including systems where one or more of the fused rings contain no heteroatoms. Heterocyclyl groups include at least one ring atom selected from sulfur, nitrogen or oxygen, wherein the sulfur is optionally present as a sulfoxide or sulfone. Examples ofheterocyclyl groups include without limitation, morpholino, oxirane, tetrahydropyranyl, tetrahydrothienyl, sulfolanyl, and the like. Heterocyclyl groups as used herein may optionally include further substituent groups.

The term “substituted”, with respect to chemical groups means, unless otherwise indicated, a group is substituted with 1, 2, 3, 4, or 5 or more substituent groups selected from halo (e.g., perhalo), hydroxy, azido, SH, CN, OCN, nitro. C₁-C₂₀ alkyl (e.g., C₁-C₂ alkyl), C₁-C₁₀ substituted alkyl (e.g., CF₃), C₂-C₁₀ alkenyl, C₂-C₁₃ alkynyl, C₁-C₁₀ heteroalkyl, C₁-C₁₀ alkoxy, C₁-C₁₀ substituted alkoxy (e.g., OCF₃), S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, aralkyl, O-aralkyl, C₃₋₁₀cycloalkyl, C₆₋₁₀aryl, heterocyclyl, heteroaryl. N(Rₘ)(Rₙ), C(O)N(Rₘ)(Rₙ), N(Rₘ)(Rₙ)C(O)Rₘ, S(O)₂N(Rₘ)(Rₙ). N(Rₘ)(Rₙ)S(O)₂Rₘ, OC(O)N(Rₘ)(Rₙ), N(Rₘ)C(O)N(Rₘ)(Rₙ), N(Rₘ)C(O)ORₙ, C(O)ORₘ, and OC(O)Rₘ, where each Rₘ and Rₙ is, independently, H, OH, an amino protecting group, or substituted or unsubstituted C₁-C₁₀ alkyl. Substituent groups of this paragraph can be unsubstituted or further substituted at a carbon atom with one or more groups independently selected from: hydroxyl, amino, alkoxy, carboxy, benzyl, phenyl, cyano, nitro (NO₂), thiol, thioalkoxy, thioalkyl, halogen, alkyl, aryl, alkenyl and alkynyl.

### Certain Embodiments

The present disclosure provides the following non-limiting embodiments:
Embodiment 1. An RNAi agent comprising an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, wherein the antisense RNAi oligonucleotide comprises within its seed region at least one stereo non-standard nucleoside having Formula X:

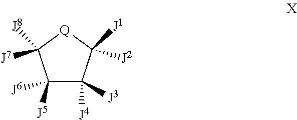

wherein one of J¹ and J² is H and the other is a heterocyclic base moiety Bx:
one of J³ and J⁴ is H and the other is a 2′-substituent selected from H, OH, OMe, F, and OCH₂CH₂OCH₃:
one of J⁵ and J⁶ is H and the other is —O-5′ linkage;
one of J⁷ and J⁸ is H and the other is —C(R¹)₂O-3′ linkage;
   wherein each R¹ is independently H or C₁₋₆ alkyl;
provided that when J² and J⁸ are both H, then one of J³ and J⁵ is not H:
and provided that when Q is O and J² is H, then J⁸ is not H;
Q is O, S, or NR²;
   wherein R² is H, C₁₋₆ alkyl, acetyl, or methanesulfonyl; and
Bx is a heterocyclic base moiety.
Embodiment 2. An RNAi agent for use in a method of comprising administering to a subject in need thereof a composition comprising the RNAi agent and a pharmaceutically acceptable carrier or diluent, optionally in a therapeutically effective amount thereof, wherein the RNAi agent comprises an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, wherein the antisense RNAi oligonucleotide comprises within its seed region at least one stereo non-standard nucleoside having Formula X:

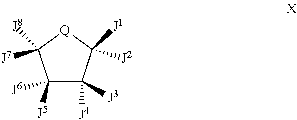

wherein one of J¹ and J² is H and the other is a heterocyclic base moiety Bx:
one of J³ and J⁴ is H and the other is a 2′-substituent selected from H, OH, OMe, F, and OCH₂CH₂OCH₃:
one of J⁵ and J⁶ is H and the other is —O-5′ linkage to the remainder of the oligonucleotide; one of J⁷ and J⁸ is H and the other is —C(R¹)₂O-3′ linkage to the remainder of the oligonucleotide;
   wherein each R¹ is independently H or C₁₋₆ alkyl;
provided that when Q is O and J² and J⁸ are both H, then one of J³ and J⁵ is not H:
Q is O, S, or NR²; and
   wherein R² is H, C₁₋₆ alkyl, or acetyl.
Embodiment 3. The RNAi agent of embodiment 1 or 2, wherein the nucleoside having Formula X is one of nucleoside positions 3-8 of the antisense RNAi oligonucleotide, counting from the 5′-terminus thereof.
Embodiment 4. The RNAi agent of any of the preceding embodiments, wherein Q is O.
Embodiment 5. The RNAi agent of any of the preceding embodiments, wherein each R¹ is H.
Embodiment 6. The RNAi agent of any of the preceding embodiments, wherein each R² is H.
Embodiment 7. The RNAi agent of any of the preceding embodiments, wherein J² and J⁷ are each H.
Embodiment 8. The RNAi agent of any of the preceding embodiments, wherein J⁶ is H.
Embodiment 9. The RNAi agent of any of the preceding embodiments, wherein Bx is selected from uracil, thymine, cytosine, 5-methyl cytosine, adenine, and guanine.
Embodiment 10. The RNAi agent of any of the preceding embodiments, wherein J³ and J⁴ are each H.
Embodiment 11. The RNAi agent of any of embodiments 1 to 9, wherein one of J³ and J⁴ is H and the other is selected from OH. OMe, F, and OCH₃CH₃OCH₃.
Embodiment 12. The RNAi agent of any of embodiments 1 to 9, wherein one of J and J⁴ is H and the other is selected from OMe and F.
Embodiment 13. The RNAi agent of any of embodiments 1 to 9, wherein one of J³ and J⁴ is H and the other is F.
Embodiment 14. The RNAi agent of any of embodiments 1 to 9, wherein one of J³ and J⁴ is H and the other is OMe.
Embodiment 15. The RNAi agent of any of embodiments 1 to 9, wherein one of J³ and J⁴ is H and the other is OH.
Embodiment 16. The RNAi agent of any of the preceding embodiments, wherein the stereo-non-standard nucleoside has a structure selected from Formula I, Formula II, Formula III, Formula IV, Formula V, Formula VI, and Formula VII:

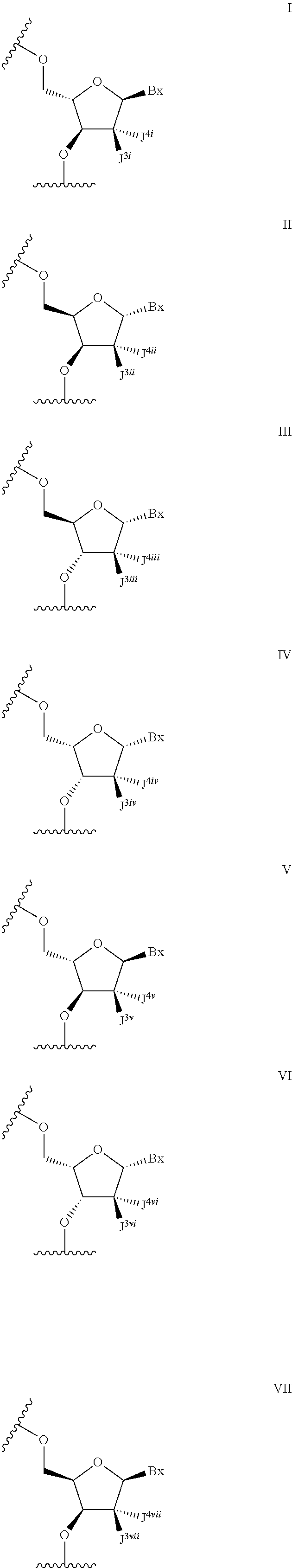

wherein J³ⁱ, J³ⁱⁱ, J³ⁱⁱⁱ, J^{3iv}, J^{3v}, J^{3vi}, and J^{3vii} are as defined for J³ in Formula X; and J⁴ⁱ, J⁴ⁱⁱ, J⁴ⁱⁱⁱ, J^{4iv}, J^{4v}, J^{4vi}, and J^{4vii} are as defined for J⁴ in Formula X; and Bx is a heterocyclic base moiety.
Embodiment 17. The RNAi agent of any of the preceding embodiments, wherein the stereo-non-standard nucleoside has a structure of Formula I.
Embodiment 18. The RNAi agent of embodiment 17, J³ⁱ and J⁴ⁱ are each H.
Embodiment 19. The RNAi agent of any of the preceding embodiments, wherein exactly one nucleoside of the modified oligonucleotide is a stereo-non-standard nucleoside.
Embodiment 20. The RNAi agent of any of the preceding embodiments, wherein 2, 3, 4, 5, or 6 nucleosides of the modified oligonucleotide are stereo-non-standard nucleosides.
Embodiment 21. The RNAi agent of any of the preceding embodiments, wherein nucleoside 6 from the 5′-terminus of the antisense RNAi oligonucleotide is a stereo-non-standard nucleoside, optionally wherein the remaining nucleosides in the antisense RNAi oligonucleotide are stereo-standard nucleosides.
Embodiment 22. The RNAi agent of any of the preceding embodiments, wherein nucleoside 3 from the 5′-terminus of the antisense RNAi oligonucleotide is a stereo-non-standard nucleoside, optionally wherein the remaining nucleosides in the antisense RNAi oligonucleotide are stereo-standard nucleosides.
Embodiment 23. The RNAi agent of any of the preceding embodiments, wherein nucleoside 4 from the 5′-terminus of the antisense RNAi oligonucleotide is a stereo-non-standard nucleoside, optionally wherein the remaining nucleosides in the antisense RNAi oligonucleotide are stereo-standard nucleosides.
Embodiment 24. The RNAi agent of any of the preceding embodiments, wherein nucleoside 5 from the 5′-terminus of the antisense RNAi oligonucleotide is a stereo-non-standard nucleoside, optionally wherein the remaining nucleosides in the antisense RNAi oligonucleotide are stereo-standard nucleosides.
Embodiment 25. The RNAi agent of any of the preceding embodiments, wherein nucleoside 7 from the 5′-terminus of the antisense RNAi oligonucleotide is a stereo-non-standard nucleoside, optionally wherein the remaining nucleosides in the antisense RNAi oligonucleotide are stereo-standard nucleosides.
Embodiment 26. The RNAi agent of any of the preceding embodiments, wherein nucleoside 8 from the 5′-terminus of the antisense RNAi oligonucleotide is a stereo-non-standard nucleoside, optionally wherein the remaining nucleosides in the antisense RNAi oligonucleotide are stereo-standard nucleosides.
Embodiment 27. The RNAi agent of any of the preceding embodiments, wherein each nucleoside in the sense RNAi oligonucleotide is a stereo-standard nucleoside.
Embodiment 28. The RNAi agent of any of the preceding embodiments, wherein the stereo-non-standard nucleoside is linked on at least one of the 3′- or 5′-position with a phosphodiester, phosphorothioate, or mesyl phosphoramidate interucleoside linkage.
Embodiment 29. The RNAi agent of any of the preceding embodiments, wherein the stereo-non-standard nucleoside is linked on at least one of the 3′- or 5′-position with a phosphodiester internucleoside linkage.
Embodiment 30. The RNAi agent of any of the preceding embodiments, wherein the stereo-non-standard nucleoside is linked at both the 3′- or 5′-positions with a phosphodiester internucleoside linkage.
Embodiment 31. The RNAi agent of any one of embodiments 1-29, wherein the stereo-non-standard nucleoside is linked on at least one of the 3′- or 5′-position with a phosphorothioate internucleoside linkage.
Embodiment 32. The RNAi agent of any of the preceding embodiments, wherein each remaining nucleoside in the antisense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside.
Embodiment 33. The RNAi agent of embodiment 32, wherein each bicyclic nucleoside is independently selected from an LNA nucleoside, a cEt nucleoside, and an ENA nucleoside.
Embodiment 34. The RNAi agent of any of the preceding embodiments, wherein each remaining nucleoside in the antisense RNAi oligonucleotide is a stereo-standard nucleoside independently selected from: a 2~-OMe nucleoside, a 2′-F nucleoside, a 2′-MOE nucleoside, and a 2′-deoxynucleoside.
Embodiment 35. The RNAi agent of any of the preceding embodiments, wherein at least one nucleoside in the antisense RNAi oligonucleotide is a 2′-OMe nucleoside.
Embodiment 36. The RNAi agent of any of the preceding embodiments, wherein one, two, or three of the nucleosides at positions 3, 4, 5 from the 5′-terminus of the antisense RNAi oligonucleotide are 2′-OMe nucleosides.
Embodiment 37. The RNAi agent of any of the preceding embodiments, wherein each of the nucleosides at positions 3, 4, 5 from the 5′-terminus of the antisense RNAi oligonucleotide are 2′-OMe nucleosides.
Embodiment 38. The RNAi agent of any of the preceding embodiments, wherein one, two, three, four, or five of the nucleosides at positions 7, 8, 9, 10, and 11 from the 5′-terminus of the antisense RNAi oligonucleotide are 2′-OMe nucleosides.
Embodiment 39. The RNAi agent of any of the preceding embodiments, wherein each of the nucleosides at positions 7, 8, 9, 10, and 11 from the 5′-terminus of the antisense RNAi oligonucleotide are 2′-OMe nucleosides.
Embodiment 40. The RNAi agent of any of the preceding embodiments, wherein one, two, three, four, or five of the nucleosides at positions 17, 18, 19, 20, and 21 from the 5′-terminus of the antisense RNAi oligonucleotide are 2′-OMe nucleosides.
Embodiment 41. The RNAi agent of any of the preceding embodiments, wherein each of the nucleosides at positions 17, 18, 19, 20, and 21 from the 5′-terminus of the antisense RNAi oligonucleotide are 2′-OMe nucleosides.
Embodiment 42. The RNAi agent of any of the preceding embodiments, wherein at least one nucleoside in the antisense RNAi oligonucleotide is a 2′-F nucleoside.
Embodiment 43. The RNAi agent of any of the preceding embodiments, wherein one, two, or three of the nucleosides at positions 2, 14, and 16 from the 5′-terminus of the antisense RNAi oligonucleotide are 2′-F nucleosides.
Embodiment 44. The RNAi agent of any of the preceding embodiments, wherein each of the nucleosides at positions 2, 14, and 16 from the 5′-terminus of the antisense RNAi oligonucleotide are 2′-F nucleosides.
Embodiment 45. The RNAi agent of any of the preceding embodiments, wherein the nucleoside at position 6 from the 5′-terminus of the antisense RNAi oligonucleotide is a 2′-F nucleoside.
Embodiment 46. The RNAi agent of any of the preceding embodiments, wherein the nucleoside at position 6 from the 5′-terminus of the antisense RNAi oligonucleotide is a 2′-OMe nucleoside.
Embodiment 47. The RNAi agent of any of the preceding embodiments, wherein one, two, three, four, or five of the nucleosides at positions 1 from the 5′-terminus of the antisense RNAi oligonucleotide and positions 1, 2, 3, 4, or 5 from the 3′-terminus of the antisense RNAi oligonucleotide are 2′-MOE nucleosides.
Embodiment 48. The RNAi agent of any of the preceding embodiments, wherein the nucleoside at position 1 from the 5′-terminus of the antisense RNAi oligonucleotide is a 2′-MOE nucleoside.
Embodiment 49. The RNAi agent of any of the preceding embodiments, wherein each of the nucleosides at positions 1from the 5′-terminus of the antisense RNAi oligonucleotide and positions 1 and 2 from the 3′-end of the antisense RNAi oligonucleotide are 2′-MOE nucleosides.
Embodiment 50. The RNAi agent of any of the preceding embodiments, wherein one or two of the nucleosides at positions 9 and 10 from the 5′-terminus of the antisense RNAi oligonucleotide are 2′-MOE nucleosides.
Embodiment 51. The RNAi agent of any of the preceding embodiments, wherein the 5′-terminal nucleoside of the antisense RNAi oligonucleotide comprises a stabilized phosphate group.
Embodiment 52. The RNAi agent of embodiment 51, wherein the stabilized phosphate group is 5′-vinyl phosphonate, optionally E-5′-vinyl phosphonate.
Embodiment 53. The RNAi agent of embodiment 51, wherein the stabilized phosphate group is 5′-cyclopropyl phosphonate.
Embodiment 54. The RNAi agent of any of the preceding embodiments, wherein the antisense RNAi oligonucleotide consists of 21-23, optionally 23, linked nucleosides.
Embodiment 55. The RNAi agent of any of the preceding embodiments, wherein each nucleoside in the sense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside.
Embodiment 56. The RNAi agent of embodiment 55, wherein each bicyclic nucleoside is independently selected from an LNA nucleoside, a cEt nucleoside, and an ENA nucleoside.
Embodiment 57. The RNAi agent of any of the preceding embodiments, wherein each nucleoside in the sense RNAi oligonucleotide is a stereo-standard nucleoside independently selected from: a 2′-OMe nucleoside, a 2′-F nucleoside, a 2′-MOE nucleoside, and a 2′-deoxynucleoside.
Embodiment 58. The RNAi agent of any of the preceding embodiments, wherein at least one nucleoside in the sense RNAi oligonucleotide is a 2′-OMe nucleoside.
Embodiment 59. The RNAi agent of any of the preceding embodiments, wherein at least one of the nucleosides at positions 3-6 from the 5′-terminus of the sense RNAi oligonucleotide are 2′-OMe nucleosides, optionally wherein each of the nucleosides at positions 3-8 is a 2′-OMe nucleoside.
Embodiment 60. The RNAi agent of any of the preceding embodiments, wherein at least one of the nucleosides at positions 12-19 from the 5′-terminus of the sense RNAi oligonucleotide are 2′-OMe nucleosides, optionally wherein each of the nucleosides at positions 12-19 is a 2′-OMe nucleoside.
Embodiment 61. The RNAi agent of any of the preceding embodiments, wherein at least one nucleoside in the sense RNAi oligonucleotide is a 2′-F nucleoside.
Embodiment 62. The RNAi agent of any of the preceding embodiments, wherein one, two, three, four, or five of the nucleosides at positions 7-11 from the 5′-terminus of the sense RNAi oligonucleotide are 2′-F nucleosides, optionally wherein each of the nucleosides at positions 7-11 is a 2′-F nucleoside.
Embodiment 63. The RNAi agent of any of the preceding embodiments, wherein at least one nucleoside in the sense RNAi oligonucleotide is a 2′-deoxynucleoside.
Embodiment 64. The RNAi agent of any of the preceding embodiments, wherein one, two, three, four, or five of the nucleosides at positions 7-11 from the 5′-terminus of the sense RNAi oligonucleotide are 2′-deoxynucleosides, optionally wherein one of the nucleosides at positions 7-11 is a 2′-deoxynucleoside.
Embodiment 65. The RNAi agent of any of the preceding embodiments, wherein at least one nucleoside in the sense RNAi oligonucleotide is a 2′-MOE nucleoside.
Embodiment 66. The RNAi agent of any of the preceding embodiments, wherein one, two, three, or four of the nucleosides at positions 1, 2, 20, and 21 from the 5′-terminus of the antisense RNAi oligonucleotide are 2′-MOE nucleosides, optionally wherein each of positions 1, 2, 20, and 21 is a 2′-MOE nucleoside.
Embodiment 67. The RNAi agent of any of the preceding embodiments, wherein one, two, three, or four of the nucleosides at positions 1, 2, 18, 19 from the 5′-terminus of the antisense RNAi oligonucleotide are 2′-MOE nucleosides, optionally wherein each of positions 1, 2, 18, and 19 is a 2′-MOE nucleoside.
Embodiment 68. The RNAi agent of any of the preceding embodiments, wherein the sense RNAi oligonucleotide consists of 19-21, optionally 21, linked nucleosides.
Embodiment 69. The RNAi agent of any of the preceding embodiments, wherein the antisense RNAi oligonucleotide comprises one, two, three, or four phosphorothioate internucleoside linkages joining nucleosides at positions selected from 1 and 2, 2 and 3from the 5′-terminus thereof, and positions 1 and 2, 2 and 3 from the 3′-terminus thereof.
Embodiment 70. The RNAi agent of embodiment 69, wherein the antisense RNAi oligonucleotide comprises four phosphorothioate internucleoside linkages.
Embodiment 71. The RNAi agent of any of the preceding embodiments, wherein the sense RNAi oligonucleotide comprises one, two, three, or four phosphorothioate internucleoside linkages joining nucleosides at positions selected from 1 and 2, 2 and 3from the 5′-terminus thereof, and positions 1 and 2, 2 and 3 from the 3′-terminus thereof.
Embodiment 72. The RNAi agent of embodiment 71, wherein the sense RNAi oligonucleotide comprises exactly two phosphorothioate internucleoside linkages.
Embodiment 73. The RNAi agent of any of the preceding embodiments, wherein the antisense RNAi oligonucleotide comprises an overhang of two nucleosides at the 3′-terminus thereof.
Embodiment 74. The RNAi agent of any of the preceding embodiments, wherein each overhanging nucleoside comprises a 2′-MOE sugar moiety or a 2′-deoxy sugar moiety.
Embodiment 75. The RNAi agent of any of the preceding embodiments, wherein the antisense siRNA oligonucleotide has a nucleobase sequence comprising a targeting region comprising at least 15, optionally 15-21, contiguous nucleobases, wherein the nucleobase sequence of targeting region is at least 85% complementary to an equal length portion of the nucleobase sequence of a target RNA.
Embodiment 76. The RNAi agent of embodiment 75, wherein the nucleobase sequence of the targeting region is at least 90% complementary to the target RNA.
Embodiment 77. The RNAi agent of embodiment 75, wherein the nucleobase sequence of the targeting region is at least 95% complementary to the target RNA.
Embodiment 78. The RNAi agent of embodiment 75, wherein the nucleobase sequence of the targeting region is 100% complementary to the target RNA.
Embodiment 79. The RNAi agent of any of embodiments 75-78, wherein the target RNA encodes a protein.
Embodiment 80. The RNAi agent of any of the preceding embodiments, wherein the sense siRNA oligonucleotide is sufficiently complementary to the antisense siRNA oligonucleotide to form a stable duplex at room temperature.
Embodiment 81. The RNAi agent of any of the preceding embodiments, wherein the antisense RNAi oligonucleotide consists of 21 linked nucleosides.
Embodiment 82. The RNAi agent of any of the preceding embodiments, wherein the antisense RNAi oligonucleotide consists of 23 linked nucleosides.
Embodiment 83. The RNAi agent of any of the preceding embodiments, wherein the sense RNAi oligonucleotide consists of 19 linked nucleosides.
Embodiment 84. The RNAi agent of any of the preceding embodiments, wherein the sense RNAi oligonucleotide consists of 21 linked nucleosides.
Embodiment 85. The RNAi agent of any of the preceding embodiments, wherein the RNAi agent comprises a conjugate group.
Embodiment 86. The RNAi agent of embodiment 85, wherein the conjugate group comprises a cell-targeting moiety.
Embodiment 87. The RNAi agent of embodiment 86, wherein the cell-targeting moiety has affinity for an ASGPR receptor or a Tfr1 receptor.
Embodiment 88. A population of RNAi agents of any of the preceding embodiments, wherein the stereo-non-standard nucleoside is chirally enriched for internucleoside linkages having the Rp or Sp configuration.
Embodiment 89. A population of RNAi agents of any of embodiments 1-87, wherein the stereochemical configuration at each internucleoside linkage is stereorandom.
Embodiment 90. A pharmaceutical composition comprising the RNAi agent or population of any of embodiments 1-89 and a pharmaceutically acceptable carrier or diluent.
Embodiment 91. A method comprising contacting a cell with the RNAi agent or population of any of embodiments 1-89 or pharmaceutical composition of embodiment 90.
Embodiment 92. A method of modulating the amount or activity of a target nucleic acid in a cell, comprising contacting the cell with the RNAi agent of any of embodiments 1-89 or pharmaceutical composition of embodiment 90.
Embodiment 93. The method of embodiment 92, wherein the amount or activity of a target nucleic acid is reduced.
Embodiment 94. Use of the RNAi agent, population, or composition of any of embodiments 1-90 for treatment of a disease or condition.
Embodiment 95. Use of the RNAi agent, population, or composition of any of embodiments 1-90 in therapy.
Embodiment 96. Use of the RNAi agent, population, or composition of any of embodiments 1-90 for use in preparation of a medicament for treatment of a disease or condition.

### Additional Embodiments

Embodiment 1. An RNAi agent comprising an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, wherein at least one nucleoside of the seed region of the antisense RNAi oligonucleotide is a one stereo non-standard nucleoside having Formula X:

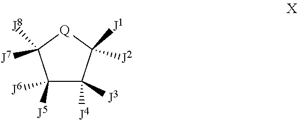

wherein one of J¹ and J² is H and the other is a heterocyclic base moiety Bx:
one of J³ and J⁴ is H and the other is a 2′-substituent selected from H, OH. OMe, F, and OCH₂CH₂OCH₃;
one of J⁵ and J⁶ is H and the other is —O-5′ linkage;
one of J⁷ and J⁸ is H and the other is —C(R¹)₂O-3′ linkage;
   wherein each R¹ is independently H or C₁₋₆ alkyl;
provided that when J² and J⁸ are both H, then one of J³ and J⁵ is not H;
and provided that when Q is O and J² is H, then J is not H;
Q is O, S, or NR²;
   wherein R² is H, C₁₋₆ alkyl, acetyl, or methanesulfonyl; and
Bx is a heterocyclic base moiety.
Embodiment 2. An RNAi agent for use in a method of comprising administering to a subject in need thereof a composition comprising the RNAi agent and a pharmaceutically acceptable carrier or diluent, optionally in a therapeutically effective amount thereof, wherein the RNAi agent comprises an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, wherein at least one nucleoside of the seed region of the antisense RNAi oligonucleotide is a stereo non-standard nucleoside having Formula X:

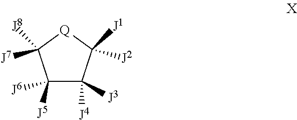

wherein one of J¹ and J² is H and the other is a heterocyclic base moiety Bx;
one of J³ and J⁴ is H and the other is a 2′-substituent selected from H, OH. OMe, F, and OCH₂CH₂OCH₃;
one of J⁵ and J⁶ is H and the other is —O-5′ linkage to the remainder of the oligonucleotide;
one of J⁷ and J⁸ is H and the other is —C(R¹)₂O-3′ linkage to the remainder of the oligonucleotide;
   wherein each R¹ is independently H or C₁₋₆ alkyl;
provided that when Q is O and J² and J⁸ are both H, then one of J³ and J⁵ is not H;
Q is O, S, or NR²; and
   wherein R² is H, C₁₋₆ alkyl, or acetyl.
Embodiment 3. The RNAi agent of embodiment 2, wherein the subject is a human.
Embodiment 4. The RNAi agent of embodiment 1 or 2, wherein the nucleoside having Formula X is one of nucleoside positions 3-8 of the antisense RNAi oligonucleotide, counting from the 5′-terminus thereof.
Embodiment 5. The RNAi agent of any of the preceding embodiments, wherein Q is O.
Embodiment 6. The RNAi agent of any of the preceding embodiments, wherein each R¹ is H.
Embodiment 7. The RNAi agent of any of the preceding embodiments, wherein each R² is H.
Embodiment 8. The RNAi agent of any of the preceding embodiments, wherein J² and J⁷ are each H.
Embodiment 9. The RNAi agent of any of the preceding embodiments, wherein J⁶ is H.
Embodiment 10. The RNAi agent of any of the preceding embodiments, wherein Bx is selected from uracil, thymine, cytosine, 5-methyl cytosine, adenine, guanine, and hypoxanthine.
Embodiment 11. The RNAi agent of any of the preceding embodiments, wherein J³ and J⁴ are each H.
Embodiment 12. The RNAi agent of any of embodiments 1 to 9, wherein one of J³ and J⁴ is H and the other is selected from OH. OMe, F, and OCH₂CH₂OCH₃.
Embodiment 13. The RNAi agent of any of embodiments 1 to 9, wherein one of J³ and J⁴ is H and the other is selected from OMe and F.
Embodiment 14. The RNAi agent of any of embodiments 1 to 9, wherein one of J³ and J⁴ is H and the other is F.
Embodiment 15. The RNAi agent of any of embodiments 1 to 9, wherein one of J³ and J⁴ is H and the other is OMe.
Embodiment 16. The RNAi agent of any of embodiments 1 to 9, wherein one of J³ and J⁴ is H and the other is OH.
Embodiment 17. The RNAi agent of any of the preceding embodiments, wherein the stereo-non-standard nucleoside has a structure selected from Formula I. Formula II. Formula III, Formula IV. Formula V, Formula VI, and Formula VII:

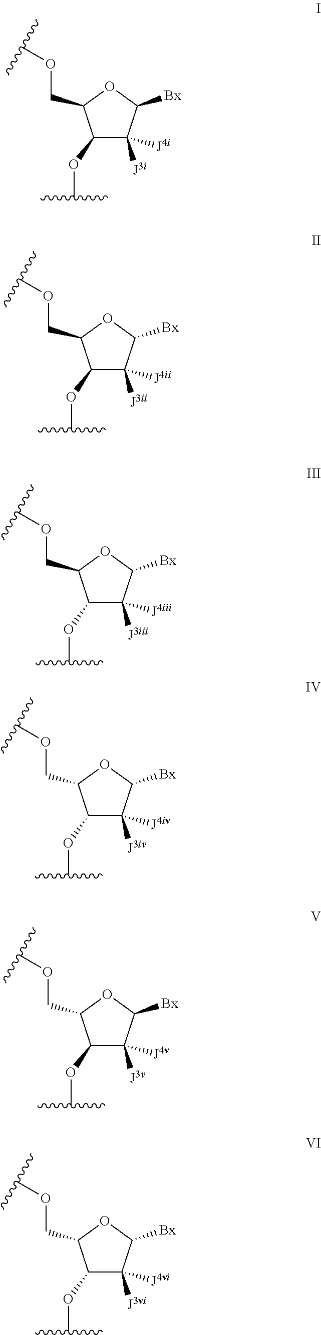

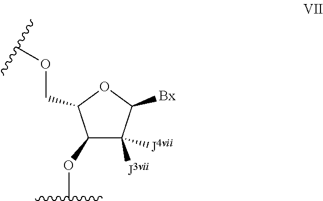

wherein J³ⁱ, J³ⁱⁱ, J³ⁱⁱⁱ, J^{3iv}, J^{3v}, J^{3vi}, and J^{3vii} are as defined for J³ in Formula X; and J⁴ⁱ, J⁴ⁱⁱ, J⁴ⁱⁱⁱ, J^{4iv}, J^{4v}, J^{4vi}, and J^{4v} are as defined for J⁴ in Formula X; and Bx is a heterocyclic base moiety.
Embodiment 18. The RNAi agent of any of the preceding embodiments, wherein the stereo-non-standard nucleoside has a structure of Formula I.
Embodiment 19. The RNAi agent of embodiment 17 or 18, wherein J³ⁱ and J⁴ⁱ are each H.
Embodiment 20. The RNAi agent of embodiment 17 or 18, wherein J³ⁱ is H and J⁴ⁱ is F.
Embodiment 21. The RNAi agent of embodiment 17 or 18, wherein J³ⁱ is H and J⁴ⁱ is OMe.
Embodiment 22. The RNAi agent of any of the preceding embodiments, wherein the stereo-non-standard nucleoside has a structure of Formula VII.
Embodiment 23. The RNAi agent of embodiment 17 or 22, wherein J^{3vii} and J^{4vii} are each H.
Embodiment 24. The RNAi agent of any of the preceding embodiments, wherein exactly one nucleoside of the antisense RNAi oligonucleotide is a stereo-non-standard nucleoside.
Embodiment 25. The RNAi agent of any of the preceding embodiments, wherein 2, 3, 4, 5, or 6 nucleosides of the antisense RNAi oligonucleotide are stereo-non-standard nucleosides.
Embodiment 26. The RNAi agent of any of the preceding embodiments, wherein nucleoside 6 from the 5′-terminus of the antisense RNAi oligonucleotide is a stereo-non-standard nucleoside, optionally wherein the remaining nucleosides in the antisense RNAi oligonucleotide are stereo-standard nucleosides.
Embodiment 27. The RNAi agent of any of the preceding embodiments, wherein nucleoside 3 from the 5′-terminus of the antisense RNAi oligonucleotide is a stereo-non-standard nucleoside, optionally wherein the remaining nucleosides in the antisense RNAi oligonucleotide are stereo-standard nucleosides.
Embodiment 28. The RNAi agent of any of the preceding embodiments, wherein nucleoside 4 from the 5′-terminus of the antisense RNAi oligonucleotide is a stereo-non-standard nucleoside, optionally wherein the remaining nucleosides in the antisense RNAi oligonucleotide are stereo-standard nucleosides.
Embodiment 29. The RNAi agent of any of the preceding embodiments, wherein nucleoside 5 from the 5′-terminus of the antisense RNAi oligonucleotide is a stereo-non-standard nucleoside, optionally wherein the remaining nucleosides in the antisense RNAi oligonucleotide are stereo-standard nucleosides.
Embodiment 30. The RNAi agent of any of the preceding embodiments, wherein nucleoside 7 from the 5′-terminus of the antisense RNAi oligonucleotide is a stereo-non-standard nucleoside, optionally wherein the remaining nucleosides in the antisense RNAi oligonucleotide are stereo-standard nucleosides.
Embodiment 31. The RNAi agent of any of the preceding embodiments, wherein nucleoside 8 from the 5′-terminus of the antisense RNAi oligonucleotide is a stereo-non-standard nucleoside, optionally wherein the remaining nucleosides in the antisense RNAi oligonucleotide are stereo-standard nucleosides.
Embodiment 32. The RNAi agent of any of the preceding embodiments, wherein each nucleoside in the sense RNAi oligonucleotide is a stereo-standard nucleoside.
Embodiment 33. The RNAi agent of any of the preceding embodiments, wherein the stereo-non-standard nucleoside is linked on at least one of the 3′- or 5′-position with a phosphodiester, phosphorothioate, or mesyl phosphoramidate internucleoside linkage.
Embodiment 34. The RNAi agent of any of the preceding embodiments, wherein the stereo-non-standard nucleoside is linked on at least one of the 3′- or 5′-position with a phosphodiester internucleoside linkage.
Embodiment 35. The RNAi agent of any of the preceding embodiments, wherein the stereo-non-standard nucleoside is linked at both the 3′- or 5′-positions with a phosphodiester internucleoside linkage.
Embodiment 36. The RNAi agent of any one of embodiments 1-34, wherein the stereo-non-standard nucleoside is linked on at least one of the 3′- or 5′-position with a phosphorothioate internucleoside linkage.
Embodiment 37. The RNAi agent of any of the preceding embodiments, wherein each remaining nucleoside in the antisense RNAi oligonucleotide is independently selected from: a bicyclic nucleoside, a 2′-OMe nucleoside, a 2-F nucleoside, a 2′-MOE nucleoside, a 2′-NMA nucleoside, a 2′-deoxynucleoside, and a ribosyl nucleoside.
Embodiment 38. The RNAi agent of embodiment 32, wherein each bicyclic nucleoside is independently selected from an LNA nucleoside, a cEt nucleoside, and an ENA nucleoside.
Embodiment 39. The RNAi agent of any of the preceding embodiments, wherein each remaining nucleoside in the antisense RNAi oligonucleotide is a stereo-standard nucleoside independently selected from: a 2′-OMe nucleoside, a 2′-F nucleoside, a 2′-MOE nucleoside, and a 2′-deoxynucleoside.
Embodiment 40. The RNAi agent of any of the preceding embodiments, wherein at least one nucleoside in the antisense RNAi oligonucleotide is a 2′-OMe nucleoside.
Embodiment 41. The RNAi agent of embodiment 40, wherein 8-21 nucleosides in the antisense RNAi oligonucleotide are 2′-OMe nucleosides, optionally wherein 15-21 nucleosides in the antisense RNAi oligonucleotide are 2′-OMe nucleosides.
Embodiment 42. The RNAi agent of any of the preceding embodiments, wherein one, two, or three of the nucleosides at positions 3, 4, and 5 from the 5′-terminus of the antisense RNAi oligonucleotide are 2′-OMe nucleosides.
Embodiment 43. The RNAi agent of any of the preceding embodiments, wherein each of the nucleosides at positions 3, 4, 5 from the 5′-terminus of the antisense RNAi oligonucleotide are 2′-OMe nucleosides.
Embodiment 44. The RNAi agent of any of the preceding embodiments, wherein one, two, three, four, or five of the nucleosides at positions 7, 8, 9, 10, and 11 from the 5′-terminus of the antisense RNAi oligonucleotide are 2′-OMe nucleosides.
Embodiment 45. The RNAi agent of any of the preceding embodiments, wherein each of the nucleosides at positions 7, 8, 9, 10, and 11 from the 5′-terminus of the antisense RNAi oligonucleotide are 2′-OMe nucleosides.
Embodiment 46. The RNAi agent of any of the preceding embodiments, wherein one, two, three, four, or five of the nucleosides at positions 17, 18, 19, 20, and 21 from the 5′-terminus of the antisense RNAi oligonucleotide are 2′-OMe nucleosides.
Embodiment 47. The RNAi agent of any of the preceding embodiments, wherein each of the nucleosides at positions 17, 18, 19, 20, and 21 from the 5′-terminus of the antisense RNAi oligonucleotide are 2′-OMe nucleosides.
Embodiment 48. The RNAi agent of any of the preceding embodiments, wherein at least one nucleoside in the antisense RNAi oligonucleotide is a 2′-F nucleoside.
Embodiment 49. The RNAi agent of embodiment 48, wherein 1-10 nucleosides in the antisense RNAi oligonucleotide are 2′-F nucleosides, optionally wherein 1-6 nucleosides in the antisense RNAi oligonucleotide are 2′-F nucleosides, optionally wherein exactly 1, 2, 3, 4, 5, or 6 nucleosides in the antisense RNAi oligonucleotide are 2′-F nucleosides.
Embodiment 50. The RNAi agent of any of the preceding embodiments, wherein one, two, or three of the nucleosides at positions 2, 14, and 16 from the 5′-terminus of the antisense RNAi oligonucleotide are 2′-F nucleosides.
Embodiment 51. The RNAi agent of any of the preceding embodiments, wherein each of the nucleosides at positions 2, 14, and 16 from the 5′-terminus of the antisense RNAi oligonucleotide are 2′-F nucleosides.
Embodiment 52. The RNAi agent of any of the preceding embodiments, wherein the nucleoside at position 6 from the 5′-terminus of the antisense RNAi oligonucleotide is a 2′-F nucleoside.
Embodiment 53. The RNAi agent of any of the preceding embodiments, wherein the nucleoside at position 6 from the 5′-terminus of the antisense RNAi oligonucleotide is a 2′-OMe nucleoside.
Embodiment 54. The RNAi agent of any of the preceding embodiments, wherein at least one nucleoside in the antisense RNAi oligonucleotide is a 2′-MOE nucleoside.
Embodiment 55. The RNAi agent of embodiment 54, wherein 1-6 nucleosides in the antisense RNAi oligonucleotide are 2′-MOE nucleosides, optionally wherein 1-3 nucleosides in the antisense RNAi oligonucleotide are 2′-MOE nucleosides.
Embodiment 56. The RNAi agent of any of the preceding embodiments, wherein one, two, three, four, or five of the nucleosides at positions 1 from the 5′-terminus of the antisense RNAi oligonucleotide and positions 1, 2, 3, 4, or 5 from the 3′-terminus of the antisense RNAi oligonucleotide are 2′-MOE nucleosides: optionally wherein the antisense RNAi oligonucleotide consists of 23 linked nucleosides.
Embodiment 57. The RNAi agent of any of the preceding embodiments, wherein the nucleoside at position 1 from the 5′-terminus of the antisense RNAi oligonucleotide is a 2′-MOE nucleoside.
Embodiment 58. The RNAi agent of any of the preceding embodiments, wherein each of the nucleosides at positions 1 from the 5′-terminus of the antisense RNAi oligonucleotide and positions 1 and 2 from the 3′-end of the antisense RNAi oligonucleotide are 2′-MOE nucleosides.
Embodiment 59. The RNAi agent of any of the preceding embodiments, wherein one or two of the nucleosides at positions 9 and 10 from the 5′-terminus of the antisense RNAi oligonucleotide are 2′-MOE nucleosides.
Embodiment 60. The RNAi agent of any of the preceding embodiments, wherein the 5′-terminal nucleoside of the antisense RNAi oligonucleotide comprises a stabilized phosphate group.
Embodiment 61. The RNAi agent of embodiment 51, wherein the stabilized phosphate group is 5′-vinyl phosphonate, optionally E-5′-vinyl phosphonate.
Embodiment 62. The RNAi agent of embodiment 51, wherein the stabilized phosphate group is 5′-cyclopropyl phosphonate.
Embodiment 63. The RNAi agent of any of the preceding embodiments, wherein the antisense RNAi oligonucleotide consists of 21-23, optionally 23, linked nucleosides.
Embodiment 64. The RNAi agent of any of the preceding embodiments, wherein each nucleoside in the sense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside.
Embodiment 65. The RNAi agent of embodiment 55, wherein each bicyclic nucleoside is independently selected from an LNA nucleoside, a cEt nucleoside, and an ENA nucleoside.
Embodiment 66. The RNAi agent of any of the preceding embodiments, wherein each nucleoside in the sense RNAi oligonucleotide is a stereo-standard nucleoside independently selected from: a 2′-OMe nucleoside, a 2′-F nucleoside, a 2′-MOE nucleoside, and a 2′-deoxynucleoside.
Embodiment 67. The RNAi agent of any of the preceding embodiments, wherein at least one nucleoside in the sense RNAi oligonucleotide is a 2′-OMe nucleoside.
Embodiment 68. The RNAi agent of embodiment 67, wherein 8-21 nucleosides in the sense RNAi oligonucleotide are 2′-OMe nucleosides, optionally wherein 13-21 nucleosides in the sense RNAi oligonucleotide are 2′-OMe nucleosides.
Embodiment 69. The RNAi agent of any of the preceding embodiments, wherein at least one of the nucleosides at positions 3-6 from the 5′-terminus of the sense RNAi oligonucleotide are 2′-OMe nucleosides, optionally wherein each of the nucleosides at positions 3-8 is a 2′-OMe nucleoside.
Embodiment 70. The RNAi agent of any of the preceding embodiments, wherein at least one of the nucleosides at positions 12-19 from the 5′-terminus of the sense RNAi oligonucleotide are 2′-OMe nucleosides, optionally wherein each of the nucleosides at positions 12-19 is a 2′-OMe nucleoside.
Embodiment 71. The RNAi agent of any of the preceding embodiments, wherein at least one nucleoside in the sense RNAi oligonucleotide is a 2-F nucleoside.
Embodiment 72. The RNAi agent of embodiment 71, wherein 1-11 nucleosides in the sense RNAi oligonucleotide are 2′-F nucleosides, optionally wherein 2-4 nucleosides in the sense RNAi oligonucleotide are 2′-F nucleosides, optionally wherein exactly 2, 3, or 4 nucleosides in the sense RNAi oligonucleotide are 2′-F nucleosides.
Embodiment 73. The RNAi agent of any of the preceding embodiments, wherein one, two, three, or four of the nucleosides at positions 7 and 9-11 from the 5′-terminus of the sense RNAi oligonucleotide are 2′-F nucleosides, optionally wherein each of the nucleosides at positions 7 and 9-11 is a 2′-F nucleoside.
Embodiment 74. The RNAi agent of any of the preceding embodiments, wherein at least one nucleoside in the sense RNAi oligonucleotide is a 2′-deoxynucleoside.
Embodiment 75. The RNAi agent of any of the preceding embodiments, wherein one, two, three, four, or five of the nucleosides at positions 7-11 from the 5′-terminus of the sense RNAi oligonucleotide are 2′-deoxynucleosides, optionally wherein one of the nucleosides at positions 7-11 is a 2′-deoxynucleoside.
Embodiment 76. The RNAi agent of any of the preceding embodiments, wherein at least one nucleoside in the sense RNAi oligonucleotide is a 2′-MOE nucleoside.
Embodiment 77. The RNAi agent of embodiment 71, wherein 1-6 nucleosides in the sense RNAi oligonucleotide are 2′-MOE nucleosides, optionally wherein 2-4 nucleosides in the sense RNAi oligonucleotide are 2′-MOE nucleosides.
Embodiment 78. The RNAi agent of any of the preceding embodiments, wherein one, two, three, or four of the nucleosides at positions 1, 2, 20, and 21 from the 5′-terminus of the sense RNAi oligonucleotide are 2′-MOE nucleosides, optionally wherein each of positions 1, 2, 20, and 21 is a 2′-MOE nucleoside: optionally wherein the sense RNAi oligonucleotide consists of 21 linked nucleosides.
Embodiment 79. The RNAi agent of any of the preceding embodiments, wherein one, two, three, or four of the nucleosides at positions 1, 2, 18, 19 from the 5′-terminus of the sense RNAi oligonucleotide are 2′-MOE nucleosides, optionally wherein each of positions 1, 2, 18, and 19 is a 2′-MOE nucleoside; optionally wherein the sense RNAi oligonucleotide consists of 19 linked nucleosides.
Embodiment 80. The RNAi agent of any of the preceding embodiments, wherein at least one nucleoside in the sense RNAi oligonucleotide is substituted at its 2′-position with a C₁₆-C₂₂ alkyl group, optionally wherein the C₁₆-C₂₂ alkyl group is unsubstituted, optionally wherein the C₁₆-C₂₂ alkyl group is saturated.
Embodiment 81. The RNAi agent of any of the preceding embodiments, wherein the sense RNAi oligonucleotide consists of 14-21 linked nucleosides.
Embodiment 82. The RNAi agent of any of the preceding embodiments, wherein the sense RNAi oligonucleotide consists of 19-21, optionally 21, linked nucleosides.
Embodiment 83. The RNAi agent of any of the preceding embodiments, wherein the antisense RNAi oligonucleotide comprises one, two, three, or four phosphorothioate internucleoside linkages joining nucleosides at positions selected from 1 and 2, 2 and 3 from the 5′-terminus thereof, and positions 1 and 2, 2 and 3 from the 3′-terminus thereof.
Embodiment 84. The RNAi agent of embodiment 83, wherein the antisense RNAi oligonucleotide comprises four phosphorothioate internucleoside linkages.
Embodiment 85. The RNAi agent of any of the preceding embodiments, wherein the sense RNAi oligonucleotide comprises one, two, three, or four phosphorothioate internucleoside linkages joining nucleosides at positions selected from 1 and 2, 2 and 3 from the 5′-terminus thereof, and positions 1 and 2, 2 and 3 from the 3′-terminus thereof.
Embodiment 86. The RNAi agent of embodiment 85, wherein the sense RNAi oligonucleotide comprises exactly two phosphorothioate internucleoside linkages.
Embodiment 87. The RNAi agent of any of the preceding embodiments, wherein the antisense RNAi oligonucleotide comprises an overhang of two nucleosides at the 3′-terminus thereof.
Embodiment 88. The RNAi agent of any of the preceding embodiments, wherein each overhanging nucleoside comprises a 2′-MOE sugar moiety or a 2′-deoxy sugar moiety.
Embodiment 89. The RNAi agent of any of the preceding embodiments, wherein the antisense siRNA oligonucleotide has a nucleobase sequence comprising a targeting region comprising at least 15, optionally 15-21, contiguous nucleobases, wherein the nucleobase sequence of targeting region is at least 85% complementary to an equal length portion of the nucleobase sequence of a target RNA.
Embodiment 90. The RNAi agent of embodiment 89, wherein the nucleobase sequence of the targeting region is at least 90% complementary to the target RNA.
Embodiment 91. The RNAi agent of embodiment 89, wherein the nucleobase sequence of the targeting region is at least 95% complementary to the target RNA.
Embodiment 92. The RNAi agent of embodiment 89, wherein the nucleobase sequence of the targeting region is 100% complementary to the target RNA.
Embodiment 93. The RNAi agent of any of embodiments 89-92, wherein the target RNA encodes a protein.
Embodiment 94. The RNAi agent of any of the preceding embodiments, wherein the sense siRNA oligonucleotide is sufficiently complementary to the antisense siRNA oligonucleotide to form a stable duplex at room temperature in water.
Embodiment 95. The RNAi agent of any of the preceding embodiments, wherein the antisense RNAi oligonucleotide consists of 21 linked nucleosides.
Embodiment 96. The RNAi agent of any of the preceding embodiments, wherein the antisense RNAi oligonucleotide consists of 23 linked nucleosides.
Embodiment 97. The RNAi agent of any of the preceding embodiments, wherein the sense RNAi oligonucleotide consists of 19 linked nucleosides.
Embodiment 98. The RNAi agent of any of the preceding embodiments, wherein the sense RNAi oligonucleotide consists of 21 linked nucleosides.
Embodiment 99. The RNAi agent of any of the preceding embodiments, wherein the RNAi agent comprises a conjugate group.
Embodiment 100. The RNAi agent of embodiment 99, wherein the conjugate group comprises a cell-targeting moiety.
Embodiment 101. The RNAi agent of embodiment 100, wherein the cell-targeting moiety has affinity for an ASGPR receptor or a Tfr1 receptor.
Embodiment 102. An RNAi agent comprising an antisense RNAi oligomeric compound comprising an antisense RNAi oligonucleotide consisting of 20-50 linked nucleosides and optionally a sense RNAi oligomeric compound comprising a sense RNAi oligonucleotide consisting of 15-23 linked nucleosides, wherein the antisense RNAi oligonucleotide comprises at one or more of nucleosides 3-8 from the 5′-terminus thereof a nucleoside having Formula I:

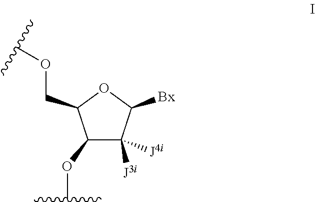

wherein J³ⁱ is H and J⁴ⁱ is selected from H, OH, OMe, F, and OCH₂CH₂OCH₃;
and Bx is a heterocyclic base moiety;
provided that the RNAi agent does not comprise any of the following:

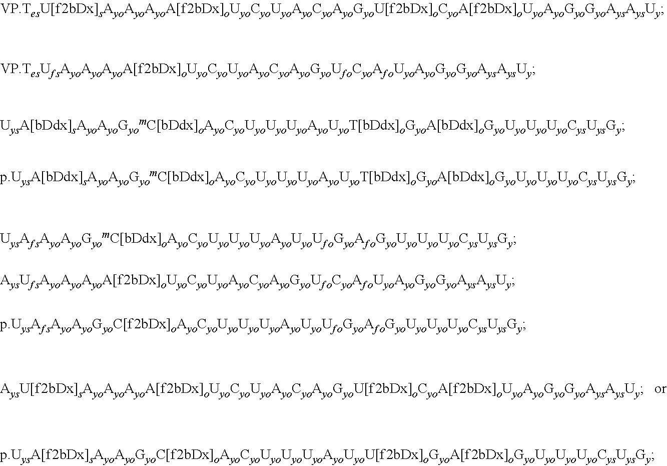

wherein “VP” represents a 5′-vinyl phosphonate moiety, “p” represents a 5′-phosphate moiety, a subscript “[f2bDx]” represents a 2′-fluoro-δ-D-xylosyl sugar moiety, a subscript “f” represents a 2′-fluoro-β-D-ribosyl sugar moiety, a subscript “y” represents a 2′-O-methyl-β-D-ribosyl sugar moiety, a subscript “e” represents a 2′-MOE-O-D-ribosyl sugar moiety, a superscript “m” represents a 5-methyl group on cytosine, a subscript “s” represents a phosphorothioate internucleoside linkage, and a subscript “o” represents a phosphodiester internucleoside linkage.
Embodiment 103. The RNAi agent of embodiment 102, wherein J⁴ⁱ is H.
Embodiment 104. The RNAi agent of embodiment 102, wherein J⁴ⁱ is F.
Embodiment 105. The RNAi agent of embodiment 102, wherein J⁴ⁱ is OMe.
Embodiment 106. The RNAi agent of any embodiments 102-105, wherein exactly one nucleoside of the antisense RNAi oligonucleotide is of Formula I.
Embodiment 107. The RNAi agent of any of embodiments 102-105, wherein 2, 3, 4, 5, or 6 nucleosides of the antisense RNAi oligonucleotide are of Formula I.
Embodiment 108. The RNAi agent of any of embodiments 102-107, wherein nucleoside 6 from the 5′-terminus of the antisense RNAi oligonucleotide is a nucleoside of Formula I, optionally wherein the remaining nucleosides in the antisense RNAi oligonucleotide are stereo-standard nucleosides.
Embodiment 109. The RNAi agent of any of embodiments 102-108, wherein nucleoside 3 from the 5′-terminus of the antisense RNAi oligonucleotide is a nucleoside of Formula I, optionally wherein the remaining nucleosides in the antisense RNAi oligonucleotide are stereo-standard nucleosides.
Embodiment 110. The RNAi agent of any of embodiments 102-109, wherein nucleoside 4 from the 5′-terminus of the antisense RNAi oligonucleotide is a nucleoside of Formula I, optionally wherein the remaining nucleosides in the antisense RNAi oligonucleotide are stereo-standard nucleosides.
Embodiment 111. The RNAi agent of any of embodiments 102-110, wherein nucleoside 5 from the 5′-terminus of the antisense RNAi oligonucleotide is a nucleoside of Formula 1, optionally wherein the remaining nucleosides in the antisense RNAi oligonucleotide are stereo-standard nucleosides.
Embodiment 112. The RNAi agent of any of embodiments 102-111, wherein nucleoside 7 from the 5′-terminus of the antisense RNAi oligonucleotide is a nucleoside of Formula I, optionally wherein the remaining nucleosides in the antisense RNAi oligonucleotide are stereo-standard nucleosides.
Embodiment 113. The RNAi agent of any of embodiments 102-112, wherein nucleoside 8 from the 5′-terminus of the antisense RNAi oligonucleotide is a nucleoside of Formula I, optionally wherein the remaining nucleosides in the antisense RNAi oligonucleotide are stereo-standard nucleosides.
Embodiment 114. The RNAi agent of any of embodiments 102-113, wherein each nucleoside in the sense RNAi oligonucleotide is a stereo-standard nucleoside.
Embodiment 115. The RNAi agent of any of embodiments 102-114, wherein the nucleoside of Formula I is linked on at least one of the 3′- or 5′-position with a phosphodiester, phosphorothioate, or mesyl phosphoramidate internucleoside linkage.
Embodiment 116. The RNAi agent of any of embodiments 102-115, wherein the nucleoside of Formula I is linked on at least one of the 3′- or 5′-position with a phosphodiester internucleoside linkage.
Embodiment 117. The RNAi agent of any of embodiments 102-116, wherein the nucleoside of Formula I is linked at both the 3′- and 5′-positions with a phosphodiester internucleoside linkage.
Embodiment 118. The RNAi agent of any of embodiments 102-116, wherein the nucleoside of Formula I is linked on at least one of the 3′- or 5′-position with a phosphorothioate internucleoside linkage.
Embodiment 119. The RNAi agent of any of embodiment 102-118, wherein each remaining nucleoside in the antisense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside.
Embodiment 120. The RNAi agent of any of embodiments 102-119, wherein each remaining nucleoside in the antisense RNAi oligonucleotide is a stereo-standard nucleoside independently selected from: a 2′-OMe nucleoside, a 2′-F nucleoside, a 2′-MOE nucleoside, and a 2′-deoxynucleoside.
Embodiment 121. The RNAi agent of any of embodiments 102-120, wherein the RNAi agent does not comprise a stereo-non-standard nucleoside outside of positions 3-8 from the 5′-terminus of the antisense RNAi oligonucleotide.
Embodiment 122. The RNAi agent of any of embodiments 102-121, wherein J⁴ is F and the nucleoside of Formula I is at position 5 or 7 from the 5′-terminus of the antisense RNAi oligonucleotide.
Embodiment 123. The RNAi agent of any of embodiments 102-121 for use in treatment of a disease or condition, wherein J⁴ⁱ is F and the nucleoside of Formula I is at position 6 from the 5′-terminus of the antisense RNAi oligonucleotide.
Embodiment 124. The RNAi agent of any of embodiment 123, wherein the treatment comprises administering to a subject in need thereof a composition comprising the RNAi agent and a pharmaceutically acceptable carrier or diluent, optionally in a therapeutically effective amount thereof, optionally wherein the subject is a human.
Embodiment 125. The RNAi agent of any of embodiments 102-121, wherein J⁴ⁱ is H and the nucleoside of Formula I is at position 5 or 7 from the 5′-terminus of the antisense RNAi oligonucleotide.
Embodiment 126. The RNAi agent of any of embodiments 102-121 for use in treatment of a disease or condition, wherein J⁴ⁱ is H and the nucleoside of Formula I is at position 6 from the 5′-terminus of the antisense RNAi oligonucleotide.
Embodiment 127. The RNAi agent of any of embodiment 126, wherein the treatment comprises administering to a subject in need thereof a composition comprising the RNAi agent and a pharmaceutically acceptable carrier or diluent, optionally in a therapeutically effective amount thereof, optionally wherein the subject is a human.
Embodiment 128. The RNAi agent of any of embodiments 102-121, wherein J⁴ⁱ is OMe and the nucleoside of Formula I is at position 5, 6, or 7 from the 5′-terminus of the antisense RNAi oligonucleotide.
Embodiment 129. The RNAi agent of any of embodiment 128, wherein the compound is for use in treatment of a disease or condition, optionally wherein the treatment comprises administering to a subject in need thereof a composition comprising the RNAi agent and a pharmaceutically acceptable carrier or diluent, optionally in a therapeutically effective amount thereof, optionally wherein the subject is a human.
Embodiment 130. The RNAi agent of any of embodiments 102-129, wherein the 5′-terminal nucleoside of the antisense RNAi oligonucleotide comprises a stabilized phosphate group.
Embodiment 131. The RNAi agent of embodiment 130, wherein the stabilized phosphate group is 5′-vinyl phosphonate, optionally E-5′-vinyl phosphonate.
Embodiment 132. The RNAi agent of embodiment 130, wherein the stabilized phosphate group is 5′-cyclopropyl phosphonate.
Embodiment 133. The RNAi agent of any of embodiments 102-132, wherein the antisense RNAi oligonucleotide consists of 21-23, optionally 23, linked nucleosides.
Embodiment 134. The RNAi agent of any of embodiments 102-133, wherein the sense RNAi oligonucleotide consists of 14-21 linked nucleosides.
Embodiment 135. The RNAi agent of any of embodiments 102-134, wherein the sense RNAi oligonucleotide consists of 19-21, optionally 21, linked nucleosides.
Embodiment 136. The RNAi agent of any of embodiments 102-135, wherein Bx is selected from uracil, thymine, cytosine, 5-methyl cytosine, adenine, guanine, and hypoxanthine.
Embodiment 137. The RNAi agent of any of embodiments 102-135, wherein Bx is selected from uracil, thymine, cytosine, adenine, guanine, and hypoxanthine.
Embodiment 138. The RNAi agent of any of embodiments 102-135, wherein Bx is selected from uracil, thymine, cytosine, adenine, and guanine.
Embodiment 139. The RNAi agent of any of embodiments 102-138, wherein each stereo-standard nucleoside in the antisense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside.
Embodiment 140. The RNAi agent of embodiment 139, wherein each bicyclic nucleoside is independently selected from an LNA nucleoside, a cEt nucleoside, and an ENA nucleoside.
Embodiment 141. The RNAi agent of any of embodiments 102-140, wherein each remaining nucleoside in the antisense RNAi oligonucleotide is a stereo-standard nucleoside independently selected from: a 2′-OMe nucleoside, a 2′-F nucleoside, a 2′-MOE nucleoside, and a 2′-deoxynucleoside.
Embodiment 142. The RNAi agent of any of embodiments 102-141, wherein at least one nucleoside in the antisense RNAi oligonucleotide is a 2′-OMe nucleoside.
Embodiment 143. The RNAi agent of any of embodiments 102-142, wherein 8-21 nucleosides in the antisense RNAi oligonucleotide are 2′-OMe nucleosides, optionally wherein 15-21 nucleosides in the antisense RNAi oligonucleotide are 2′-OMe nucleosides.
Embodiment 144. The RNAi agent of any of embodiments 102-143, wherein the nucleoside at position 1 from the 5′-terminus of the antisense RNAi oligonucleotide is a 2′-OMe nucleoside.
Embodiment 145. The RNAi agent of any of embodiments 102-144, wherein one, two, or three of the nucleosides at positions 3, 4, and 5 from the 5′-terminus of the antisense RNAi oligonucleotide are 2′-OMe nucleosides.
Embodiment 146. The RNAi agent of any of embodiments 102-145, wherein each of the nucleosides at positions 3, 4, 5 from the 5′-terminus of the antisense RNAi oligonucleotide are 2′-OMe nucleosides.
Embodiment 147. The RNAi agent of any of embodiments 102-146, wherein one, two, three, four, or five of the nucleosides at positions 7, 8, 9, 10, and 11 from the 5′-terminus of the antisense RNAi oligonucleotide are 2′-OMe nucleosides.
Embodiment 148. The RNAi agent of any of embodiments 102-147, wherein each of the nucleosides at positions 7, 8, 9, 10, and 11 from the 5′-terminus of the antisense RNAi oligonucleotide are 2′-OMe nucleosides.
Embodiment 149. The RNAi agent of any of embodiments 102-148, wherein one, two, three, four, or five of the nucleosides at positions 17, 18, 19, 20, and 21 from the 5′-terminus of the antisense RNAi oligonucleotide are 2′-OMe nucleosides.
Embodiment 150. The RNAi agent of any of embodiments 102-149, wherein each of the nucleosides at positions 17, 18, 19, 20, and 21 from the 5′-terminus of the antisense RNAi oligonucleotide are 2′-OMe nucleosides.
Embodiment 151. The RNAi agent of any of embodiments 102-150, wherein at least one nucleoside in the antisense RNAi oligonucleotide is a 2′-F nucleoside.
Embodiment 152. The RNAi agent of embodiment 151, wherein 1-10 nucleosides in the antisense RNAi oligonucleotide are 2′-F nucleosides, optionally wherein 1-6 nucleosides in the antisense RNAi oligonucleotide are 2′-F nucleosides, optionally wherein exactly 1, 2, 3, 4, 5, or 6 nucleosides in the antisense RNAi oligonucleotide are 2′-F nucleosides.
Embodiment 153. The RNAi agent of any of embodiments 102-152, wherein one, two, or three of the nucleosides at positions 2, 14, and 16 from the 5′-terminus of the antisense RNAi oligonucleotide are 2′-F nucleosides.
Embodiment 154. The RNAi agent of any of embodiments 102-153, wherein each of the nucleosides at positions 2, 14, and 16 from the 5′-terminus of the antisense RNAi oligonucleotide are 2′-F nucleosides.
Embodiment 155. The RNAi agent of any of embodiments 102-154, wherein the nucleoside at position 6 from the 5′-terminus of the antisense RNAi oligonucleotide is a 2′-F nucleoside.
Embodiment 156. The RNAi agent of any of embodiments 102-154, wherein the nucleoside at position 6 from the 5′-terminus of the antisense RNAi oligonucleotide is a 2′-OMe nucleoside.
Embodiment 157. The RNAi agent of any of embodiments 102-156, wherein at least one nucleoside in the antisense RNAi oligonucleotide is a 2′-MOE nucleoside.
Embodiment 158. The RNAi agent of embodiment 157, wherein 1-6 nucleosides in the antisense RNAi oligonucleotide are 2′-MOE nucleosides, optionally wherein 1-3 nucleosides in the antisense RNAi oligonucleotide are 2′-MOE nucleosides.
Embodiment 159. The RNAi agent of any of embodiments 102-158, wherein one, two, three, four, or five of the nucleosides at positions 1 from the 5′-terminus of the antisense RNAi oligonucleotide and positions 1, 2, 3, 4, or 5 from the 3′-terminus of the antisense RNAi oligonucleotide are 2′-MOE nucleosides.
Embodiment 160. The RNAi agent of any of embodiments 102-159, wherein the nucleoside at position 1 from the 5′-terminus of the antisense RNAi oligonucleotide is a 2′-MOE nucleoside.
Embodiment 161. The RNAi agent of any of embodiments 102-160, wherein each of the nucleosides at positions 1 from the 5′-terminus of the antisense RNAi oligonucleotide and positions 1 and 2 from the 3′-end of the antisense RNAi oligonucleotide are 2′-MOE nucleosides.
Embodiment 162. The RNAi agent of any of embodiments 102-161, wherein one or two of the nucleosides at positions 9 and 10 from the 5′-terminus of the antisense RNAi oligonucleotide are 2′-MOE nucleosides.
Embodiment 163. The RNAi agent of any of embodiments 102-162, wherein the 5′-terminal nucleoside of the antisense RNAi oligonucleotide comprises a stabilized phosphate group.
Embodiment 164. The RNAi agent of embodiment 163, wherein the stabilized phosphate group is 5′-vinyl phosphonate, optionally E-5′-vinyl phosphonate.
Embodiment 165. The RNAi agent of embodiment 163, wherein the stabilized phosphate group is 5′-cyclopropyl phosphonate.
Embodiment 166. The RNAi agent of any of embodiments 102-165, wherein the antisense RNAi oligonucleotide consists of 21-23, optionally 23, linked nucleosides.
Embodiment 167. The RNAi agent of any of embodiments 102-166, wherein each nucleoside in the sense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside.
Embodiment 168. The RNAi agent of embodiment 167, wherein each bicyclic nucleoside is independently selected from an LNA nucleoside, a cEt nucleoside, and an ENA nucleoside.
Embodiment 169. The RNAi agent of any of embodiments 102-168, wherein each nucleoside in the sense RNAi oligonucleotide is a stereo-standard nucleoside independently selected from: a 2′-OMe nucleoside, a 2′-F nucleoside, a 2′-MOE nucleoside, and a 2′-deoxynucleoside.
Embodiment 170. The RNAi agent of any of embodiments 102-169, wherein at least one nucleoside in the sense RNAi oligonucleotide is a 2′-OMe nucleoside.
Embodiment 171. The RNAi agent of embodiment 170, wherein 8-21 nucleosides in the sense RNAi oligonucleotide are 2′-OMe nucleosides, optionally wherein 13-21 nucleosides in the sense RNAi oligonucleotide are 2′-OMe nucleosides.
Embodiment 172. The RNAi agent of any of embodiments 102-171, wherein at least one of the nucleosides at positions 3-6 from the 5′-terminus of the sense RNAi oligonucleotide are 2′-OMe nucleosides, optionally wherein each of the nucleosides at positions 3-8 is a 2′-OMe nucleoside.
Embodiment 173. The RNAi agent of any of embodiments 102-172, wherein at least one of the nucleosides at positions 12-19 from the 5′-terminus of the sense RNAi oligonucleotide are 2′-OMe nucleosides, optionally wherein each of the nucleosides at positions 12-19 is a 2′-OMe nucleoside.
Embodiment 174. The RNAi agent of any of embodiments 102-173, wherein at least one nucleoside in the sense RNAi oligonucleotide is a 2′-F nucleoside.
Embodiment 175. The RNAi agent of embodiment 174, wherein 1-11 nucleosides in the sense RNAi oligonucleotide are 2′-F nucleosides, optionally wherein 2-4 nucleosides in the sense RNAi oligonucleotide are 2′-F nucleosides, optionally wherein exactly 2, 3, or 4 nucleosides in the sense RNAi oligonucleotide are 2′-F nucleosides.
Embodiment 176. The RNAi agent of any of embodiments 102-175, wherein one, two, three, or four of the nucleosides at positions 7 and 9-11 from the 5′-terminus of the sense RNAi oligonucleotide are 2′-F nucleosides, optionally wherein each of the nucleosides at positions 7 and 9-11 is a 2′-F nucleoside.
Embodiment 177. The RNAi agent of any of embodiments 102-176, wherein at least one nucleoside in the sense RNAi oligonucleotide is a 2′-deoxynucleoside.
Embodiment 178. The RNAi agent of any of embodiments 102-177, wherein one, two, three, four, or five of the nucleosides at positions 7-11 from the 5′-terminus of the sense RNAi oligonucleotide are 2′-deoxynucleosides, optionally wherein one of the nucleosides at positions 7-11 is a 2′-deoxynucleoside.
Embodiment 179. The RNAi agent of any of embodiments 102-178, wherein at least one nucleoside in the sense RNAi oligonucleotide is a 2′-MOE nucleoside.
Embodiment 180. The RNAi agent of embodiment 179, wherein 1-6 nucleosides in the sense RNAi oligonucleotide are 2′-MOE nucleosides, optionally wherein 2-4 nucleosides in the sense RNAi oligonucleotide are 2′-MOE nucleosides.
Embodiment 181. The RNAi agent of any of embodiments 102-180, wherein one, two, three, or four of the nucleosides at positions 1, 2, 20, and 21 from the 5′-terminus of the antisense RNAi oligonucleotide are 2′-MOE nucleosides, optionally wherein each of positions 1, 2, 20, and 21 is a 2′-MOE nucleoside.
Embodiment 182. The RNAi agent of any of embodiments 102-181, wherein one, two, three, or four of the nucleosides at positions 1, 2, 18, 19 from the 5′-terminus of the antisense RNAi oligonucleotide are 2′-MOE nucleosides, optionally wherein each of positions 1, 2, 18, and 19 is a 2′-MOE nucleoside.
Embodiment 183. The RNAi agent of any of embodiments 102-182, wherein at least one nucleoside in the sense RNAi oligonucleotide is substituted at its 2′-position with a C₁₆-C₂₂ alkyl group, optionally wherein the C₁₆-C₂₂ alkyl group is unsubstituted, optionally wherein the C₁₆-C₂₂ alkyl group is saturated.
Embodiment 184. The RNAi agent of any of embodiments 102-183, wherein the RNAi agent comprises a hairpin.
Embodiment 185. The RNAi agent of any of embodiments 102-184, wherein the RNAi agent comprises a tetraloop hairpin.
Embodiment 186. A population of RNAi agents of any of the preceding embodiments, wherein the stereo-non-standard nucleoside is chirally enriched for internucleoside linkages having the Rp or Sp configuration.
Embodiment 187. A population of RNAi agents of any of embodiments 1-185, wherein the stereochemical configuration at each internucleoside linkage is stereorandom.
Embodiment 188. A pharmaceutical composition comprising the RNAi agent or population of any of embodiments 1-187 and a pharmaceutically acceptable carrier or diluent.
Embodiment 189. A method comprising contacting a cell with the RNAi agent or population of any of embodiments 1-187 or pharmaceutical composition of embodiment 188.
Embodiment 190. A method of modulating the amount or activity of a target nucleic acid in a cell, comprising contacting the cell with the RNAi agent or population of any of embodiments 1-187 or pharmaceutical composition of embodiment 188.
Embodiment 191. The method of embodiment 190, wherein the amount or activity of a target nucleic acid is reduced.
Embodiment 192. Use of the RNAi agent, population, or composition of any of embodiments 1-188 for treatment of a disease or condition.
Embodiment 193. Use of the RNAi agent, population, or composition of any of embodiments 1-188 in therapy.
Embodiment 194. The RNAi agent, population, or composition of any of embodiments 1-188 for use in preparation of a medicament for treatment of a disease or condition.
Embodiment 195. The method or the use of any of embodiments 2-194, wherein the RNAi agent has reduced off-target effect compared to an RNAi agent having a stereo-standard nucleoside of the same 2′-substituent at the position of each nucleoside of Formula X.
Embodiment 196. The method or use of embodiment 195, wherein reduced off-target effect is determined by IC50 for knockdown of one or more off-target genes.
Embodiment 197. The method or use of embodiment 195, wherein reduced off-target effect is determined by number of differentially expressed off-target genes.

### Certain Compounds

In certain embodiments, described herein are oligomeric compounds (including oligomeric compounds that are RNAi agents or portions thereof) comprising a stereo-non-standard oligonucleoside.

### I. Stereo-Non-Standard Nucleosides

Certain stereo-non-standard nucleosides are described in WO2020/072991 and WO2021/030763, incorporated by reference herein in their entirety.

In certain embodiments, it was discovered that RNAi agents having a stereo-non-standard nucleoside within the seed region of its antisense oligonucleotide provided improved tolerability compared to an analogous RNAi agent lacking the stereo-non-standard nucleoside and having a stereo-standard nucleoside in the same position.

### II. Oligonucleotides

In certain embodiments, provided herein is an oligomeric compound comprising a modified antisense oligonucleotide comprising a stereo-non-standard nucleoside, and a modified sense oligonucleotide complementary to the modified antisense oligonucleotide. Certain modifications suitable for use in such oligomeric compounds are described below.

### A. Nucleosides

In certain embodiments, a modified sugar moiety is a non-bicyclic modified sugar moieties. In certain embodiments, modified sugar moieties are bicyclic or tricyclic sugar moieties. In certain embodiments, modified sugar moieties are sugar surrogates. Sugar surrogates may comprise one or more substitutions corresponding to those of other types of modified sugar moieties. In certain embodiments, a modified sugar moiety is a modified ribosyl sugar moiety. In some embodiments, a modified sugar moiety is a 2′-deoxyfuranosyl sugar moiety.

In certain embodiments, modified sugar moieties are non-bicyclic modified furanosyl sugar moieties comprising one or more substituent groups including, but not limited to, substituents at the 2′, 3′, 4′, and/or 5′ positions. In certain embodiments, the furanosyl sugar moiety is a ribosyl sugar moiety. In certain embodiments one or more non-bridging substituent of non-bicyclic modified sugar moieties is branched.

a. Substituted Sugar Moieties

In certain embodiments, a stereo-standard modified sugar moiety is a substituted furanosyl sugar moiety comprising one or more acyclic substituent, including but not limited to substituents at the 2′, 3′, 4′, and/or 5′ positions. In certain embodiments, the furanosyl sugar moiety is in the β-D-ribosyl configuration.

In certain embodiments, a non-bicyclic stereo-standard modified sugar moiety comprises a substituent group at the 2′-position. Examples of substituent groups include but are not limited to: 2′-F, 2′-OCH₃ (“OMe” or “O-methyl”), and 2′-O(CH₂)₂OCH₃ (“MOE” or “O-methoxyethyl”). In certain embodiments, 2′-substituent groups are selected from among: halo, allyl, amino, azido, SH, CN. OCN, CF₃. OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂₀N(Rₘ)(Rₙ) or OCH₂C(═O)—N(Rₘ)(Rₙ), where each Rₘ and Rₐ is, independently, H, an amino protecting group, or substituted or unsubstituted C₁-C₁₀ alkyl. —O(CH₂)₂ON(CH₃)₂(“DMAOE”), or 2′-O(CH₂)₂O(CH₂)₂N(CH₃)₂(“DMAEOE”). Synthetic methods for some of these 2′-substituent groups can be found. e.g., in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In certain embodiments, a 2′-substituted stereo-standard non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted stereo-standard sugar moiety of a modified nucleoside comprises 2′-substituent group selected from: F, OCF₃, OCH₃. OCH₂CH₂OCH₃, O(CH₂)₂SCH₃, O(CH₂)₂ON(CH₃)₂, O(CH₂)₂O(CH₂)₂N(CH₃)₂, O(CH₂)₂ON(CH₃)₂(“DMAOE”). O(CH₂)₂O(CH₂)₂N(CH₃)₂ (“DMAEOE”), and OCH₂C(═O)—N(H)CH₃ (“NMA”).

In certain embodiments, a 2′-substituted stereo-standard non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted stereo-standard sugar moiety of a modified nucleoside comprises a 2′-substituent group selected from: F, OCH₃, and OCH₂CH₂OCH₃.

In certain embodiments, non-bicyclic modified stereo-standard sugar moieties comprise a substituent group at the 4′-position. Examples of substituent groups suitable for the 4′-position of modified sugar moieties include, but are not limited to, alkoxy (e.g., methoxy), alkyl, and those described in Manoharan et al., WO 2015/106128.

In certain embodiments, non-bicyclic modified stereo-standard sugar moieties comprise a substituent group at the 3′-position. Examples of substituent groups suitable for the 3′-position of modified sugar moieties include, but are not limited to, alkoxy (e.g., methoxy), alkyl (e.g., methyl, ethyl).

In certain embodiments, non-bicyclic modified stereo-standard sugar moieties comprise a substituent group at the 5′-position. Examples of substituent groups suitable for the 5′-position of modified sugar moieties include, but are not limited to, vinyl, alkoxy (e.g., methoxy), and alkyl (e.g., methyl (R or S), ethyl).

In certain embodiments, non-bicyclic modified stereo-standard sugar moieties comprise more than one non-bridging sugar substituent, for example, 2′-F-5′-methyl sugar moieties, such as described in Migawa et al., US2010/0190837, or alternative 2′- and 5′-modified sugar moieties as described in Rajeev et al., US2013/0203836.

Certain modified sugar moieties comprise a substituent that bridges two atoms of the furanosyl ring to form a second ring, resulting in a bicyclic sugar moiety. In certain embodiments, the bicyclic sugar moiety comprises a bridge between the 4′ and the 2′ furanose ring atoms. Examples of such 4′ to 2′ bridging sugar substituents include, but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof, 4′-C(CH₃)(CH₃)—O-2′ and analogs thereof, 4′-CH₂—N(OCH₃)-2′ and analogs thereof, 4′-CH₂—O—N(CH₃)-2′. 4′-CH₂—C(H)(CH₃)-2′, 4′-CH₂—C(═CH₂)-2′ and analogs thereof), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₂ alkyl. Representative U.S. patents that teach the preparation of such bicyclic sugar moieties include, but are not limited to:Imanishi et al., U.S. Pat. No. 7,427,672; Swayze et al., U.S. Pat. No. 7,741,457, and Swayze et al., U.S. Pat. No. 8,022,193; Seth et al., U.S. Pat. No. 8,278,283; Prakash et al., U.S. Pat. No. 8,278,425; Seth et al., U.S. Pat. No. 8,278,426).

In certain embodiments, such 4′ to 2′ bridges independently comprise from 1 to 4 linked groups independently selected from: —[C(Ra)(Rb)]n-, —[C(Ra)(Rb)]n-O—, C(Ra)═C(Rb)—, C(Ra)=N—, C(═NRa)—, —C(═O)—, —C(═S)—, —O—, —Si(Ra)₂-, —S(═O)x-, and N(Ra)—;
wherein:
x is 0, 1, or 2;
n is 1, 2, 3, or 4;
   each Rₐ and R_{b} is, independently, H, a protecting group, hydroxyl, C1-C12 alkyl, substituted C1-C12 alkyl. C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl, or a protecting group.

Additional bicyclic sugar moieties are known in the art, see, for example: Wan, et al., J. Medicinal Chemistry, 2016, 59, 9645-9667; Wengel et al., U.S. Pat. No. 8,080,644; Ramasamy et al., U.S. Pat. No. 6,525,191; Seth et al., U.S. Pat. No. 7,547,684; and Seth et al., U.S. Pat. No. 7,666,854.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

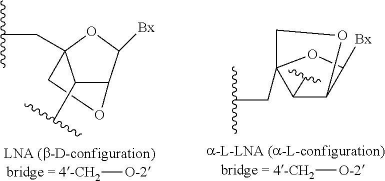

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the 1-D configuration, unless othenvise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the furanosyl oxygen atom of the sugar moiety may be replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2-position and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), and fluoro HNA (FHNA):altritolmannitolaltritol

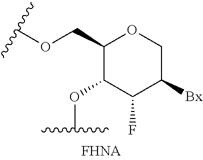

(“FHNA”, see e.g., Egli, et. al., J Am Chem Soc (2011) 133(41):16642-16649, Swayze et al., U.S. Pat. No. 8,088,904; and Swayze et al., U.S. Pat. No. 8,440,803); FHNA can also be referred to as a F-THP or 3′-fluoro tetrahydropyran or 3′-FHNA), and nucleosides comprising additional modified THP compounds having the formula:

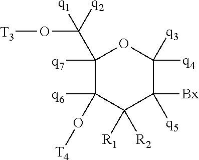

wherein, independently, for each of said modified THP nucleoside:
Bx is a nucleobase moiety;
T₃ and T₄ are each, independently, an internucleoside linkage, linking the modified THP nucleoside to the remainder of an oligonucleotide or one of T₃ and T₄ is an internucleoside linkage, linking the modified THP nucleoside to the remainder of an oligonucleotide and the other of T₃ and T₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl. C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments. R₁ is F and R₂ is H, in certain embodiments. R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported. As used here, the term “morpholino” means a sugar surrogate having the following structure:

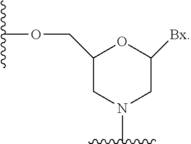

In certain embodiments, a morpholino is modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid, and nucleosides and oligonucleotides described in Manoharan et al., U.S. Pat. No. 10,913,767. Representative U.S. patents that teach the preparation of PNA compounds include, but are not limited to, U.S. Pat. Nos. 5,539,082; 5,714,331; and 5,719,262.

In certain embodiments, sugar surrogates are the “unlocked” sugar structure of UNA (unlocked nucleic acid) nucleosides. UNA is an unlocked acyclic nucleic acid, wherein any of the bonds of the sugar has been removed, fonning an unlocked sugar surrogate. Representative U.S. publications that teach the preparation of UNA include, but are not limited to, US Patent Publication No. 2011/0313020.

In certain embodiments, sugar surrogates are the glycerol as found in GNA (glycol nucleic acid) nucleosides as depicted below:

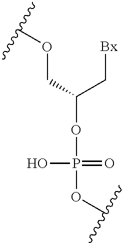

where Bx represents any nucleobase.

Many other bicyclic and tricyclic sugar and sugar surrogates are known in the art that can be used in modified nucleosides.

### 1. Alternative Linkage Positions

In naturally occurring nucleic acids, sugars are linked to one another 3′ to 5′. In certain embodiments, oligonucleotides include one or more nucleoside or sugar moiety linked at an alternative position, for example at the 2′ or inverted 5′ to 3′. For example, where the linkage is at the 2′ position, the 2′-substituent groups may instead be at the 3′-position.

### 2. Modified Nucleobases

In certain embodiments, a modified oligonucleotide comprises one or more nucleoside comprising an unmodified nucleobase. An “unmodified nucleobase” is unmodified adenine (A), unmodified thymine (T), unmodified cytosine (C), unmodified uracil (U), or unmodified guanine (G). In certain embodiments, a modified oligonucleotide comprises one or more inosine nucleosides (e.g., a nucleoside comprising a hypoxanthine nucleobase).

In certain embodiments, a modified oligonucleotide comprises one or more nucleoside that does not comprise a nucleobase, referred to as an abasic nucleoside.

In certain embodiments, a modified oligonucleotide comprises one or more nucleoside comprising a modified nucleobase. A modified nucleobase is a group of atoms other than unmodified A, T, C, U, or G capable of pairing with at least one other nucleobase. A 5-methylcytosine is an example of a modified nucleobase. A universal base is a modified nucleobase that can pair with any one of the five unmodified nucleobases.

Unless otherwise indicated, modified adenine has structure:

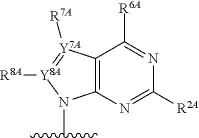

wherein: R^{2A} is H, C₁-C₆ alkyl, substituted C₁-C₆ alkyl, C₁-C₆ thioalkyl, or substituted C₁-C₆ thioalkyl, C₁-C₆ alkyloxy, or substituted C₁-C₆ alkyloxy: R^{A} is H, N(R^{a})(R^{b}), acetyl, formyl, or O-phenyl; Y^{8A} is N and R^{7A} is absent or is C₁-C₆ alkyl: or Y^{7A} is C and R^{7A} is selected from H. C₁-C₆ alkyl, or N(R^{a})(R^{b}); Y^{8A} is N and R^{8A} is absent, or Y^{8A} is C and R^{8A} is selected from H, a halogen, OH, C₁-C₆ alkyl, or substituted C₁-C₆ alkyl; R^{a} and R^{b} are independently selected from H, C₁-C₆ alkyl, substituted C₁-C₆ alkyl, C₁-C₆ alkenyl, substituted C₁-C₆ alkenyl, acetyl, formyl, or together form a 5-7-membered heterocycle; excluding where Y^{7A} is N; Y^{8A} is C, R^{8A} is H, R^{2A} is H, and R^{6A} is NH₂ (unmodified adenine).

Unless otherwise indicated, modified guanine has structure:

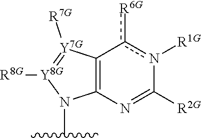

wherein: R^{2G} is N(R^{a})(R^{b}); R^{6G} is oxo and R^{1G} is H, or R^{6G} is selected from O—C₁-C₆ alkyl or S—C₁-C₆ alkyl and R^{1G} is absent: Y^{7G} is N and R^{7A} is absent or is C₁-C₆ alkyl; or Y^{7G} is C and R^{7G} is selected from H, C₁-C₆ alkyl, or N(Rₐ)(R_{b}) Y^{8G} is N and R^{8G} is absent, or Y^{8G} is C and R^{8G} is selected from H, a halogen, OH, C₁-C₆ alkyl, or substituted C₁-C₆ alkyl; R and R⁶ are independently selected from H, C₁-C₆ alkyl, substituted C₁-C₆ alkyl, C₁-C₆ alkenyl, substituted C₁-C₆ alkenyl, acetyl, formyl, or together form a 5-7-membered heterocycle; excluding where Y^{7G} is N; Y^{8G} is C. R^{8G} is H, R^{2G} is NH₂, and R^{6G} is =O (unmodified guanosine).

Unless otherwise indicated, modified thymine or modified uracil has structure:

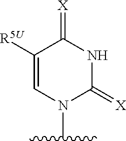

wherein: X is selected from O or S and R^{5U} is selected from H. OH, halogen, O—C₁-C₂₀ alkyl, O—C₁-C₁₂ substituted alkyl, C₁-C₁₂ alkyl, substituted C₁-C₁₂ alkyl, C₁-C₁₂ alkenyl, substituted C₁-C₁₂ alkenyl, C₁-C₁₂ alkynyl, substituted C₁-C₁₂ alkynyl; wherein if each X is O, R^{5U} is not H or CH₃ (unmodified uracil and unmodified thymine, respectively).

Unless otherwise indicated, modified cytosine has structure:

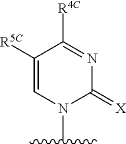

wherein: X is selected from O or S, R^{4C} is N(R^{a})(R^{b}); R^{5C} is selected from H, OH, halogen, O—C₁-C₂ alkyl, O—C₁-C₁₂ substituted alkyl, C₁-C₁₂ alkyl, substituted C₁-C₁₂ alkyl, C₁-C₁₂ alkenyl, substituted C₁-C₁₂ alkenyl; R and R^{b} are independently selected from H, C₁-C₆ alkyl, substituted C₁-C₆ alkyl, C₁-C₆ alkenyl, substituted C₁-C₆ alkenyl, C₁-C₁₂ alkynyl, substituted C,—C₁₂ alkynyl; acetyl, formyl, or together form a 5-7-membered heterocycle; excluding where X is O, R^{4C} is NH₂ and R^{5C} is H (unmodified cytosine).

In certain embodiments, modified nucleobases of a modified oligonucleotide are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 5-methylcytosine, 1-methylpseudouridine, 2-aminopropyladenine. 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo (particularly 5-bromo), 5-trifluoromethyl, 5-halouracil, and 5-halocytosine. 7-methylguanine, 7-methyladenine, 2-F-adenine. 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Englisch et al., Angewandte Chemie, International Edition, 1991, 30, 613; Sanghvi, Y.S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S.T., Ed., CRC Press, 2008, 163-166 and 442-443.

Publications that teach the preparation of certain of the above noted modified nucleobases, as well as other modified nucleobases include without limitation, Rogers et al., U.S. Pat. No. 5,134,066; Benner et al., U.S. Pat. No. 5,432,272; Matteucci et al., U.S. Pat. No. 5,502,177; Froehler et al., U.S. Pat. No. 5,594,121; and Cook et al., U.S. Pat. No. 5,681,941.

In certain embodiments, each nucleobase of a modified oligonucleotide is selected from unmodified A, ummodified G, unmodified C, unmodified T, unmodified U, and 5-methyl C.

In certain embodiments, there are no modified nucleobases in a modified oligonucleotide and each nucleobase of a modified oligonucleotide is selected from unmodified A, unmodified G, unmodified C, unmodified T, and unmodified U.

### 3. Modified Internucleoside Linkages

In certain embodiments, oligomeric compounds provided herein comprise or consist of a modified oligonucleotide comprising at least one modified internucleoside linkage. The naturally occurring internucleoside linkage found in RNA and DNA is a 3′ to 5′ phosphodiester linkage. In certain embodiments, nucleosides of modified oligonucleotides are linked together using one or more modified internucleoside linkages.

The two main classes of internucleoside linkages are defined by the presence or absence of a phosphorus atom. Representative phosphorus-containing internucleoside linkages include but are not limited to phosphates, which contain a phosphodiester bond (“P=O”) (also referred to as unmodified or naturally occurring linkages), phosphotriesters, methylphosphonates, phosphoramidates, and phosphorothioates (“P=S”), and phosphorodithioates (“HS—P=S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide.

In certain embodiments, a modified internucleoside linkage is any of those described in WO/2021/030778, incorporated by reference herein. In certain embodiments, a modified internucleoside linkage comprises the formula:

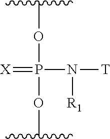

wherein independently for each internucleoside linkage of the modified oligonucleotide:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl. C₁-C₆ alkenyl, C₁-C₆ alkynyl, substituted C₁-C₆ alkyl, substituted C₁-C₆ alkenyl, substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

In certain embodiments, a modified internucleoside linkage comprises a mesyl phosphoramidate linkage having a formula:

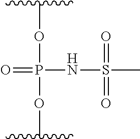 [SMILES: *OP(=O)(NS(C)(=O)=O)O*]

Certain internucleoside linkages having reduced charge (referred to as “neutral internucleoside linkages”) have been described. Such neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y.S. Sanghvi and P.D. Cook, Eds., ACS Symposium Series 580: Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

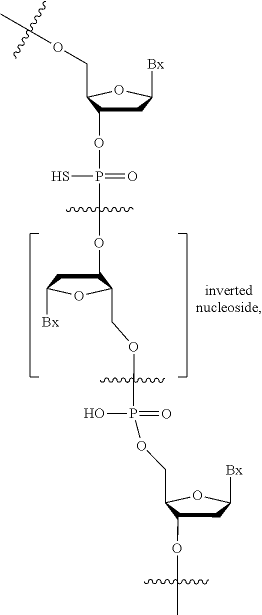

wherein each Bx independently represents any nucleobase.

In certain embodiments, an inverted nucleoside is terminal (i.e., the last nucleoside on one end of an oligonucleotide) and so only one internucleoside linkage depicted above will be present. In certain embodiments, additional features (e.g., a conjugate group) are attached to the inverted nucleoside. Such terminal inverted nucleosides can be attached to either or both ends of an oligonucleotide.

In certain embodiments, inverted nucleosides lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (e.g., a conjugate group) are attached to the inverted sugar moiety. A terminal inverted sugar moiety can be attached to either or both ends of an oligonucleotide.

In certain embodiments, nucleosides are linked 2′ to 5′ rather than the standard 3′ to 5′ linkage. Such a linkage is illustrated below.

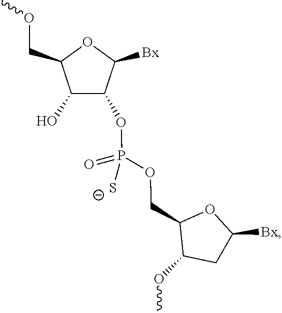

wherein each Bx represents any nucleobase.

In certain embodiments, internucleoside linkages have at least one chiral center. In such embodiments, a chiral atom can be prepared as a racemic mixture, or as separate enantiomers. Representative internucleoside linkages having a chiral center include but are not limited to alkylphosphonates, mesyl phosphoramidates, and phosphorothioates.

The mesyl phosphoramidate internucleoside linkage comprises a chiral center. In certain embodiments, modified oligonucleotides comprising (Rp) and/or (Sp) mesyl phosphoranidates comprise one or more of the following formulas, respectively, wherein “B” indicates a nucleobase:

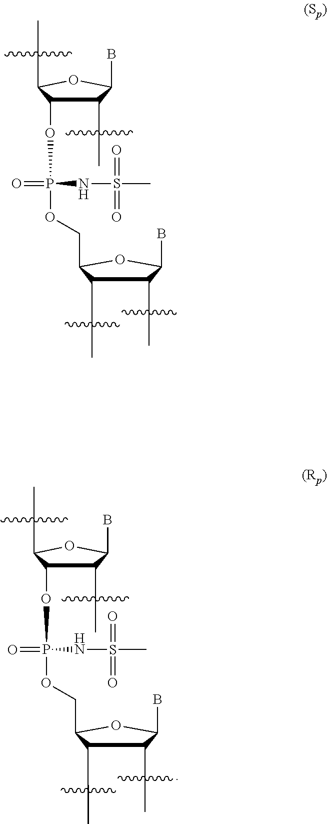

The phosphorothioate internucleoside linkage comprises a chiral center. In certain embodiments, modified oligonucleotides comprising (Rp) and/or (Sp) phosphorothioates comprise one or more of the following formulas, respectively, wherein “B” indicates a nucleobase:

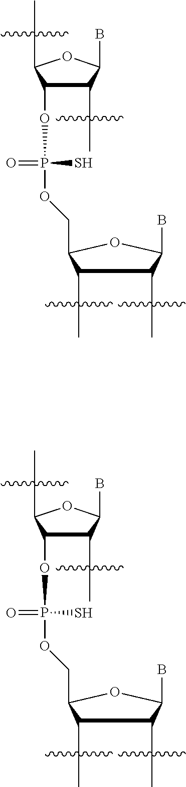

Modified oligonucleotides comprising internucleoside linkages having a chiral center can be prepared as populations of modified oligonucleotides comprising stereorandom internucleoside linkages, or as populations of modified oligonucleotides comprising linkages containing chiral centers in particular stereochemical configurations. In certain embodiments, populations of modified oligonucleotides comprise one or more phosphorothioate internucleoside linkages wherein all of the phosphorothioate internucleoside linkages are stereorandom. In certain embodiments, populations of modified oligonucleotides comprise one or more mesyl phosphoramidate internucleoside linkages wherein all of the mesyl phosphoramidate internucleoside linkages are stereorandom. Such modified oligonucleotides can be generated using synthetic methods that result in random selection of the stereochemical configuration of each phosphorothioate and/or mesyl phosphoramidate linkage. Nonetheless, each individual phosphorothioate and/or mesyl phosphoramidate of each individual oligonucleotide molecule has a defined stereoconfiguration. In certain embodiments, populations of modified oligonucleotides are enriched for modified oligonucleotides comprising one or more particular phosphorothioate and/or mesyl phosphoramidate internucleoside linkages in a particular, independently selected stereochemical configuration. In certain embodiments, the particular configuration of the particular phosphorothioate and/or mesyl phosphoramidate linkage is present in at least 65%, 70%, 80%, 90%, or 95% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555.

As used herein. “chirally enriched” in reference to a population means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom as defined herein. Chirally enriched populations of molecules having multiple chiral centers within each molecule may contain one or more stereorandom chiral centers. In certain embodiments, the chirally enriched center is at the phosphorous atom of a phosphorothioate internucleoside linkage, which may be in the Sp or Rp configuration. In certain embodiments, the chiral center is at the phosphorous atom of a mesyl phosphoramidate internucleoside linkage, which may be in the Sp or Rp configuration.

### B. Motifs

A modified oligonucleotides such as an antisense RNAi oligonucleotide or a sense RNAi oligonucleotide can be described by their motif, e.g. a pattern of unmodified and/or modified sugar moieties, nucleobases, and/or internucleoside linkages. In certain embodiments, the patterns or motifs of sugar moieties, nucleobases, and internucleoside linkages are each independent of one another. For example, where a patterns or motifs of sugar moieties or internucleoside linkages may be independent of nucleobase sequence. Thus, a modified oligonucleotide may be independently described by its sugar motif, nucleobase motif and/or internucleoside linkage motif Additionally, a sugar motif, nucleobase motif and/or internucleoside linkage motif may describe only a portion of a modified oligonucleotide.

### 1. Sugar Motifs

Provided herein is an antisense RNAi oligonucleotide comprising one or more stereo-non-standard nucleoside in the seed region thereof. Also provided is an RNAi agent comprising such an antisense RNAi oligonucleotide. In certain embodiments, the one or more stereo-non-standard nucleoside has a structure of Formula X. An RNAi agent of the disclosure may be an oligomeric duplex comprising an antisense RNAi oligomeric compound comprising an antisense RNAi oligonucleotide and a sense RNAi oligomeric compound comprising a sense RNAi oligonucleotide. Generally, the remainder of the nucleosides in the RNAi agent are stereo-standard nucleosides, however, an RNAi agent provided herein may comprise a (e.g., one or more) stereo-non-standard nucleoside outside the seed region of the antisense RNAi oligonucleotide, and/or may comprise a (e.g., one or more) sugar surrogate. Both the antisense RNAi oligonucleotide and the sense RNAi oligonucleotide are characterized by respective motifs of sugar moieties which can be conceptually separated from the nucleobase sequences thereof. The motif or sequence of sugar moieties is known to affect loading into protein complexes which are relevant to knockdown of a target mRNA or pre-mRNA. See. e.g., Hu, B., et al. Therapeutic siRNA: state of the art. Sig Transduct Target Ther 5, 101 (2020).

In certain embodiments, at least one nucleoside of a modified oligonucleotide comprises a 2′-OMe sugar moiety. In certain embodiments, at least 2, at least 5, at least 8, at least 10, at least 12, at least 13, at least 14, at least 15, at least 16, at least 17, at least 18, at least 19, at least 20, or at least 21 nucleosides comprise a 2′-OMe sugar moiety. In certain embodiments, a modified oligonucleotide comprises one, two, or three blocks of at least 4 contiguous 2′-OMe nucleosides. In certain embodiments, an antisense RNAi oligonucleotide comprises 2′-OMe nucleosides at nucleosides 3-5, counting from the 5′-terminal nucleoside. In certain embodiments, an antisense RNAi oligonucleotide comprises 2′-OMe nucleosides at nucleosides 7-11. In certain embodiments, an antisense RNAi oligonucleotide comprises 2′-OMe nucleosides at nucleosides 15 and 17-19. In certain embodiments, an antisense RNAi oligonucleotide comprises 2′-OMe nucleosides at nucleosides 3-13. In certain embodiments, each 2′-OMe nucleoside of a modified oligonucleotide comprises is a stereo-standard nucleoside. In certain embodiments, all but one 2′-OMe nucleoside of a modified oligonucleotide is a stereo-standard nucleoside. In certain such embodiments the remainder of the nucleosides, aside from stereo-non-standard nucleosides, in the modified oligonucleotide are selected from 2′-deoxynucleosides, 2′-F nucleosides, 2′-MOE nucleosides, and 2′-OMe nucleosides.

In certain embodiments, at least one nucleoside of a modified oligonucleotide comprises a 2′-F sugar moiety (i.e., a 2′-F modified nucleoside). In certain embodiments, a modified oligonucleotide comprises exactly 1, 2, 3, 4, or 5 nucleosides comprising a 2′-F sugar moiety. In certain embodiments, a modified oligonucleotide comprises a block of 2, 3, or 2-4 contiguous 2′-F nucleosides. In certain embodiments, an antisense RNAi oligonucleotide comprises a 2′-F nucleoside at one, two, three, or four of nucleosides 2, 6, 14, and/or 16, counting from the 5′-terminal nucleoside. In certain embodiments, an antisense RNAi oligonucleotide comprises a 2′-F nucleoside at one, two, or three of nucleosides 2, 14, and/or 16. In certain embodiments, an antisense RNAi oligonucleotide comprises a 2′-F nucleoside at one or two of nucleosides 2 and/or 14. In certain such embodiments the remainder of the nucleosides in the modified oligonucleotide, aside from stereo-non-standard nucleosides, are selected from 2′-deoxy nucleosides. 2′-MOE nucleosides, and 2′-OMe nucleosides. In certain such embodiments the remainder of the nucleosides in the modified oligonucleotide are stereo-standard nucleoside2′-OMe nucleosides.

In certain embodiments, each 2′-F nucleoside of a modified oligonucleotide is a stereo-standard 2′-F nucleoside. In certain embodiments, all but one 2′-F nucleoside of a modified oligonucleotide is a stereo-standard 2′-F nucleoside. In any of the embodiments described herein, a modified oligonucleotide may comprise a nucleoside comprising an FHNA sugar surrogate. In any of the embodiments described herein comprising a 2′-F nucleoside, one, two, three, one or more, or all such 2′-F nucleoside may be replaced with a nucleoside comprising an FHNA sugar surrogate.

In certain embodiments, the modified oligonucleotide comprises 1, 2, or 3 2′-deoxy sugar moieties. In certain embodiments, each 2′-deoxynucleoside of a modified oligonucleotide comprises a 2′-β-D-deoxyribosyl sugar moiety. In certain embodiments, all but one 2′-deoxynucleoside nucleoside of a modified oligonucleotide comprises a 2′-β-D-deoxyribosyl sugar moiety. In certain embodiments, an antisense RNAi oligonucleotide comprises a 2′-deoxy nucleoside at one or two of nucleosides 5, 6, and 7, counting from the 5′-terminal nucleoside. In certain embodiments, an antisense RNAi oligonucleotide comprises a 2′-deoxy nucleoside at nucleoside 6. In certain embodiments, an antisense RNAi oligonucleotide comprises a 2′-deoxy nucleoside at nucleosides 5 and 7. In certain such embodiments the remainder of the nucleosides, aside from stereo-non-standard nucleosides, in the modified oligonucleotide are selected from 2′-F nucleosides, 2′-MOE nucleosides, and 2′-OMe nucleosides.

In certain embodiments, the modified oligonucleotide comprises 1, 2, 3, 4 or 5 2′-MOE sugar moieties. In certain embodiments, each 2′-MOE nucleoside of a modified oligonucleotide is a stereo-standard 2′-MOE nucleoside. In certain embodiments, all but one 2′-MOE nucleoside of a modified oligonucleotide is a stereo-standard 2′-MOE nucleoside. In certain embodiments, an antisense RNAi oligonucleotide comprises a 2′-MOE nucleoside at one, two, three, four, or five of nucleosides 1, 9, 10, 22, and 23, counting from the 5′-terminal nucleoside. In certain embodiments, an antisense RNAi oligonucleotide comprises a 2′-MOE nucleoside at nucleoside 1. In certain embodiments, an antisense RNAi oligonucleotide comprises a 2′-MOE nucleoside at one or two of nucleosides 9 and/or 10. In certain embodiments, an antisense RNAi oligonucleotide comprises a 2′-MOE nucleoside at one or two of nucleosides 22 and/or 23. In certain such embodiments the remainder of the nucleosides, aside from stereo-non-standard nucleosides, in the modified oligonucleotide are selected from 2′-deoxy nucleosides, 2′-F nucleosides, and 2′-OMe nucleosides.

Certain RNAi motifs are described in, e.g., Freier, et al., WO2020/160163, incorporated by reference herein in its entirety; as well as, e.g., Rajeev, et al., WO2013/075035; Maier, et al., WO2016/028649; Theile, et al., WO2018/098328; Nair, et al., WO2019/217459: each of which is incorporated by reference herein.

In certain embodiments, a sugar motif of the antisense RNAi oligonucleotide is selected from those described in WO 2022/174053, wherein one nucleoside in the seed region is replaced with a stereo-non-standard nucleoside as described herein. In certain embodiments, a sugar motif of the sense RNAi oligonucleotide is selected from those described in WO 2022/174053.

In certain embodiments, a modified oligonucleotide is a uniformly modified oligonucleotide in which each modified nucleoside comprises the same 2′-modification. In certain embodiments, every second nucleoside of a uniformly modified nucleotide comprises the same 2′-modification, providing alternating 2′-modifications. In certain such embodiments, the 2′ modifications are 2′-OMe and 2′-F (i.e., alternating 2′-OMe and 2′-F nucleosides).

### 3. Internucleoside Linkage Motifs

In certain embodiments, the 5′-most linkage of the modified oligonucleotide (i.e., linking the first nucleoside from the 5′-end to the second nucleoside from the 5′-end) is modified. In certain embodiments, the two 5′-most linkages are modified. In certain embodiments, the first one or 2 linkages from the 3′-end are modified. In certain embodiments, the modified linkage is a phosphorothioate linkage. In certain embodiments, the modified linkage is a mesyl phosphoramidate linkage. In certain embodiments, the remaining linkages are all unmodified phosphodiester linkages.

In certain embodiments an antisense oligonucleotide has or comprises an internucleoside linkage motif (from 5′ to 3′) of: ssooooooooooooooooooss, wherein each ‘o’ represents a phosphodiester internucleoside linkage and each ‘s’ represents a phosphorothioate internucleoside linkage. In certain embodiments an antisense oligonucleotide has or comprises an internucleoside linkage motif (from 5′ to 3′) of: ssooooooooooooooooss, wherein each ‘o’ represents a phosphodiester internucleoside linkage and each ‘s’ represents a phosphorothioate internucleoside linkage.

In certain embodiments a sense oligonucleotide has an internucleoside linkage motif (from 5′ to 3′) of: ssooooooooooooooooss, wherein each ‘o’ represents a phosphodiester internucleoside linkage and each ‘s’, represents a phosphorothioate internucleoside linkage. In certain embodiments a sense oligonucleotide has an internucleoside linkage motif (from 5′ to 3′) of: ssooooooooooooooss, wherein each ‘o’ represents a phosphodiester internucleoside linkage and each ‘s’ represents a phosphorothioate internucleoside linkage.

### C. Lengths

It is possible to increase or decrease the length of an oligonucleotide without eliminating activity. For example, in Woolf ct al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, antisense oligonucleotides consist of 19-24 linked nucleosides. In certain embodiments, antisense oligonucleotides consist of 21-23 linked nucleosides. In certain embodiments, antisense oligonucleotides consist of 19 linked nucleosides. In certain embodiments, antisense oligonucleotides consist of 20 linked nucleosides. In certain embodiments, antisense oligonucleotides consist of 21 linked nucleosides. In certain embodiments, antisense oligonucleotides consist of 22 linked nucleosides. In certain embodiments, antisense oligonucleotides consist of 23 linked nucleosides.

In certain embodiments, sense oligonucleotides consist of 12-23 linked nucleosides. In certain embodiments, sense oligonucleotides consist of 18-25 linked nucleosides. In certain embodiments, sense oligonucleotides consist of 18-21 linked nucleosides. In certain embodiments, sense oligonucleotides consist of 19-21 linked nucleosides. In certain embodiments, sense oligonucleotides consist of 18 linked nucleosides. In certain embodiments, sense oligonucleotides consist of 19 linked nucleosides. In certain embodiments, sense oligonucleotides consist of 20 linked nucleosides. In certain embodiments, sense oligonucleotides consist of 21 linked nucleosides.

### D. Complementarity

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid, where the target nucleic acid may be a gene encoding a protein. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### III. Oligomeric Duplexes

In certain embodiments, an oligomeric compound provided herein comprises a modified oligonucleotide having a nucleobase sequence complementary to a sequence in a target nucleic acid paired with a second modified oligonucleotide having a region complementary to the first modified oligonucleotide to form an oligomeric duplex. In certain embodiments, the first oligomeric compound of an oligomeric duplex comprises or consists of (1) a first modified oligonucleotide and optionally a conjugate group and/or terminal group; and the second oligomeric compound of the oligomeric duplex comprises or consists of (2) a second modified oligonucleotide and optionally a terminal group and/or a conjugate group. Either or both oligomeric compounds of an oligomeric duplex may comprise a conjugate group or a terminal group. The oligonucleotides of each modified oligonucleotide of an oligomeric duplex may include non-complementary or unpaired overhanging nucleosides. In certain embodiments, the first and second modified oligonucleotides have at least one mismatch base pair relative to one another. In certain embodiments, the oligomeric duplex is an RNAi agent.

In certain embodiments, an oligomeric duplex comprises: a first oligomeric compound comprising a first modified oligonucleotide consisting of 15 to 30 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases; and a second oligomeric compound comprising a second modified oligonucleotide consisting of 15 to 29 linked nucleosides, wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or at least 21 contiguous nucleobases. In certain embodiments, the first oligomeric compound of the oligomeric duplex is an antisense compound, wherein the first modified oligonucleotide is an antisense oligonucleotide. In certain embodiments, the second oligomeric compound of the oligomeric duplex is a sense compound, wherein the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the nucleobase sequence of the second modified oligonucleotide is at least 90%, 95% or 100% complementary to the nucleobase sequence of an equal length portion of the first modified oligonucleotide. In certain embodiments, the nucleobase sequence of the second modified oligonucleotide comprises a complementary region of at least 9, at least 10, at least 11, at least 12, at least 13, at least 14, at least 15, at least 16, at least 17, at least 18, at least 19, at least 20, or 21 nucleobases that is 100% complementary to the nucleobase sequence of an equal portion of the first modified oligonucleotide.

In certain embodiments, the oligomeric duplex comprises unpaired nucleosides at either or both ends, forming one or two overhang ends. In certain embodiments an overhang end is one or two nucleosides of the antisense oligonucleotide. In certain embodiments an overhang end is one or two 3′-nucleosides of the antisense oligonucleotide.

In certain embodiments, in an oligomeric duplex provided herein, at least one nucleoside of the first modified oligonucleotide and/or the second modified oligonucleotide comprises a sugar surrogate. Examples of suitable sugar surrogates include, but are not limited to, morpholino, hexitol nucleic acid (HNA), fluoro-hexitol nucleic acid (FHNA), the sugar surrogates of glycol nucleic acid (GNA), and unlocked nucleic acid (UNA). In certain embodiments, at least one nucleoside of the first modified oligonucleotide comprises a FHNA. In certain embodiments, at least 80%, at least 90%, or 100% of the nucleosides of the first modified oligonucleotide and/or the second modified oligonucleotide comprises a modified stereo-standard sugar moiety and/or sugar surrogate selected from 2′-F, 2′-MOE, 2′-OMe, 2′-deoxyribosyl, and FHNA.

In certain embodiments, in an oligomeric duplex provided herein, the first modified oligonucleotide comprises a terminal group comprising a stabilized phosphate group attached to the 5′ position of the 5′-most nucleoside. In certain embodiments, the stabilized phosphate group comprises a cyclopropyl phosphonate or an (E)-vinyl phosphonate. In certain particular embodiments, the stabilized phosphate group is an (E)-vinyl phosphonate.

In certain embodiments, provided is an RNAi agent comprising an antisense RNAi oligomeric compound comprising an antisense RNAi oligonucleotide consisting of 20-50 linked nucleosides and optionally a sense RNAi oligomeric compound comprising a sense RNAi oligonucleotide consisting of 15-23 linked nucleosides, wherein the antisense RNAi oligonucleotide comprises a nucleoside having Formula I or Formula VII:

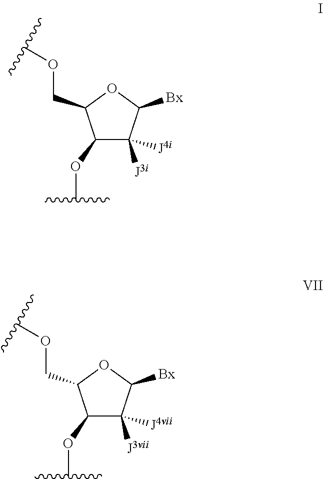

wherein J³ⁱ is H and J⁴ⁱ is selected from H, OH, OMe, F, and OCH₂CH₂OCH₃;
wherein J^{3vii} is H and J^{4vii} is selected from H, OH, OMe, F, and OCH₂CH₂OCH₃;
and Bx is a heterocyclic base moiety;
wherein nucleoside 5, 6, or 7 from the 5′-terminus of the antisense RNAi oligonucleotide is the nucleoside of Formula I or Formula VII.

In certain embodiments, the RNAi agent comprises a nucleoside having Formula I, J⁴ⁱ is H, and the remainder of the nucleosides in the RNAi agent are stereo-standard nucleosides.

In certain embodiments, the RNAi agent comprises a nucleoside having Formula VII, J^{4vii} is H, and the remainder of the nucleosides in the RNAi agent are stereo-standard nucleosides.

In certain embodiments, provided herein is an RNAi agent comprising an antisense RNAi oligomeric compound comprising an antisense RNAi oligonucleotide consisting of 21-23 linked nucleosides and a sense RNAi oligomeric compound comprising a sense RNAi oligonucleotide consisting of 14-21 linked nucleosides, wherein the antisense RNAi oligonucleotide comprises a nucleoside having Formula I:

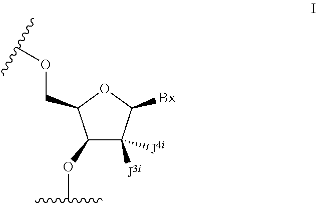

wherein J³ⁱ is H and J⁴ⁱ is H;
wherein Bx is selected from uracil, thymine, cytosine, 5-methyl cytosine, adenine, guanine, and hypoxanthine;
wherein nucleoside 5, 6, or 7 from the 5′-terminus of the antisense RNAi oligonucleotide is a nucleoside of Formula I, optionally wherein the remaining nucleosides in the antisense RNAi oligonucleotide are stereo-standard nucleosides;
wherein the wherein the 5′-terminal nucleoside of the antisense RNAi oligonucleotide is a 2′-MOE.

In certain embodiments, provided herein is an RNAi agent comprising an antisense RNAi oligomeric compound comprising an antisense RNAi oligonucleotide consisting of 21-23 linked nucleosides and a sense RNAi oligomeric compound comprising a sense RNAi oligonucleotide consisting of 14-21 linked nucleosides, wherein the antisense RNAi oligonucleotide comprises a nucleoside having Formula I:

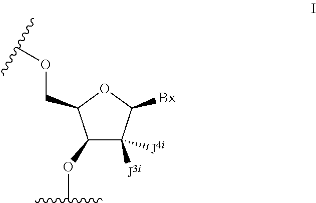

wherein J³ⁱ is H and J⁴ⁱ is H:
wherein Bx is selected from uracil, thymine, cytosine, adenine, guanine, and hypoxanthine;
wherein nucleoside 5, 6, or 7 from the 5′-terminus of the antisense RNAi oligonucleotide is a nucleoside of Formula I, optionally wherein the remaining nucleosides in the antisense RNAi oligonucleotide are stereo-standard nucleosides.

In certain embodiments of the RNAi agent, the remaining nucleosides in the antisense RNAi oligonucleotide are stereo-standard nucleosides.

In certain embodiments the RNAi agent is for treatment of a disease or condition in a subject, wherein the treatment comprises administering to a subject in need thereof a composition comprising the RNAi agent and a pharmaceutically acceptable carrier or diluent, optionally in a therapeutically effective amount thereof, optionally wherein the subject is a human.

In certain embodiments of the RNAi agent, the 5′-terminal nucleoside of the antisense RNAi oligonucleotide comprises a stabilized phosphate group.

In certain embodiments of the RNAi agent, Bx is selected from uracil, thymine, cytosine, 5-methyl cytosine, adenine, guanine, and hypoxanthine. In certain embodiments of the RNAi agent, Bx is selected from uracil, thymine, cytosine, adenine, guanine, and hypoxanthine. In certain embodiments of the RNAi agent, Bx is selected from uracil, thymine, cytosine, adenine, and guanine.

In certain embodiments of the RNAi agent, the nucleoside at position 1 from the 5′-terminus of the antisense RNAi oligonucleotide is a 2′-OMe or a 2′-MOE nucleoside.

In certain embodiments of the RNAi agent, one, two, or three of the nucleosides at positions 3, 4, and 5 from the 5′-terminus of the antisense RNAi oligonucleotide are 2′-OMe nucleosides.

In certain embodiments of the RNAi agent, each of the nucleosides at positions 3, 4, 5 from the 5′-terminus of the antisense RNAi oligonucleotide are 2′-OMc nucleosides.

In certain embodiments of the RNAi agent, one, two, three, four, or five of the nucleosides at positions 7, 8, 9, 10, and 11 from the 5′-terminus of the antisense RNAi oligonucleotide are 2′-OMe nucleosides.

In certain embodiments of the RNAi agent, one, two, three, four, or five of the nucleosides at positions 17, 18, 19, 20, and 21 from the 5′-terminus of the antisense RNAi oligonucleotide are 2′-OMe nucleosides.

In certain embodiments of the RNAi agent, each of the nucleosides at positions 17, 18, 19, 20, and 21 from the 5′-terminus of the antisense RNAi oligonucleotide are 2′-OMe nucleosides.

In certain embodiments of the RNAi agent, one, two, or three of the nucleosides at positions 2, 14, and 16 from the 5′-terminus of the antisense RNAi oligonucleotide are 2′-F nucleosides.

In certain embodiments of the RNAi agent, the nucleoside at position 6 from the 5′-terminus of the antisense RNAi oligonucleotide is a 2′-OMe nucleoside, a 2′-F nucleoside or a 2′-deoxynucleoside. In certain embodiments of the RNAi agent, the nucleoside at position 6 from the 5′-terminus of the antisense RNAi oligonucleotide is a 2′-F nucleoside. In certain embodiments of the RNAi agent, the nucleoside at position 6 from the 5′-terminus of the antisense RNAi oligonucleotide is a 2′-deoxy nucleoside. In certain embodiments of the RNAi agent, the nucleoside at position 6 from the 5′-terminus of the antisense RNAi oligonucleotide is a 2′-F nucleoside and the nucleoside of Formula I is at position 7 from the 5′-terminus of the antisense RNAi oligonucleotide. In certain embodiments of the RNAi agent, the nucleoside at position 6 from the 5′-terminus of the antisense RNAi oligonucleotide is a 2′-F nucleoside and the nucleoside of Formula I is at position 5 from the 5′-terminus of the antisense RNAi oligonucleotide. In certain embodiments of the RNAi agent, the nucleoside at position 6 from the 5′-terminus of the antisense RNAi oligonucleotide is a 2′-deoxy nucleoside and the nucleoside of Formula I is at position 5 from the 5′-terminus of the antisense RNAi oligonucleotide. In certain embodiments of the RNAi agent, the nucleoside at position 6 from the 5′-terminus of the antisense RNAi oligonucleotide is a 2′-deoxy nucleoside and the nucleoside of Formula I is at position 7 from the 5′-terminus of the antisense RNAi oligonucleotide.

In certain embodiments of the RNAi agent, one, two, or three of the nucleosides at positions 3, 4, and 5 from the 5′-terminus of the antisense RNAi oligonucleotide are 2′-deoxy nucleosides. In certain embodiments of the RNAi agent, the nucleoside at position 5 from the 5′-terminus of the antisense RNAi oligonucleotide is a 2′-deoxy nucleoside and the nucleoside of Formula I is at position 6 from the 5′-terminus of the antisense RNAi oligonucleotide. In certain embodiments of the RNAi agent, the nucleoside at position 7 from the 5′-terminus of the antisense RNAi oligonucleotide is a 2′-deoxy nucleoside and the nucleoside of Formula I is at position 6 from the 5′-terminus of the antisense RNAi oligonucleotide.

In certain embodiments of the RNAi agent, one, two, or three of the nucleosides at positions 7, 8, 9, 10, and 11 from the 5′-terminus of the antisense RNAi oligonucleotide are 2′-deoxy nucleosides.

In certain embodiments of the RNAi agent, one, two, or three of the nucleosides at positions 12, 13, 14, 15, and 16 from the 5′-terminus of the antisense RNAi oligonucleotide are 2′-deoxy nucleosides.

In certain embodiments, each 2′-deoxy nucleoside of the antisense RNAi oligonucleotide is linked at its 3′-position by a phosphorothioate internucleoside linkage.

In certain embodiments of the RNAi agent, each nucleoside in the sense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside.

In certain embodiments of the RNAi agent, at least one of the nucleosides at positions 3-6 from the 5′-terminus of the sense RNAi oligonucleotide are 2′-OMe nucleosides, optionally wherein each of the nucleosides at positions 3-8 is a 2′-OMe nucleoside.

In certain embodiments of the RNAi agent, at least one of the nucleosides at positions 12-19 from the 5′-terminus of the sense RNAi oligonucleotide are 2′-OMe nucleosides, optionally wherein each of the nucleosides at positions 12-19 is a 2′-OMe nucleoside.

In certain embodiments of the RNAi agent, one, two, three, or four of the nucleosides at positions 7 and 9-11 from the 5′-terminus of the sense RNAi oligonucleotide are 2′-F nucleosides, optionally wherein each of the nucleosides at positions 7 and 9-11 is a 2′-F nucleoside.

In certain embodiments of the RNAi agent, one, two, three, four, or five of the nucleosides at positions 7-11 from the 5′-terminus of the sense RNAi oligonucleotide are 2′-deoxynucleosides.

In certain embodiments, each 2′-deoxy nucleoside of the sense RNAi oligonucleotide is linked at its 3′-position by a phosphorothioate internucleoside linkage.

In certain embodiments of the RNAi agent, at least one nucleoside in the sense RNAi oligonucleotide is substituted at its 2′-position with a C16-C22 alkyl group, optionally wherein the C16-C22 alkyl group is unsubstituted, optionally wherein the C16-C22 alkyl group is saturated.

In certain embodiments, the RNAi agent comprises a conjugate linker and a conjugate moiety.

In certain embodiments, provided herein is an RNAi agent comprising an antisense RNAi oligomeric compound comprising an antisense RNAi oligonucleotide consisting of 21-23 linked nucleosides and a sense RNAi oligomeric compound comprising a sense RNAi oligonucleotide consisting of 14-21 linked nucleosides, wherein the antisense RNAi oligonucleotide comprises a nucleoside having Formula I:

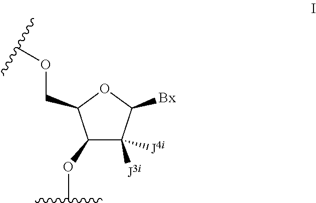

wherein J³ⁱ is H and J⁴ⁱ is selected from H, OH, OMe, F, and OCH₂CH₂OCH₃;
wherein Bx is selected from uracil, thymine, cytosine, 5-methyl cytosine, adenine, guanine, and hypoxanthine;
provided that the RNAi agent does not comprise any of the following:

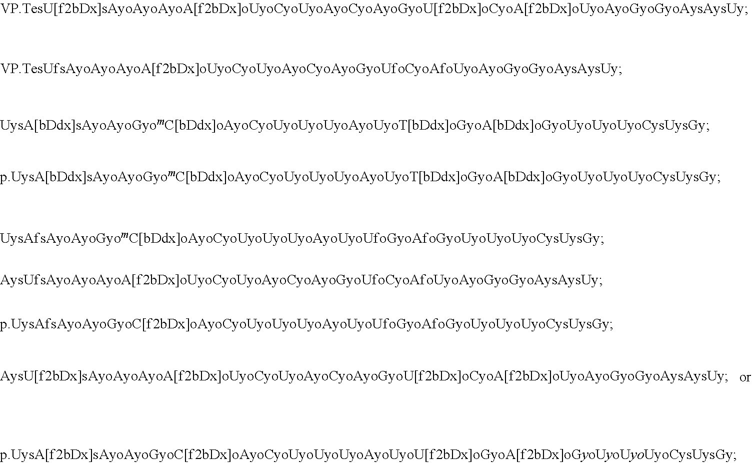

wherein “VP” represents a 5′-vinyl phosphonate moiety, “p” represents a 5′-phosphate moiety, a subscript “[f2bDx]” represents a 2-fluoro-β-D-xylosyl sugar moiety, a subscript “f” represents a 2′-fluoro-β-D-ribosyl sugar moiety, a subscript “y” represents a 2′-O-methyl-β-D-ribosyl sugar moiety, a subscript “e” represents a 2′-MOE-β-D-ribosyl sugar moiety, a superscript “m” represents a 5-methyl group on cytosine, a subscript “s” represents a phosphorothioate internucleoside linkage, and a subscript “o” represents a phosphodiester internucleoside linkage, optionally wherein J⁴ⁱ is H, wherein nucleoside 5, 6, or 7 from the 5′-terminus of the antisense RNAi oligonucleotide is a nucleoside of Formula I;
wherein the nucleoside at position 1 from the 5′-terminus of the antisense RNAi oligonucleotide is a bicyclic nucleoside, a 2′-O-alkyl nucleoside, a 2′-F nucleoside, a 2′-MOE nucleoside, a 2′-NMA nucleoside, a 2′-deoxynucleoside, a ribosyl nucleoside, a THP nucleoside, hexitol nucleic acid (“HNA”), altritol nucleic acid (“ANA”), mannitol nucleic acid (“MNA”), or a fluoro HNA (FHNA);
wherein the nucleoside at position 2 from the 5′-terminus of the antisense RNAi oligonucleotide is a 2′-F nucleoside or an FHNA;
wherein the nucleosides at positions 3 and 4 from the 5′-terminus of the antisense RNAi oligonucleotide are a bicyclic nucleoside, a 2′-O-alkyl nucleoside, a 2′-F nucleoside, a 2′-MOE nucleoside, a 2′-NMA nucleoside, a 2′-deoxynucleoside, a ribosyl nucleoside, a THP nucleoside, hexitol nucleic acid (“HNA”), altritol nucleic acid (“ANA”), mannitol nucleic acid (“MNA”), or a fluoro HNA;
wherein the nucleosides at positions 5, 6, and 7 from the 5′-terminus of the antisense RNAi oligonucleotide are a bicyclic nucleoside, a 2′-O-alkyl nucleoside, a 2′-F nucleoside, a 2′-MOE nucleoside, a 2′-NMA nucleoside, a 2′-deoxynucleoside, a ribosyl nucleoside, a THP nucleoside, hexitol nucleic acid (“HNA”), altritol nucleic acid (“ANA”), mannitol nucleic acid (“MNA”), or a fluoro HNA, or a nucleoside of formula I;
wherein the nucleosides at positions 8, 9, 10, and 11 from the 5′-terminus of the antisense RNAi oligonucleotide are a bicyclic nucleoside, a 2′-O-alkyl nucleoside, a 2′-F nucleoside, a 2′-MOE nucleoside, a 2′-NMA nucleoside, a 2′-deoxynucleoside, a ribosyl nucleoside, a THP nucleoside, hexitol nucleic acid (“HNA”), altritol nucleic acid (“ANA”), mannitol nucleic acid (“MNA”), or a fluoro HNA;
wherein the nucleosides at positions 14 and 16 from the 5′-terminus of the antisense RNAi oligonucleotide are a bicyclic nucleoside, a 2′-O-alkyl nucleoside, a 2′-F nucleoside, a 2′-MOE nucleoside, a 2′-NMA nucleoside, a 2′-deoxynucleoside, a ribosyl nucleoside, a THP nucleoside, hexitol nucleic acid (“HNA”), altritol nucleic acid (“ANA”), mannitol nucleic acid (“MNA”), or a fluoro HNA; optionally wherein the nucleosides at positions 14 and/or 16 from the 5′-terminus of the antisense RNAi oligonucleotide are 2′-F nucleosides;
wherein the nucleosides at positions 12, 13, and 15 from the 5′-terminus of the antisense RNAi oligonucleotide are a bicyclic nucleoside, a 2′-O-alkyl nucleoside, a 2′-F nucleoside, a 2′-MOE nucleoside, a 2′-NMA nucleoside, a 2′-deoxynucleoside, a ribosyl nucleoside, a THP nucleoside, hexitol nucleic acid (“HNA”), altritol nucleic acid (“ANA”), mannitol nucleic acid (“INA”), or a fluoro HNA;
wherein the nucleosides at positions 17, 18, 19, 20, and 21 from the 5′-terminus of the antisense RNAi oligonucleotide are a bicyclic nucleoside, a 2′-O-alkyl nucleoside, a 2′-F nucleoside, a 2′-MOE nucleoside, a 2′-NMA nucleoside, a 2′-deoxynucleoside, a ribosyl nucleoside, a THP nucleoside, hexitol nucleic acid (“HNA”), altritol nucleic acid (“ANA”), mannitol nucleic acid (“NINA”), or a fluoro HNA;
wherein the nucleosides at positions 22 and 23 from the 5′-terminus of the antisense RNAi oligonucleotide, if present, are a bicyclic nucleoside, a 2′-O-alkyl nucleoside, a 2′-F nucleoside, a 2′-MOE nucleoside, a 2′-NMA nucleoside, a 2′-deoxynucleoside, a ribosyl nucleoside, a THP nucleoside, hexitol nucleic acid (“HNA”), altritol nucleic acid (“ANA”), mannitol nucleic acid (“MNA”), or a fluoro HNA:
wherein the nucleosides at positions 1-2 from the 5′-terminus of the sense RNAi oligonucleotide are a bicyclic nucleoside, a 2′-O-alkyl nucleoside, a 2′-F nucleoside, a 2′-MOE nucleoside, a 2′-NMA nucleoside, a 2′-deoxynucleoside, a ribosyl nucleoside, a THP nucleoside, hexitol nucleic acid (“HNA”), altritol nucleic acid (“ANA”), mannitol nucleic acid (“MNA”), or a fluoro HNA;
wherein the nucleosides at positions 3-6 and 8 from the 5′-terminus of the sense RNAi oligonucleotide are a bicyclic nucleoside, a 2′-O-alkyl nucleoside, a 2′-F nucleoside, a 2′-MOE nucleoside, a 2′-NMA nucleoside, a 2′-deoxynucleoside, a ribosyl nucleoside, a THP nucleoside, hexitol nucleic acid (“HNA”), altritol nucleic acid (“ANA”), mannitol nucleic acid (“MNA”), or a fluoro HNA;
wherein the nucleosides at positions 7 and 9-11 from the 5′-terminus of the sense RNAi oligonucleotide are 2′-O-alkyl, 2′-deoxy, or 2′-F nucleosides, optionally wherein each of the nucleosides at positions 7 and 9-11 is a 2′-F nucleoside;
wherein the nucleosides at positions 12-19 from the 5′-terminus of the sense RNAi oligonucleotide are a bicyclic nucleoside, a 2′-O-alkyl nucleoside, a 2′-F nucleoside, a 2′-MOE nucleoside, a 2′-NMA nucleoside, a 2′-deoxynucleoside, a ribosyl nucleoside, a THP nucleoside, hexitol nucleic acid (“HNA”), altritol nucleic acid (“ANA”), mannitol nucleic acid (“MNA”), or a fluoro HNA; and
wherein the nucleosides at positions 20 and 21 from the 5′-terminus of the sense RNAi oligonucleotide, if present, are a bicyclic nucleoside, a 2′-O-alkyl nucleoside, a 2T-F nucleoside, a 2′-MOE nucleoside, a 2′-NMA nucleoside, a 2′-deoxynucleoside, a ribosyl nucleoside, a THP nucleoside, hexitol nucleic acid (“HNA”), altritol nucleic acid (“ANA”), mannitol nucleic acid (“MNA”), or a fluoro HNA;
optionally wherein a 2′-substituent of a sugar moiety is replaced with a conjugate group.

In certain embodiments, provided herein is an RNAi agent comprising an antisense RNAi oligomeric compound comprising an antisense RNAi oligonucleotide consisting of 21-23 linked nucleosides and a sense RNAi oligomeric compound comprising a sense RNAi oligonucleotide consisting of 14-21 linked nucleosides, wherein the antisense RNAi oligonucleotide comprises a nucleoside having Formula I:

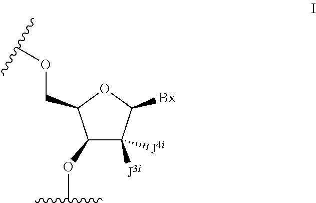

wherein J³ⁱ is H and J⁴ⁱ is selected from H, OH, OMe, F, and OCH₂CH₂OCH₃;
wherein Bx is selected from uracil, thymine, cytosine, 5-methyl cytosine, adenine, guanine, and hypoxanthine;
provided that the RNAi agent does not comprise any of the following:

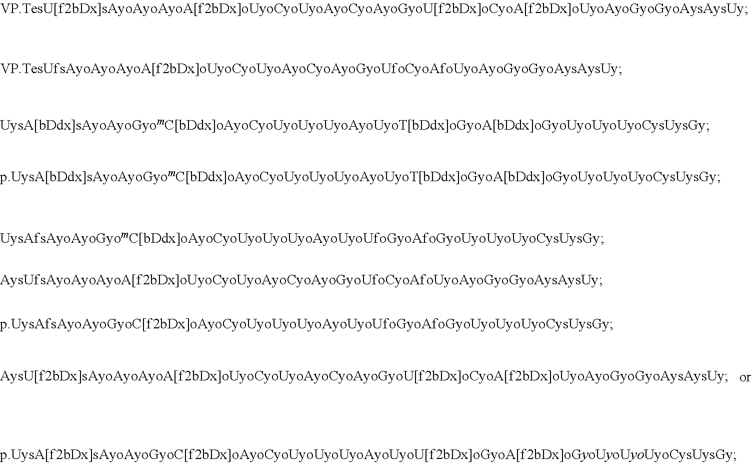

wherein “VP” represents a 5′-vinyl phosphonate moiety, “p” represents a 5′-phosphate moiety, a subscript “[f2bDx]” represents a 2′-fluoro-β-D-xylosyl sugar moiety, a subscript “f” represents a 2′-fluoro-β-D-ribosyl sugar moiety, a subscript “y” represents a 2′-O-methyl-β-D-ribosyl sugar moiety, a subscript “e” represents a 2′-MOE-β-D-ribosyl sugar moiety, a superscript “in” represents a 5-methyl group on cytosine, a subscript “s” represents a phosphorothioate internucleoside linkage, and a subscript “o” represents a phosphodiester internucleoside linkage, optionally wherein J⁴ⁱ is H, wherein nucleoside 5, 6, or 7 from the 5′-terminus of the antisense RNAi oligonucleotide is a nucleoside of Formula I;
wherein the nucleoside at position 1 from the 5′-terminus of the antisense RNAi oligonucleotide is a bicyclic nucleoside, a 2′-O-alkyl nucleoside, a 2′-F nucleoside, a 2′-MOE nucleoside, a 2′-NMA nucleoside, a 2′-deoxynucleoside, a ribosyl nucleoside, a THP nucleoside, hexitol nucleic acid (“HNA”), altritol nucleic acid (“ANA”), mannitol nucleic acid (“MNA”), or a fluoro HNA (FHNA);
wherein the nucleoside at position 2 from the 5′-terminus of the antisense RNAi oligonucleotide is a 2′-F nucleoside or an FHNA;
wherein the nucleosides at positions 3 and 4 from the 5′-terminus of the antisense RNAi oligonucleotide are 2′-OMe or 2′-F nucleosides;
wherein the nucleosides at positions 5, 6, and 7 from the 5′-terminus of the antisense RNAi oligonucleotide are 2′-OMe. 2′-F nucleosides. FHNA, or a nucleoside of formula I;
wherein the nucleosides at positions 8, 9, 10, and 11 from the 5′-terminus of the antisense RNAi oligonucleotide are 2′-OMe or 2′-F nucleosides;
wherein the nucleosides at positions 14 and 16 from the 5′-terminus of the antisense RNAi oligonucleotide are 2′-OMc, 2′-deoxy, 2′-F nuclcosides, or FHNA; optionally wherein the nucleosides at position 14 and/or 16 from the 5′-terminus of the antisense RNAi oligonucleotide are 2′-F nucleosides;
wherein the nucleosides at positions 12, 13, and 15 from the 5′-terminus of the antisense RNAi oligonucleotide are 2′-OMe or 2′-F nucleosides;
wherein the nucleosides at positions 17, 18, 19, 20, and 21 from the 5′-terminus of the antisense RNAi oligonucleotide are 2′-OMe or 2′-F nucleosides;
wherein the nucleosides at positions 22 and 23 from the 5-terminus of the antisense RNAi oligonucleotide, if present, are 2′-OMe, 2′-deoxy, or 2′-MOE nucleosides;
wherein the nucleosides at positions 1-2 from the 5′-terminus of the sense RNAi oligonucleotide are 2′-O-alkyl or 2′-MOE nucleosides;
wherein the nucleosides at positions 3-6 from the 5′-terminus of the sense RNAi oligonucleotide are 2′-O-alkyl or 2′-F nucleosides;
wherein the nucleosides at positions 7-11 from the 5′-terminus of the sense RNAi oligonucleotide are 2′-O-alkyl, 2′-deoxy, 2-F nucleosides, or FHNA, optionally wherein each of the nucleosides at positions 7 and 9-11 is a 2′-F nucleoside;
wherein the nucleosides at positions 12-19 from the 5′-terminus of the sense RNAi oligonucleotide are 2′-O-alkyl or 2′-F nucleosides; and
wherein the nucleosides at positions 20 and 21 from the 5′-terminus of the sense RNAi oligonucleotide, if present, are 2′-O-alkyl or 2′-MOE nucleosides.

In certain embodiments, provided herein is an RNAi agent comprising an antisense RNAi oligomeric compound comprising an antisense RNAi oligonucleotide consisting of 21-23 linked nucleosides and a sense RNAi oligomeric compound comprising a sense RNAi oligonucleotide consisting of 14-21 linked nucleosides, wherein the antisense RNAi oligonucleotide comprises a nucleoside having Formula I:

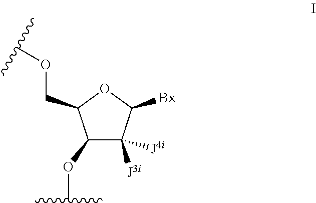

wherein J³ⁱ is H and J⁴ⁱ is selected from H, OH, OMe, F, and OCH₂CH₂OCH₃;
wherein Bx is selected from uracil, thymine, cytosine, 5-methyl cytosine, adenine, guanine, and hypoxanthine;
provided that the RNAi agent does not comprise any of the following:

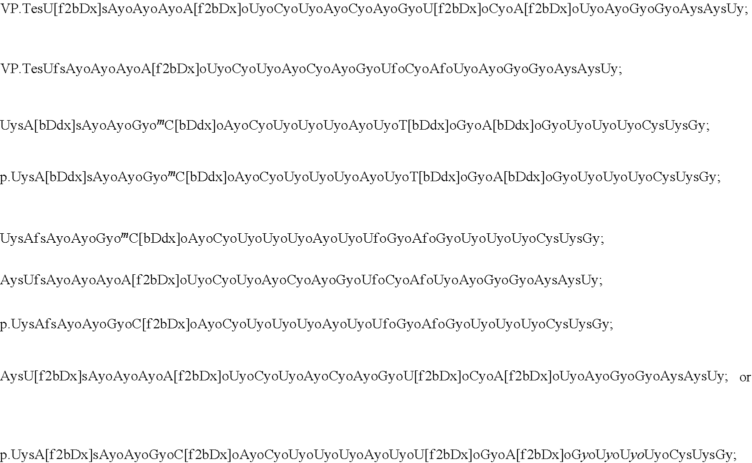

wherein “VP” represents a 5′-vinyl phosphonate moiety, “p” represents a 5′-phosphate moiety, a subscript “[f2bDx]” represents a 2-fluoro-β-D-xylosyl sugar moiety, a subscript “f” represents a 2′-fluoro-β-D-ribosyl sugar moiety, a subscript “y” represents a 2′-O-methyl-β-D-ribosyl sugar moiety, a subscript “e” represents a 2′-MOE-β-D-ribosyl sugar moiety, a superscript “m” represents a 5-methyl group on cytosine, a subscript “s” represents a phosphorothioate internucleoside linkage, and a subscript “o” represents a phosphodiester internucleoside linkage, optionally wherein J⁴ⁱ is H, wherein nucleoside 5, 6, or 7 from the 5′-terminus of the antisense RNAi oligonucleotide is a nucleoside of Formula I, wherein the remaining nucleosides in the antisense RNAi oligonucleotide are stereo-standard nucleosides, wherein the RNAi agent does not comprise a stereo-non-standard nucleoside outside of positions 3-8 from the 5′-terminus of the antisense RNAi oligonucleotide:
wherein the nucleoside at position 1 from the 5′-terminus of the antisense RNAi oligonucleotide is a 2′-OMe or 2′-MOE nucleoside;
wherein the nucleoside at position 2 from the 5′-terminus of the antisense RNAi oligonucleotide is a 2′-F nucleoside;
wherein the nucleosides at positions 3 and 4 from the 5′-terminus of the antisense RNAi oligonucleotide are 2′-OMe or 2′-F nucleosides:
wherein the nucleosides at positions 5, 6, and 7 from the 5′-terminus of the antisense RNAi oligonucleotide are 2′-OMe or 2′-F nucleosides, or a nucleoside of formula I;
wherein the nucleosides at positions 8, 9, 10, and 11 from the 5′-terminus of the antisense RNAi oligonucleotide are 2′-OMc or 2′-F nucleosides;
wherein the nucleosides at positions 14 and 16 from the 5-terminus of the antisense RNAi oligonucleotide are 2′-OMe. 2′-deoxy, or 2′-F nucleosides; optionally wherein the nucleosides at positions 14 and/or 16 from the 5′-terminus of the antisense RNAi oligonucleotide are 2′-F nucleosides;
wherein the nucleosides at positions 12, 13, and 15 from the 5′-terminus of the antisense RNAi oligonucleotide are 2′-OMe or 2′-F nucleosides;
wherein the nucleosides at positions 17, 18, 19, 20, and 21 from the 5′-terminus of the antisense RNAi oligonucleotide are 2′-OMe or 2′-F nucleosides;
wherein the nucleosides at positions 22 and 23 from the 5′-terminus of the antisense RNAi oligonucleotide, if present, are 2′-OMe or 2′-MOE nucleosides;
wherein the nucleosides at positions 1-2 from the 5′-terminus of the sense RNAi oligonucleotide are 2′-OMe or 2′-MOE nucleosides:
wherein the nucleosides at positions 3-6 and 8 from the 5′-terminus of the sense RNAi oligonucleotide are 2′-OMe or 2′-F nucleosides:
wherein the nucleosides at positions 7 and 9-11 from the 5′-terminus of the sense RNAi oligonucleotide are 2′-OMe, 2′-deoxy, or 2′-F nucleosides, optionally wherein each of the nucleosides at positions 7 and 9-11 is a 2′-F nucleoside;
wherein the nucleosides at positions 12-19 from the 5′-terminus of the sense RNAi oligonucleotide are 2′-OMe or 2′-F nucleosides; and
wherein the nucleosides at positions 20 and 21 from the 5′-terminus of the sense RNAi oligonucleotide, if present, are 2′-OMe or 2′-MOE nucleosides.

In certain embodiments, provided herein is an RNAi agent comprising an antisense RNAi oligomeric compound comprising an antisense RNAi oligonucleotide consisting of 21-23 linked nucleosides and a sense RNAi oligomeric compound comprising a sense RNAi oligonucleotide consisting of 14-21 linked nucleosides, wherein the antisense RNAi oligonucleotide comprises a nucleoside having Formula I:

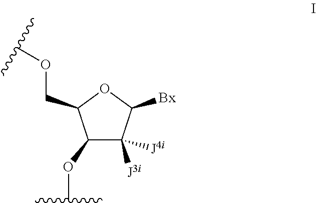

wherein J³ⁱ is H and J⁴ⁱ is H;
wherein Bx is selected from uracil, thymine, cytosine, adenine, guanine, and hypoxanthine;
wherein nucleoside 5, 6, or 7 from the 5′-terminus of the antisense RNAi oligonucleotide is a nucleoside of Formula I, optionally wherein the remaining nucleosides in the antisense RNAi oligonucleotide are stereo-standard nucleosides;
wherein the nucleoside at position 1 from the 5′-terminus of the antisense RNAi oligonucleotide is a 2′-OMe or 2′-MOE nucleoside;
wherein the nucleoside at position 2 from the 5′-terminus of the antisense RNAi oligonucleotide is a 2′-F nucleoside;
wherein the nucleosides at positions 3 and 4 from the 5′-terminus of the antisense RNAi oligonucleotide are 2′-OMe or 2′-F nucleosides;
wherein the nucleosides at positions 5, 6, and 7 from the 5′-terminus of the antisense RNAi oligonucleotide are 2′-OMe or 2′-F nucleosides, or a nucleoside of formula I;
wherein the nucleosides at positions 8, 9, 10, and 11 from the 5′-terminus of the antisense RNAi oligonucleotide are 2′-OMe or 2′-F nucleosides:
wherein the nucleosides at positions 14 and 16 from the 5′-terminus of the antisense RNAi oligonucleotide are 2′-OMe, 2′-deoxy, or 2′-F nucleosides; optionally wherein the nucleosides at positions 14 and/or 16 from the 5′-terminus of the antisense RNAi oligonucleotide are 2′-F nucleosides;
wherein the nucleosides at positions 12, 13, and 15 from the 5′-terminus of the antisense RNAi oligonucleotide are 2′-OMe or 2′-F nucleosides;
wherein the nucleosides at positions 17, 18, 19, 20, and 21 from the 5′-terminus of the antisense RNAi oligonucleotide are 2′-OMe or 2′-F nucleosides;
wherein the nucleosides at positions 22 and 23 from the 5′-terminus of the antisense RNAi oligonucleotide, if present, are 2′-OMe or 2′-MOE nucleosides;
wherein the nucleosides at positions 1-2 from the 5′-terminus of the sense RNAi oligonucleotide are 2′-OMe or 2′-MOE nucleosides;
wherein the nucleosides at positions 3-6 and 8 from the 5′-terminus of the sense RNAi oligonucleotide are 2′-OMe or 2′-F nucleosides;
wherein the nucleosides at positions 7 and 9-11 from the 5′-terminus of the sense RNAi oligonucleotide are 2′-OMe. 2′-deoxy, or 2′-F nucleosides, optionally wherein each of the nucleosides at positions 7 and 9-11 is a 2′-F nucleoside;
wherein the nucleosides at positions 12-19 from the 5′-terminus of the sense RNAi oligonucleotide are 2′-OMe or 2′-F nucleosides; and
wherein the nucleosides at positions 20 and 21 from the 5′-terminus of the sense RNAi oligonucleotide, if present, are 2′-OMe or 2′-MOE nucleosides.

In certain embodiments, provided is an RNAi agent comprising an antisense RNAi oligomeric compound comprising an antisense RNAi oligonucleotide consisting of 20-50 linked nucleosides and optionally a sense RNAi oligomeric compound comprising a sense RNAi oligonucleotide consisting of 15-23 linked nucleosides, wherein the antisense RNAi oligonucleotide comprises a nucleoside having Formula I:

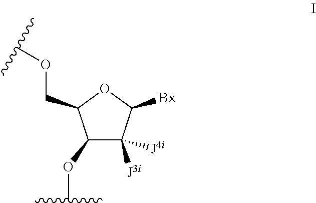

wherein J3i is H and J4i is selected from H, OH, OMe, F, and OCH2CH2OCH3;
and Bx is a heterocyclic base moiety;
provided that the RNAi agent does not comprise any of the following:

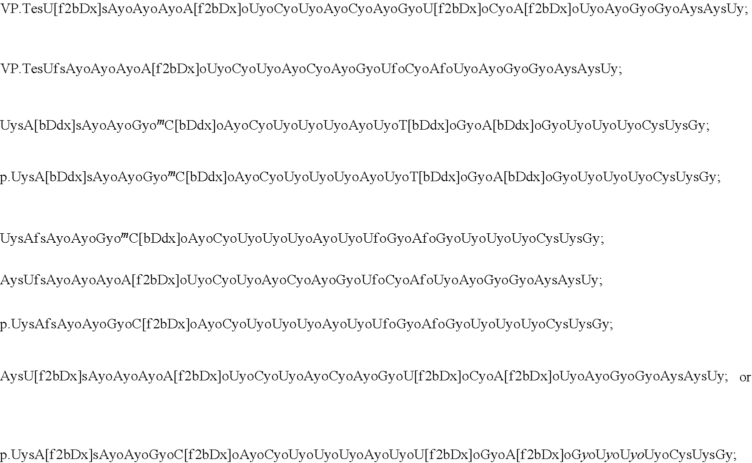

wherein “VP” represents a 5′-vinyl phosphonate moiety, “p” represents a 5′-phosphate moiety, a subscript “[f2bDx]” represents a 2′-fluoro-β-D-xylosyl sugar moiety, a subscript “f” represents a 2′-fluoro-β-D-ribosyl sugar moiety, a subscript “y” represents a 2′-O-methyl-β-D-ribosyl sugar moiety, a subscript “e” represents a 2′-MOE-β-D-ribosyl sugar moiety, a superscript “m” represents a 5-methyl group on cytosine, a subscript “s” represents a phosphorothioate internucleoside linkage, and a subscript “o” represents a phosphodiester internucleoside linkage;
wherein nucleoside 5, 6, or 7 from the 5′-terminus of the antisense RNAi oligonucleotide is a nucleoside of Formula I, optionally wherein the remaining nucleosides in the antisense RNAi oligonucleotide are stereo-standard nucleosides;
wherein each remaining nucleoside in the antisense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside;
optionally wherein the RNAi agent is for treatment of a disease or condition in a subject, wherein the treatment comprises administering to a subject in need thereof a composition comprising the RNAi agent and a pharmaceutically acceptable carrier or diluent, optionally in a therapeutically effective amount thereof, optionally wherein the subject is a human;
optionally wherein the 5′-terminal nucleoside of the antisense RNAi oligonucleotide comprises a stabilized phosphate group;
wherein Bx is selected from uracil, thymine, cytosine, 5-methyl cytosine, adenine, guanine, and hypoxanthine;
wherein the nucleoside at position 1 from the 5′-terminus of the antisense RNAi oligonucleotide is a 2′-OMe or a 2′-MOE nucleoside;
wherein one, two, or three of the nucleosides at positions 3, 4, and 5 from the 5′-terminus of the antisense RNAi oligonucleotide are 2′-OMe nucleosides;
wherein each of the nucleosides at positions 3, 4, 5 from the 5′-terminus of the antisense RNAi oligonucleotide are 2′-OMe nucleosides;
wherein one, two, three, four, or five of the nucleosides at positions 7, 8, 9, 10, and 11 from the 5′-terminus of the antisense RNAi oligonucleotide are 2′-OMe nucleosides;
wherein one, two, three, four, or five of the nucleosides at positions 17, 18, 19, 20, and 21 from the 5′-terminus of the antisense RNAi oligonucleotide are 2′-OMe nucleosides;
wherein each of the nucleosides at positions 17, 18, 19, 20, and 21 from the 5′-terminus of the antisense RNAi oligonucleotide are 2′-OMe nucleosides;
wherein one, two, or three of the nucleosides at positions 2, 14, and 16 from the 5′-terminus of the antisense RNAi oligonucleotide are 2′-F nucleosides;
wherein the nucleoside at position 6 from the 5′-terminus of the antisense RNAi oligonucleotide is a 2′-OMc nucleoside, a 2′-F nucleoside or a 2′-deoxynucleoside;
wherein each nucleoside in the sense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside;
wherein at least one of the nucleosides at positions 3-6 from the 5′-terminus of the sense RNAi oligonucleotide are 2′-OMe nucleosides, optionally wherein each of the nucleosides at positions 3-8 is a 2′-OMc nucleoside;
wherein at least one of the nucleosides at positions 12-19 from the 5′-terminus of the sense RNAi oligonucleotide are 2′-OMe nucleosides, optionally wherein each of the nucleosides at positions 12-19 is a 2′-OMe nucleoside;
wherein one, two, three, or four of the nucleosides at positions 7 and 9-11 from the 5′-terminus of the sense RNAi oligonucleotide are 2′-F nucleosides, optionally wherein each of the nucleosides at positions 7 and 9-11 is a 2′-F nucleoside;
optionally wherein one, two, three, four, or five of the nucleosides at positions 7-11 from the 5′-terminus of the sense RNAi oligonucleotide are 2′-deoxynucleosides; and
optionally wherein at least one nucleoside in the sense RNAi oligonucleotide is substituted at its 2′-position with a C16-C22 alkyl group, optionally wherein the C16-C22 alkyl group is unsubstituted, optionally wherein the C16-C22 alkyl group is saturated.

In certain embodiments, provided is an RNAi agent comprising an antisense RNAi oligomeric compound comprising an antisense RNAi oligonucleotide consisting of 20-50 linked nucleosides and optionally a sense RNAi oligomeric compound comprising a sense RNAi oligonucleotide consisting of 15-23 linked nucleosides, wherein the antisense RNAi oligonucleotide comprises a nucleoside having Formula VII:

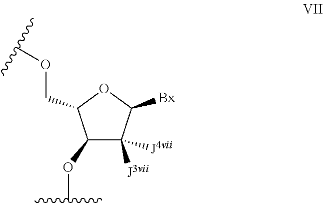

wherein J^{3vii} is H and J^{4vii} is selected from H. OH, OMe, F. and OCH₂CH₂OCH₃: optionally wherein J⁴ⁱⁱ is H;
and Bx is a heterocyclic base moiety;
wherein nucleoside 5, 6, or 7 from the 5′-terminus of the antisense RNAi oligonucleotide is a nucleoside of Formula VII, optionally wherein the remaining nucleosides in the antisense RNAi oligonucleotide are stereo-standard nucleosides:
optionally wherein the RNAi agent is for treatment of a disease or condition in a subject, wherein the treatment comprises administering to a subject in need thereof a composition comprising the RNAi agent and a pharmaceutically acceptable carrier or diluent, optionally in a therapeutically effective amount thereof, optionally wherein the subject is a human;
optionally wherein the 5′-terminal nucleoside of the antisense RNAi oligonucleotide comprises a stabilized phosphate group;
wherein Bx is selected from uracil, thymine, cytosine, 5-methyl cytosine, adenine, guanine, and hypoxanthine;
wherein the nucleoside at position 1 from the 5′-terminus of the antisense RNAi oligonucleotide is a 2′-OMe or a 2′-MOE nucleoside;
wherein one, two, or three of the nucleosides at positions 3, 4, and 5 from the 5′-terminus of the antisense RNAi oligonucleotide are 2′-OMe nucleosides;
wherein each of the nucleosides at positions 3, 4, 5 from the 5′-terminus of the antisense RNAi oligonucleotide are 2′-OMe nucleosides:
wherein one, two, three, four, or five of the nucleosides at positions 7, 8, 9, 10, and 11 from the 5′-terminus of the antisense RNAi oligonucleotide are 2′-OMe nucleosides;
wherein one, two, three, four, or five of the nucleosides at positions 17, 18, 19, 20, and 21 from the 5′-terminus of the antisense RNAi oligonucleotide are 2′-OMe nucleosides:
wherein each of the nucleosides at positions 17, 18, 19, 20, and 21 from the 5′-terminus of the antisense RNAi oligonucleotide are 2′-OMe nucleosides;
wherein one, two, or three of the nucleosides at positions 2, 14, and 16 from the 5′-terminus of the antisense RNAi oligonucleotide are 2′-F nucleosides;
wherein the nucleoside at position 6 from the 5′-terminus of the antisense RNAi oligonucleotide is a 2′-OMe nucleoside, a 2′-F nucleoside or a 2′-deoxynucleoside;
wherein each nucleoside in the sense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside;
wherein at least one of the nucleosides at positions 3-6 from the 5′-terminus of the sense RNAi oligonucleotide are 2′-OMe nucleosides, optionally wherein each of the nucleosides at positions 3-8 is a 2′-OMc nucleoside;
wherein at least one of the nucleosides at positions 12-19 from the 5′-terminus of the sense RNAi oligonucleotide are 2′-OMe nucleosides, optionally wherein each of the nucleosides at positions 12-19 is a 2′-OMe nucleoside;
wherein one, two, three, or four of the nucleosides at positions 7 and 9-11 from the 5′-terminus of the sense RNAi oligonucleotide are 2′-F nucleosides, optionally wherein each of the nucleosides at positions 7 and 9-11 is a 2′-F nucleoside;
optionally wherein one, two, three, four, or five of the nucleosides at positions 7-11 from the 5′-terminus of the sense RNAi oligonucleotide are 2′-deoxynucleosides; and
optionally wherein at least one nucleoside in the sense RNAi oligonucleotide is substituted at its 2′-position with a C16-C22 alkyl group, optionally wherein the C16-C22 alkyl group is unsubstituted, optionally wherein the C16-C22 alkyl group is saturated.

In certain embodiments, in an oligomeric duplex provided herein, the first or the second modified oligonucleotide is attached to a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide. In certain embodiments, the conjugate group is attached to the first modified oligonucleotide at an internal position. In certain embodiments, the conjugate group is attached to the first modified oligonucleotide through a 2′-modification of a furanosyl sugar moiety. In certain embodiments, the conjugate group is attached to the first modified oligonucleotide through a modified internucleoside linkage.

In certain embodiments, the conjugate group comprises N-acetyl galactosamine.

In certain embodiments, the conjugate group comprises a cell-targeting moiety having an affinity for transferrin receptor (TfR), TfR1, also known as CD71, TFRC. In certain embodiments, a conjugate group may comprises a moiety selected from any of a C22 alkyl, C20 alkyl, C16 alkyl. C10 alkyl, C21 alkyl, C19 alkyl, C18 alkyl. C17 alkyl, C15 alkyl. C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, a conjugate group comprises a moiety selected from any of C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

Certain isomers are also disclosed in WO 2014/022739, incorporated by reference herein in its entirety.

### IV. Conjugates

In certain embodiments, provided herein are compounds comprising one or more modified oligonucleotide and one or more conjugate groups. In certain embodiments, a compound optionally further comprises one or more terminal groups. In certain embodiments, compounds comprise an oligonucleotide, a cell-targeting moiety, and a conjugate linker. In certain embodiments, oligomeric compounds comprise an oligonucleotide, a transferrin receptor ligand, and a conjugate linker. In certain embodiments, oligomeric compounds comprise an oligonucleotide, a bicycle ligand, and a conjugate linker. In certain embodiments, oligomeric compounds comprise an oligonucleotide, a polypeptide, a conjugate linker, and optionally N-terminal or C-terminal modifications to the polypeptide. In certain embodiments, oligomeric compounds comprise an oligonucleotide, two or more polypeptides, a branching group, a conjugate linker, and optionally N-terminal or C-terminal modifications to the polypeptides. In certain embodiments, a conjugate linker connects a polypeptide and/or a bicycle ligand to an oligonucleotide.

In certain embodiments, the N-terminus of a bicycle ligand is covalently connected to a conjugate linker, and the conjugate linker is covalently connected to the 3′ end of an oligonucleotide. In certain embodiments, the C-terminus of a bicycle ligand is covalently connected to a conjugate linker, and the conjugate linker is covalently connected to the 3′ end of an oligonucleotide. In certain embodiments, an internal amino acid of a bicycle ligand is covalently connected to a conjugate linker, and the conjugate linker is covalently connected to the 3′ end of an oligonucleotide. In certain embodiments, the N-terminus of a bicycle ligand is covalently connected to a conjugate linker, and the conjugate linker is covalently connected to the 5′ end of an oligonucleotide. In certain embodiments, the C-terminus of a bicycle ligand is covalently connected to a conjugate linker, and the conjugate linker is covalently connected to the 5′ end of an oligonucleotide. In certain embodiments, an internal amino acid of a bicycle ligand is covalently connected to a conjugate linker, and the conjugate linker is covalently connected to the 5′ end of an oligonucleotide. In certain embodiments, the N-terminus of a bicycle ligand is covalently connected to a conjugate linker, and the conjugate linker is covalently connected at an internal position of an oligonucleotide. In certain embodiments, the C-terminus of a bicycle ligand is covalently connected to a conjugate linker, and the conjugate linker is covalently connected at an internal position of an oligonucleotide. In certain embodiments, an internal amino acid of a bicycle ligand is covalently connected to a conjugate linker, and the conjugate linker is covalently connected at an internal position of an oligonucleotide. In certain embodiments, an internal position of an oligonucleotide is a 2′-position of a modified sugar moiety of a nucleoside within the internal region of an oligonucleotide that is not the 5′ terminal nucleoside or the 3′ terminal nucleoside. In certain embodiments, an internal position of an oligonucleotide is a modified internucleoside linkage of the oligonucleotide.

### A. Conjugate Groups

In certain embodiments, a conjugate moiety modifies one or more properties of an attached oligonucleotide, including but not limited to pharmacodynamics, pharmacokinetics, stability, binding, absorption, tissue distribution, cellular distribution, cellular uptake, charge and clearance. In certain embodiments, a conjugate moiety imparts a new property on the attached oligonucleotide.

In certain embodiments, a conjugate group comprises a conjugate moiety and a conjugate linker. In certain embodiments, a conjugate moiety comprises or consists of a cell-targeting moiety. In certain embodiments, a cell-targeting moiety is capable of binding the cell-surface receptor or the cell-surface moiety. In certain embodiments, a compound comprising a cell-targeting moiety is capable of being internalized when it interacts with or binds the cell-surface receptor or the cell-surface moiety. In certain embodiments, a cell-targeting moiety comprises a bicyclic polypeptide or a bicycle ligand. In certain embodiments, a cell-targeting moiety consists of a bicyclic polypeptide or a bicycle ligand.

### Transferrin Receptor

In certain embodiments, a bicycle ligand is capable of interacting with the type 1 transferrin receptor. In certain embodiments, a bicycle ligand is capable of binding the type 1 transferrin receptor. In certain embodiments, a bicycle ligand is capable of binding the type 1 transferrin receptor while not interfering with the binding of the natural ligand transferrin. In certain embodiments, a bicycle ligand inhibits the binding of the natural ligand transferrin.

Cell-targeting moieties that have affinity for transferrin receptor (TfR1), including antibodies and antibody fragments, proteins, peptides, and aptamers, have been described. Such moieties may replace a bicycle conjugate described herein for targeting an oligomeric compound to cardiac cells (e.g., cardiac muscle cells)/tissue/heart. Thus, in certain embodiments, a conjugate group comprises a cell-targeting moiety that binds transferrin receptor (TfR). In certain embodiments, a conjugate group described herein comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the anti-TfR1 antibody or fragment thereof is any known in the art including but not limited to those described in WO1991/004753; WO2013/103800; WO2014/144060; WO2016/081643; WO2016/179257; WO2016/207240; WO2017/221883; WO2018/129384; WO2018/124121: WO2019/151539; WO2020/132584; WO2020/028864; U.S. Pat. Nos. 7,208,174; 9,034,329; and 10,550,188. In certain embodiments, a fragment of an anti-TfR1 antibody is F(ab′)2, Fab, Fab′, Fv, or scFv. In certain embodiments, the conjugate group comprises a protein or peptide capable of binding TfR1. In certain embodiments, the protein or peptide capable of binding TfR1 is any known in the art including but not limited to those described in WO2019/140050; WO2020/037150: WO2020/124032; and U.S. Pat. No. 10,138,483. In certain embodiments, the conjugate group comprises an aptamer capable of binding TfR1. In certain embodiments, the aptamer capable of binding TfR1 is any known in the art including but not limited to those described in WO2013/163303; WO2019/033051; and WO2020/245198. In certain embodiments, the conjugate group comprises a peptide, including but not limited to a cyclic peptide, capable of binding TfR1. In certain embodiments, the peptide capable of binding TfR1 is any known in the art including but not limited to those described in EP4108676: WO2023/027125; and WO2023/022234.

### Conjugate Linkers

In certain embodiments, oligomeric compounds comprise an oligonucleotide and a conjugate group, wherein the conjugate group comprises a conjugate moiety and a conjugate linker. In certain embodiments, the conjugate linker links the conjugate moiety to the oligonucleotide. In certain embodiments, the conjugate linker is a single chemical bond (i.e., the conjugate moiety is attached directly to an oligonucleotide through a single bond). In certain embodiments, the conjugate linker comprises one or more atoms. In certain embodiments, the conjugate linker comprises a chemical group. In certain embodiments, the conjugate linker comprises a chain structure, such as a hydrocarbyl chain, or an oligomer of repeating units such as ethylene glycol, nucleosides, or amino acid units. In certain embodiments, the oligonucleotide is a modified oligonucleotide. In certain embodiments, the conjugate moiety is a bicycle ligand. In certain embodiments, the conjugate moiety comprises two polypeptide loops attached to a molecular scaffold.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate moieties to parent compounds, such as the oligonucleotides provided herein. In general, a bifunctional linking moiety comprises at least two functional groups. One of the functional groups is selected to react with a particular site on a parent compound and the other is selected to react with a peptide extender. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

In certain embodiments, conjugate linkers comprise chemical groups that are formed upon a reaction between a first functional group and a second functional group. In certain embodiments, a modified oligonucleotide is attached to the first functional group during synthesis, and a conjugate moiety is attached to a second functional group during synthesis. Then, the two compounds are mixed under specific conditions to yield the final oligomeric compound. In certain embodiments, the conjugate moiety is a bicycle ligand. In certain embodiments, the conjugate moiety comprises two polypeptide loops attached to a molecular scaffold. Such reactions that are compatible with both oligonucleotide and peptide chemistry have been previously described and are often called “bioconjugation” reactions. These reactions include strain promoted azido-alkyne cycloaddition (SPAAC), copper-catalyzed click reaction (CuAAC), active ester conjugation to an amino modified oligonucleotide, maleimide-thiol Michael addition, ketol/hydroxylamine ligation, the Staudinger ligation, reductive amination, thio ether fonnation, disulfide formation, reductive alkylation, catalyst-free N-arylation, sulfur fluoride exchange click reaction (SuFEx), and inverse demand Diels Alder reaction. Certain such reactions are described in, e.g., Jbara, et al., “Oligonucleotide Bioconjugation with Bifunctional Palladium Reagents”, Angew. Chem. Int. Ed. 2021, 60(21)12109-12115; Dong, et al., “Sulfur(VI) Fluoride Exchange (SuFEx): Another Good Reaction for Click Chemistry,” Angew. Chem. Int. Ed. 2014, 53(36):9430-9448.4; Zhang, et al., “Arylation Chemistry for Bioconjugation,” Angew. Chem. Int. Ed. Engl. 2019; 58(15): 4810-4839; Walsh, et al., “Site-selective modification strategies in antibody-drug conjugates” Chem. Soc. Rev., 2021, 50: 1305-1353; Tiefenbrunn, et al., “Chemoselective ligation techniques: modern applications of time-honored chemistry”, Biopolymers, 2010, 94(1):95-106; Drake, et al., Bioconjug. Chem. 2014, 25(7):1331-1341: Bode, Acc. Chem. Res., 2017, 50, 9, 2104-2115; J. Magano, B. Bock, et al, Org. Proc. Res. Dev. 2014, 18:142-151; Craig S. McKay and M.G. Finn, “Click Chemistry in Complex Mixtures: Bioorthogonal Bioconjugation”, Chemistry & Biology 2014; Mitchell P. Christy et al., Org. Lett. 2020, 22: 2365; Ren et al., Angew. Chem. Int. Ed. Engl. 2009, 48, 9658-9662; Rohrbacher, F. et al., Helv. Chim. Acta. 2018, 101; Baalmaan, et al, “A Bioorthogonal Click Chemistry Toolbox for Targeted Synthesis of Branched and Well-Defined Protein-Protein Conjugates”, Angew. Chem. Int. Ed. 2020 (59): 12885-12893; Lang, et al, “Biorthogonal Reactions for Labeling Proteins”, J. Am. Chem. Soc, 2014, 9(1):16-20; Nair, et al., “The Thiol-Michael Addition Click Reaction: A Powerful and Widely Used Tool in Materials Chemistry”, Chem. Mater. 2013 26(1):724-744; Kalia and Raines, “Hydrolytic Stability of Hydrazones and Oximes”, Angew. Chem. Int. Ed., 2008, 47:7523-7526.

Examples of conjugate linkers include but are not limited to pyrrolidine. 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine. 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) an oligonucleotide consisting of 8-30 nucleosides and (2) a conjugate linker comprising 1-10 linker-nucleosides that are contiguous with the nucleosides of the oligonucleotide. The total number of contiguous linked nucleosides in such an oligomeric compound is more than 30. Alternatively, an oligomeric compound may comprise an oligonucleotide consisting of 8-30 nucleosides and no conjugate linker. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate moiety to be cleaved from the oligonucleotide. For example, in certain circumstances oligomeric compounds comprising a particular conjugate moiety are better taken up by a particular cell type, but once the oligomeric compound has been taken up, it is desirable that the conjugate moiety be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphodiester linkage between an oligonucleotide and a conjugate moiety.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

In certain embodiments, oligomeric compounds disclosed herein comprise an oligonucleotide linked to conjugate moiety by a conjugate linker, wherein the oligomeric compound is prepared using Click chemistry known in the art. Compounds have been prepared using Click chemistry wherein alkynyl phosphonate internucleoside linkages on an oligomeric compound attached to a solid support are converted into the 1,2,3-triazolylphosphonate internucleoside linkages and then cleaved from the solid support (Krishna et al., J. Am. Chem. Soc. 2012, 134(28), 11618-11631), which is incorporated by reference herein in its entirety. Additional conjugate linkers suitable for use in several embodiments is prepared by Click chemistry described in “Click Chemistry for Biotechnology and Materials Science” Ed. Joerg Laham, Wiley 2009, which is incorporated by reference herein in its entirety.

In certain embodiments, the linker is prepared by reaction of a first reactive moiety with a second reactive moiety, wherein the first reactive moiety is attached to the oligonucleotide and the second reactive moiety is attached to the conjugate moiety, or a precursor thereof. In certain embodiments, the linker is prepared by reaction of a dipolarophile (e.g., a triple bonded moiety such as an alkyne or nitrile) with a 1,3-dipole (e.g., an azide, a nitrone, an isocyanate, or a thioisocyanate):

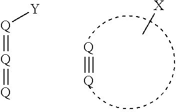

wherein each Q is independently a carbon atom or a heteroatom, for example, one of X and Y is attached to a cargo such as an oligonucleotide, and the other of X and Y is attached to a conjugate moiety such as a transferrin-receptor binding moiety or a half-life extension moiety. The linker thus prepared may comprise a five-membered unsaturated heterocyclic ring such as a triazole.

In certain embodiments, the linker is prepared by reaction of a dieneophile (e.g., an electron rich double bond such as a furan or derivative thereof) with an electron poor diene (e.g., a tetrazine):

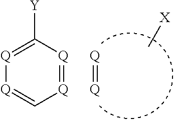

wherein each Q is independently a carbon atom or a heteroatom, for example, one of X and Y is attached to a cargo such as an oligonucleotide, and the other of X and Y is attached to a conjugate moiety such as a transferrin-receptor binding moiety or a half-life extension moiety. The linker thus prepared may comprise a six-membered unsaturated heterocyclic ring such as a dihydropyrazine.

In certain embodiments, the linker is prepared by reaction of a nucleophile (e.g., a thiol or amine) with an electrophile (e.g., an electron-poor carbonyl or carbonyl-conjugated alkene or alkyne):

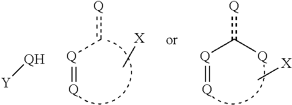

wherein each Q is independently a carbon atom or a heteroatom, for example, one of X and Y is attached to a cargo such as an oligonucleotide, and the other of X and Y is attached to a conjugate moiety such as a transferrin-receptor binding moiety or a half-life extension moiety. The linker thus prepared may comprise a thioether, hydrazone, oxime, or amide.

Each of the first reactive moiety and the second reactive moiety may attach at any suitable position of the modified oligonucleotide and the conjugate moiety. For example, at a 3′- or 5′-terminal position, or at a 2′-position of a furanosyl sugar moiety, to a nucleobase, to an internucleoside linkage, or to a sugar surrogate. In certain embodiments, the linker attaches at the 5′-terminal hydroxyl group of a modified oligonucleotide.

In certain embodiments, the conjugate linker comprises a pyrrolidine moiety. In certain embodiments, the pyrrolidine moiety has formula:

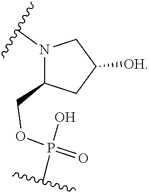 [SMILES: *N1C[C@H](O)C[C@H]1COP(*)(=O)O]

In certain embodiments, a linker has Formula L:

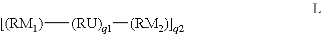

wherein each RM1 and RM2 is independently absent or a functional group derived from conjugation of a reactive moiety, for example wherein RM1 and RM2 are independently selected from an amino acid, O, NH, NR^{a2}, S—S, O—NH, O—NR^{a2}, NH—NH, NH—NR^{a2}, NR^{a2}—NR^{a2}, C₁₋₆ haloalkylene, C₂₋₆ alkenylene, C₂₋₆ alkynylene, C₃₋₁₀ cycloalkylene, C₆₋₁₀ arvlene, heteroarylene, heterocyclylene, C₁₋₆ alkylene-O—, C₂₋₆ alkenylene-O—, C₂₋₆ alkynylene-O—, C₁₋₆ alkylene-C(O)O, C₁₋₆ alkylene-NH, C₁₋₆ alkylene-NR^{a2}, C₁₋₆ alkylene-C(O)NH, C₁₋₆ alkylene-C(O)NR^{a2}, C(O), C(O)O, C(O)NH, C(O)NR^{a2}, P(O)(OH), P(O)(NR^{a2}), P(O)(SH), P(S)(NR^{a2}), P(R^{a2}) P(NR^{a2}), S, S(O)₂, S(O), SR^{a2}, and S(NR^{a2}); each R^{a2} is independently selected from C(O)R^{a3}, C(O)OR^{a3}, C(O)NHR^{a3}, C(O)N(C₁₋₄alkyl)^{Ra3}, SR^{a3}, S(O)₂R^{a3}, S(O)R^{a3}, C₁₋₆ alkyl, C₂₋₆ alkenyl, C₂₋₆ alkynyl, C₃₋₁₀ cycloalkyl, C₆₋₁₀ aryl, heteroaryl, and heterocyclyl; each Rₐ³ is independently hydrogen, OH, C₁₋₆ alkyl, C₁₋₆ haloalkyl. C₃₋₁₀ cycloalkyl, C₆₋₁₀ aryl, heteroaryl, or heterocyclyl;
and each RU is a repeating unit independently selected from C₁₋₆ alkylene, C₁₋₆ haloalkylene, C₁₋₆ heteroalkylene, C₂₋₆ alkenylene, C₂₋₆ alkynylene, C₃₋₁₀ cycloalkylene, C₆₋₁₀ arylene, heteroarylene, heterocyclylene, C₁₋₆ alkylene-O—, C₂₋₆ alkenylene-O—, C₂₋₆ alkynylene-O—, C₁₋₆ alkylene-C(O)O, C₁₋₆ alkylene-NH—, C₁₋₆ alkylene-NR^{a2}—, C₁₋₆ alkylene-C(O)NH, C₁₋₆ alkylene-C(O)NR^{a2}, a nucleotide, a saccharide, and an amino acid; and each RU is optionally substituted one with one, two, three, four, or five groups independently selected from halo (for example, fluoro), cyano, azido, OH, OR^{a2}, NO₂, NH₂, NHR^{a2}, N(R^{a2})₂, C₁₋₆ alkyl, C₁₋₆ haloalkyl, C₂₋₆ alkenyl, C₂₋₆ alkynyl, C₃₋₁₀ cycloalkyl, C₆₋₁₀ aryl, heteroaryl, heterocyclyl, C₁₋₆ alkylene-OR^{a2}, C₂₋₆ alkenylene-OR^{a2}, C₂₋₆ alkynylene-OR^{a2}, C₁₋₆ alkylene-NH₂, C₁₋₆ alkylene-NHR^{a2}, C₁₋₆ alkylene-N(R^{a2})₂, C(O)R^{a3}, C(O)OR^{a3}, C(O)NHR^{a3}. C(O)N(C₁₋₄ alkyl)R^{a3}, SR^{a3}, S(O)₂R^{a3}, S(O)R^{a3}, NHC(O)R^{a3}, N(C₁₋₄ alkyl)C(O)R^{a3}, NHS(O)R^{a3}, N(C₁₋₄alkyl)S(O)R^{a3}, NHS(O)₂R^{a3}, and N(C₁₋₄alkyl)S(O)₂R^{a3}; each R^{a2} is independently selected from C(O)R^{a3}, C(O)OR^{a3}, C(O)NHR^{a3}, C(O)N(C₁₋₄alkyl)R^{a3}, SR^{a3}, S(O)₂R^{a3}, S(O)R^{a3}, C₁₋₆ alkyl, C₂₋₆ a lkenyl, C₂₋₆ alkynyl, C₃₋₁₀ cycloalkyl, C₆₋₁₀ aryl, heteroaryl, and heterocyclyl; each R^{a3} is independently hydrogen, OH, C₁₋₆ alkyl, C₁₋₆ haloalkyl, C₃₋₁₀ cycloalkyl, C₆₋₁₀ aryl, heteroaryl, or heterocyclyl; or RU is a hydrophilic moiety; and
wherein q1 is 0-30 and q2 is 1-20.

In certain embodiments, an amino acid has a structure

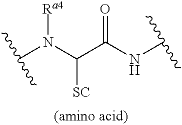

wherein SC is an amino acid side chain, optionally a side chain found in a natural amino acid, and R^{a4} is H or R^{a2} as defined with respected to Formula L. In certain embodiments, SC is H or CH₂OH. In certain embodiments, R^{a2} is methyl.

In certain embodiments, a saccharide has a structure

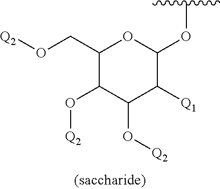

wherein Q¹ is OH, NH₂, or NHAc and wherein two of Q² are H and one of Q² attaches to the remainder of the linker.

In certain embodiments, each RM1 and RM2 is independently selected from phosphate, diphosphate, triphosphate, phosphorothioate, phosphorodithioate, phosphorothiolate; phosphoramidates, alkylphosphonates, CH₂—C(O)NH, O, NH, triazole, amide, carbonate, carbamate, urea. N-hydroxysuccinimide, oxime, hydrazone, disulfide, heteroaryl, heterocyclyl,

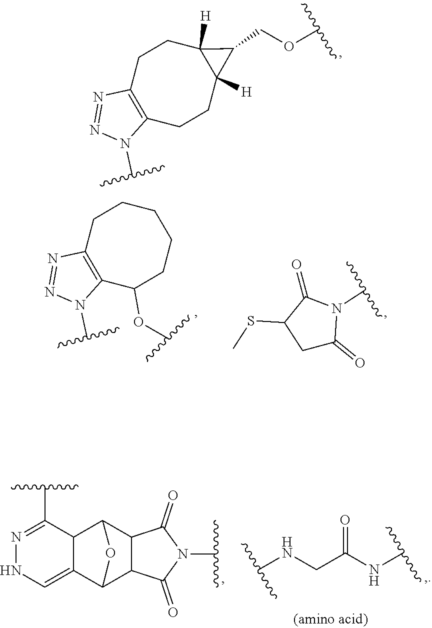

In certain embodiments, each RM1 and RM2 is independently selected from P(O)(OH), O, CH₂—C(O)NH, C(O)NH, or NH, and RU is ethylene glycol or CH₂ optionally substituted with C(O)OH.

In certain embodiments, each RU is a hydrophilic moiety independently selected from O

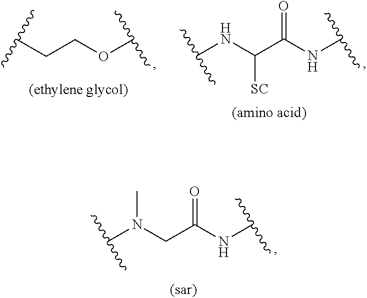

and CH₂, wherein SC is an amino acid side chain, optionally a side chain found in a natural amino acid.

In certain embodiments, each RM1 and RM2 is independently selected from amino acid, C(O)NH, and heteroaryl, and RU is ethylene glycol or optionally substituted CH₂.

In certain embodiments, each RU is ethylene glycol or sar. In certain embodiments, each RU is ethylene glycol and q1 is 2-6. In certain embodiments, each RU is sar and q1 is 2-6. In certain embodiments, each RU is CH₂ and q1 is 1-6.

In certain embodiments, RU is an amino acid, and SC is H or CH₂OH. In certain embodiments, RU comprises glycine and serine. In certain embodiments, (RU)_{q1} is (G₃S)ᵣ₁ or (G₄S)ᵣ₂, wherein G is glycine and S is serine, and r1 and r2 are 1-5. In certain embodiments, (RU)_{q1} is GGGS. In certain embodiments, each RU is an independently selected amino acid and (RU)_{g1} is an amino acid sequence disclosed in U.S. Pat. No. 7,612,181. In certain embodiments, (RU)_{q1} is ASTKGP or TVAAPSVFIFPP. In certain embodiments. (RU)_{q1} is EPKSCDG₄S, EPKSCD(G₄S)₂, or EPKSCD(G₄S)₃. In certain embodiments, (RU)_{q1} is Val-Cit, Gly-Val-Cit, or Gly-Gly-Gly.

### B. Certain Terminal Groups

As used herein, “terminal group” means a chemical group or group of atoms that is covalently linked to a terminus of an oligonucleotide. Examples of a terminal group include, but are not limited to a capping group, a phosphate moiety, a protecting group, a modified or unmodified nucleoside, and two or more nucleosides that are independently modified or unmodified, wherein one or more groups is attached to either or both ends of an oligonucleotide. In certain embodiments, one or more terminal groups is attached to either or both ends of an oligonucleotide. In certain embodiments, one or more terminal groups is attached at the 3′ and/or 5′-end of the oligonucleotide. In certain embodiments, one or more terminal groups is attached at the 3′-end of the oligonucleotide. In certain embodiments, one or more terminal groups is attached at the 5′-end of the oligonucleotide. In certain embodiments, one or more terminal groups is attached at the 3′-end of the oligonucleotide and one or more terminal groups is attached at the 5′-end of the oligonucleotide. In certain embodiments, a terminal group is attached at the 3′ and/or 5′-end of the oligonucleotide. In certain embodiments, a terminal group is attached at the 3′-end of the oligonucleotide. In certain embodiments, a terminal group is attached near the 3′-end of oligonucleotide. In certain embodiments, a terminal group is attached at the 5′-end of the oligonucleotide. In certain embodiments, a terminal group is attached at the 3′-end of the oligonucleotide and a terminal group is attached at the 5′-end of the oligonucleotide.

In certain embodiments, an oligomeric compound comprises one or more terminal groups. In certain embodiments, an oligomeric compound comprises a terminal group comprising a stabilized 5′-phosphate. Stabilized 5′-phosphates include, but are not limited to 5′-vinylphosphonate, 5′-methylphosphonate, and 5′-cyclopropylphosphonate. In certain embodiments, a terminal group comprises one or more abasic sugar moieties. In certain embodiments, a terminal group comprises one or more inverted sugar moieties and/or inverted nucleosides. In certain embodiments, a terminal group comprises one or more 2′-linked nucleosides or sugar moieties. In certain embodiments, the 2′-linked terminal group is an abasic sugar moiety. In certain embodiments, an antisense oligonucleotide comprises a vinylphosphonate. In certain particular embodiments, the antisense oligonucleotide comprises an E-vinyl phosphonate moiety at its 5′-terminus (5′-vP or VP).

### Compositions and Methods for Formulating Pharmaceutical Compositions

In certain embodiments, an oligomeric compound provided herein is a formulated as a pharmaceutical composition. The pharmaceutical composition may contain one or more excipients to facilitate systemic delivery of the oligomeric compound, for example, by infusion or subcutaneous injection. Certain embodiments provide pharmaceutical compositions comprising one or more oligomeric compounds (including oligomeric compounds that are antisense agents or portions thereof) or a salt thereof. In certain such embodiments, the pharmaceutical composition comprises a suitable pharmaceutically acceptable diluent or carrier. In certain embodiments, a pharmaceutical composition comprises a sterile saline solution and one or more oligomeric compound. In certain embodiments, such pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

In certain embodiments, pharmaceutical compositions disclosed herein are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

Under certain conditions, certain oligomeric compounds disclosed herein act as acids. Although such compounds may be drawn or described in protonated (free acid) form, in ionized (anion) form, or ionized and in association with a cation (salt) form, aqueous solutions of such compounds exist in equilibrium among such forms. For example, a phosphate linkage of an oligonucleotide in aqueous solution exists in equilibrium among free acid, anion, and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or salts thereof” expressly includes all such forms that may be fully or partially protonated/de-protonated/in association with a cation. In certain instances, one or more specific cation is identified. Thus provided is a pharmaceutically acceptable salt of an oligomeric compound described herein wherein the salt is sodium or potassium.

### Certain Mechanisms

In certain embodiments, oligomeric compounds (including oligomeric compounds that are antisense agents or portions thereof) described herein comprise or consist of modified oligonucleotides. In certain such embodiments, the oligomeric compounds described herein are capable of hybridizing to a target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Nucleosides that are sufficiently “DNA-like” to elicit RNase H activity are referred to as DNA mimics herein. Further, in certain embodiments, one or more non-DNA-like nucleoside in in the RNA:DNA duplex is tolerated.

In certain antisense activities, hybridization of an antisense agent, oligomeric compound, or modified oligonucleotide described herein to a target nucleic acid results in modulation of the splicing of a target pre-mRNA. For example, in certain embodiments, hybridization of a compound described herein will increase exclusion of an exon. For example, in certain embodiments, hybridization of a compound described herein will increase inclusion of an exon.

In certain antisense activities, antisense agents described herein or a portion of the antisense agent is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi agents. RNAi agents may be double-stranded (siRNA) or single-stranded (ssRNA).

In certain antisense activities, antisense agents, oligomeric compounds, or modified oligonucleotides described herein result in a CRISPR system cleaving a target DNA. In certain antisense activities, compounds described herein result in a CRISPR system editing a target DNA.

In certain antisense activities, hybridization of an antisense agent, oligomeric compound, or modified oligonucleotide described herein to a target nucleic acid results in disruption of secondary structural elements, such as stem-loops and hairpins. For example, in certain embodiments, hybridization of a compound described herein to a stem-loop that is part of a translation suppression element leads to an increase in protein expression.

In certain antisense activities, hybridization of an antisense agent, oligomeric compound, or modified oligonucleotide described herein to a target nucleic acid leads to no-go decay mediated mRNA degradation.

In certain antisense activities, hybridization of an antisense agent, oligomeric compound, or modified oligonucleotide described herein to a target nucleic acid leads to activation of nonsense-mediated decay mRNA degradation.

In certain embodiments, antisense agents, oligomeric compounds, or modified oligonucleotides described herein are artificial mRNA compounds, the nucleobase sequence of which encodes for a protein.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

Target Nucleic Acids. Target Regions and Nucleotide Sequences

In certain embodiments, antisense agents, oligomeric compounds, or modified oligonucleotides described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain embodiments, a pre-mRNA and corresponding mRNA are both target nucleic acids of a single compound. In certain such embodiments, the target region is entirely within an intron of a target pre-mRNA. In certain embodiments, the target region spans an intron/exon junction. In certain embodiments, the target region is at least 50% within an intron. In certain embodiments, the target nucleic acid is a microRNA. In certain embodiments, the target region is in the 5′ UTR of a gene. In certain embodiments, the target region is within a translation suppression element region of a target nucleic acid.

### Certain Isomers and Isotopes

Certain compounds described herein (e.g., antisense agents, oligomeric compounds, and modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations are enriched (e.g., to at least about 50% ee) for the indicated stereochemistry. Compounds provided herein that are drawn or described with undefined stereochemistry include racemic, stereorandom, and optically enriched forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ²C, ¹⁵N in place of ¹⁴N, ¹⁷O or ¹⁸O in place of ¹⁶O, and ³³S, ³⁴s, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### Synthesis

Various methods for synthesis of isomeric furanose nucleosides and their phosphoramidites are known in the art. Relevant synthesis are described in, e.g., WO 2021/030763 and WO 2020/072991.

### EXAMPLES

The following examples are intended to illustrate certain aspects of the invention and are not intended to limit the invention in any way.

### Example 1: Design of Modified RNAi Compounds Targeted to Mouse TTR

RNAi compounds comprising antisense oligonucleotides complementary to a mouse TTR nucleic acid, and sense RNAi oligonucleotides complementary to the antisense RNAi oligonucleotides were designed as follows.

### Design of Antisense Oligonucleotides

The antisense RNAi oligonucleotides described in the table below are 23 nucleosides in length, have the sequence TUAUAGAGCAAGAACACUGUUUU (SEQ ID NO: 2), and are complementary to mouse TTR (GenBank Accession No. NM_013697.5 (SEQ ID NO: 1)) from nucleoside start site 691 to nucleoside 713. Each antisense oligonucleotide in the table below has a sugar motif as designated in the column labeled “Antisense Strand Sugar Motif (5′ to 3′)”, wherein each ‘e’ represents a stereo-standard 2′-MOE sugar moiety, each ‘y’ represents a stereo-standard 2′-OMe sugar moiety, each ‘f’ represents a stereo-standard 2′-F sugar moiety, each ‘[bDdx]’ represents a 2′-β-D-deoxyxylose sugar moiety, each ‘[aLdr]’ represents a 2′-α-L-deoxyribose sugar moiety, and each “[Sgna]” represents a (S)-GNA moiety; and an internucleoside linkage motif as designated in the column labeled “Sense Strand Internucleoside Linkage (5′ to 3′)”, wherein each ‘o’ represents a phosphodiester internucleoside linkage, and each ‘s’ represents a phosphorothioate internucleoside linkage. Each antisense oligonucleotide has a vinyl phosphonate moiety on the 5′-end.

Compound No. 1708610 was previously disclosed in U.S. Application 63/387,478.

TABLE 1
Design of antisense RNAi oligonucleotides targeted to mouse TTR

| | | | Antisense | |
|---|---|---|---|---|
| | | Antisense Strand | Internucleoside | SEQ |
| Compound | Antisense Sequence | Sugar Motif | Linkage Motif | ID |
| No. | (5′ to 3′) | (5′ to 3′) | (5′ to 3′) | NO. |
| 1708610 | TUAUAGAGCAAGA | efyyyfyyyyyyyfyfyy | ssooooooooo | 2 |
| | ACACUGUUUU | yyyyy | oooooooooss | |
| 1743128 | TUAUAGAGCAAGA | efyyyf[bDdx]yyyyyy | ssooooooooo | 2 |
| | ACACUGUUUU | fyfyyyyyyy | oooooooooss | |
| 1757519 | TUAUAGAGCAAGA | efyyy[bDdx]yyyyyyy | ssooooooooo | 2 |
| | ACACUGUUUU | fyfyyyyyyy | oooooooooss | |
| 1757520 | TUAUAGAGCAAGA | efyyyf[aLdr]yyyyyy | ssooooooooo | 2 |
| | ACACUGUUUU | fyfyyyyyyy | oooooooooss | |
| 1757521 | TUAUAGAGCAAGA | efyyy [aLdr]yyyyyy | ssooooooooo | 2 |
| | ACACUGUUUU | yfyfyyyyyyy | oooooooooss | |
| 1719186 | TUAUAGAGCAAGA | efyyyf[Sgna]yyyyyy | ssooooooooo | 2 |
| | ACACUGUUUU | fyfyyyyyyy | oooooooooss | |

### Design of Sense Oligonucleotides

The sense RNAi oligonucleotide described in the table below is 21 nucleosides in length and is complementary to the first 21 nucleosides of the antisense oligonucleotide (from 5′ to 3′) wherein the last two 3′-nucleosides of the antisense oligonucleotides are not paired with the sense oligonucleotide (are overhanging nucleosides). The sense oligonucleotide has a sugar motif as designated in the column labeled “Sense Strand Sugar Motif (5′ to 3′)”, wherein each ‘y’ represents a stereo-standard 2′-OMe sugar moiety, and each ‘f’ represents a stereo-standard 2′-F sugar moiety; and an internucleoside linkage motif as designated in the column labeled “Sense Strand Internucleoside Linkage (5′ to 3′)”, wherein each ‘o’ represents a phosphodiester internucleoside linkage, and each ‘s’ represents a phosphorothioate internucleoside linkage. The sense oligonucleotide in the table below is conjugated to a HPPO-GalNAc moiety at the 3′-OH of the oligonucleotide, as shown below:

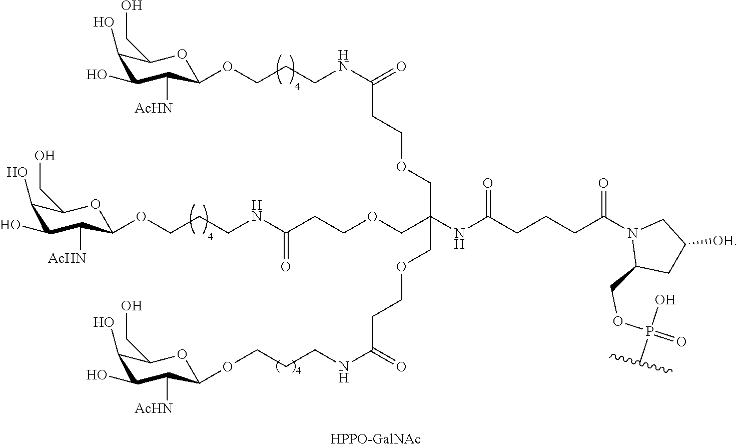

Compound No. 1708605 was previously disclosed in U.S. Application 63/387,478.

TABLE 2
Design of sense RNAi oligonucleotide
with a 3′ GalNAc conjugate

| | | | Inter- | |
|---|---|---|---|---|
| Com- | | Sugar | nucleoside | SEQ |
| pound | Sequence | Motif | linkages | ID |
| No. | (5′ to 3′) | (5′ to 3′) | (5′ to 3′) | NO. |
| 1708605 | AACAGUGUUCU | yyyyyyfyfff | ssooooooooo | 3 |
| | UGCUCUAUAA | yyyyyyyyyy | ooooooooooo | |

### Design of Rnai Duplexes

RNAi duplex compounds prepared with antisense oligonucleotide compound numbers and corresponding sense oligonucleotide compound numbers are listed in Table 3. Compound No. 1708616 was previously disclosed in U.S. Application 63/387,478.

TABLE 3
Design of oligomeric duplexes targeted to mouse TTR

| | Antisense | |
|---|---|---|
| Duplex Compound No. | Compound No. | Sense Compound No. |
| 1708616 | 1708610 | 1708605 |
| 1757524 | 1743128 | 1708605 |
| 1757527 | 1757520 | 1708605 |
| 1757530 | 1757519 | 1708605 |
| 1757533 | 1757521 | 1708605 |
| 1722757 | 1719186 | 1708605 |

### Example 2: Effect of Modified RNAi Compounds Targeted to TTR on On-Target Vs Off-Target Activity

The psiCHECK™ reporter vector (Promega) and Dual-Glo® Luciferase Assay System (Promega) were used to compare the effect of RNAi compounds described herein above on on-target vs off-target activity.

The on-target reporter vector contained a single fully complementary site to the antisense strand of TTR siRNA consisting of the sequence (from 5′ to 3′) AAAACAGTGTTCTTGCTCTATAA (SEQ ID NO: 4) inserted into the 3′-untranslated region of the Renilla luciferase cassette. The off-target reporter vector contained four seed-complementary sites consisting of the sequence (from 5′ to 3′) GCTCTATAA separated by a 19-nucleotide spacer sequence (from 5′ to 3′) TAATATTACATAAATAAAA (SEQ ID NO: 5) inserted into the 3′ untranslated region of the Renilla cassette.

Cos7 cells (ATCC, Manassas, VA) were grown to near confluence before trypsinization siRNA duplexes (described herein above) and psiCHCECK2 plasmids (Promega) that contain either the on-target or off-target sequences (described herein above) were co-transfected by adding siRNA duplexes at concentrations indicated in the table below together with 5 μl (10 ng) of psiCHECK2 plasmid per well along with 5 μl of Opti-MEM that had been premixed with Lipofectamine 2000 (2 μg/ml) and then incubated at room temperature for 15 min. The mixture was then added to the cells which had been cultured overnight in 100 μl complete media 48 hours post transfection, levels of Firefly luciferase (transfection control) and Renilla luciferase (fused either to on-target or off-target sequence) were measured per manufacturer protocol, siRNA activity was determined by normalizing the Renilla signal to the Firefly (control) signal within each well. The magnitude of siRNA activity was then assessed relative to cells that had been transfected with psiCHECK2 plasmid in the absence of siRNA (% control). TC₅₀ values were calculated using Prism software under a 4-parameter non-linear dose response function and are presented in the table below. “N.D.” indicates the value was not determined.

TABLE 4
Effect of RNAi duplexes on-target vs off-target

| | | On-Target | | | Off-Target | |
|---|---|---|---|---|---|---|
| Compound | Concentration | On-target | IC₅₀ | Concentration | Off-target | IC₅₀ |
| No. | (pM) | (% control) | (pM) | (pM) | (% control) | (pM) |
| 1708616 | 2000 | 6 | 0.67 | 50000 | 14 | 15 |
| | 286 | 7 | | 10000 | 20 | |
| | 40.8 | 10 | | 2000 | 20 | |
| | 5.83 | 24 | | 400 | 29 | |
| | 0.83 | 53 | | 80 | 38 | |
| | 0.12 | 73 | | 16 | 49 | |
| | 0.017 | 84 | | 3.2 | 68 | |
| | 0.0024 | 83 | | 0.64 | 67 | |
| | 0.00035 | 90 | | 0.128 | 67 | |
| | 0.00005 | 102 | | 0.0256 | 89 | |
| | 0.000007 | 109 | | 0.00512 | 99 | |
| | 0.000001 | 110 | | 0.00102 | 101 | |
| 1757524 | 2000 | 7 | 0.79 | 50000 | 33 | 2688 |
| | 286 | 8 | | 10000 | 45 | |
| | 40.8 | 11 | | 2000 | 48 | |
| | 5.83 | 28 | | 400 | 59 | |
| | 0.83 | 52 | | 80 | 74 | |
| | 0.12 | 78 | | 16 | 91 | |
| | 0.017 | 79 | | 3.2 | 92 | |
| | 0.0024 | 90 | | 0.64 | 93 | |
| | 0.00035 | 95 | | 0.128 | 97 | |
| | 0.00005 | 99 | | 0.0256 | 103 | |
| | 0.000007 | 106 | | 0.00512 | 100 | |
| | 0.000001 | 107 | | 0.00102 | 103 | |
| 1757527 | 2000 | 7 | 0.48 | 50000 | 57 | N.D. |
| | 286 | 8 | | 10000 | 87 | |
| | 40.8 | 10 | | 2000 | 85 | |
| | 5.83 | 19 | | 400 | 90 | |
| | 0.83 | 48 | | 80 | 92 | |
| | 0.12 | 73 | | 16 | 93 | |
| | 0.017 | 78 | | 3.2 | 99 | |
| | 0.0024 | 85 | | 0.64 | 101 | |
| | 0.00035 | 89 | | 0.128 | 106 | |
| | 0.00005 | 103 | | 0.0256 | 102 | |
| | 0.000007 | 107 | | 0.00512 | 92 | |
| | 0.000001 | 122 | | 0.00102 | 96 | |
| 1757530 | 2000 | 5 | 0.15 | 50000 | 35 | 10080 |
| | 286 | 6 | | 10000 | 57 | |
| | 40.8 | 7 | | 2000 | 61 | |
| | 5.83 | 21 | | 400 | 72 | |
| | 0.83 | 41 | | 80 | 91 | |
| | 0.12 | 63 | | 16 | 91 | |
| | 0.017 | 67 | | 3.2 | 94 | |
| | 0.0024 | 72 | | 0.64 | 103 | |
| | 0.00035 | 82 | | 0.128 | 108 | |
| | 0.00005 | 98 | | 0.0256 | 99 | |
| | 0.000007 | 98 | | 0.00512 | 93 | |
| | 0.000001 | 102 | | 0.00102 | 98 | |
| 1757533 | 2000 | 9 | 0.41 | 50000 | 38 | 8488 |
| | 286 | 9 | | 10000 | 53 | |
| | 40.8 | 10 | | 2000 | 60 | |
| | 5.83 | 21 | | 400 | 70 | |
| | 0.83 | 50 | | 80 | 77 | |
| | 0.12 | 70 | | 16 | 88 | |
| | 0.017 | 74 | | 3.2 | 94 | |
| | 0.0024 | 79 | | 0.64 | 96 | |
| | 0.00035 | 90 | | 0.128 | 98 | |
| | 0.00005 | 103 | | 0.0256 | 100 | |
| | 0.000007 | 107 | | 0.00512 | 101 | |
| | 0.000001 | 104 | | 0.00102 | 111 | |

TABLE 5
Effect of RNAi duplexes on-target vs off-target

| Duplex | | On-Target | | Off-Target | |
|---|---|---|---|---|---|
| Compound | Concentration | On-target | IC₅₀ | Of-target | IC₅₀ |
| No. | (pM) | (% control) | (pM) | (% control) | (pM) |
| 1708616 | 50000 | N.D. | 0.05 | 14 | 31.2 |
| | 10000 | N.D. | | 20 | |
| | 2000 | 33 | | 19 | |
| | 400 | 45 | | 24 | |
| | 80 | 45 | | 39 | |
| | 16 | 38 | | 64 | |
| | 3.2 | 50 | | 78 | |
| | 0.64 | 65 | | 72 | |
| | 0.128 | 72 | | 68 | |
| | 0.026 | 79 | | 72 | |
| | 0.005 | 86 | | 100 | |
| | 0.001 | 92 | | 100 | |
| | 0.0002 | 104 | | N.D. | |
| | 0.00004 | 96 | | N.D. | |
| 1722757 | 50000 | N.D. | 0.17 | 40 | N.D. |
| | 10000 | N.D. | | 81 | |
| | 2000 | 19 | | 90 | |
| | 400 | 22 | | 70 | |
| | 80 | 32 | | 93 | |
| | 16 | 32 | | 96 | |
| | 3.2 | 46 | | 97 | |
| | 0.64 | 67 | | 93 | |
| | 0.128 | 61 | | 101 | |
| | 0.026 | 78 | | 88 | |
| | 0.005 | 85 | | 97 | |
| | 0.001 | 80 | | 103 | |
| | 0.0002 | 102 | | N.D. | |
| | 0.00004 | 98 | | N.D. | |

### Example 3: Design of Modified RNAi Compounds Targeted to Mouse ACTN1

RNAi compounds comprising antisense oligonucleotides complementary to a mouse ACTN1 nucleic acid, and sense RNAi oligonucleotides complementary to the antisense RNAi oligonucleotides were designed as follows.

### Design of Antisense Oligonucleotides

The antisense RNAi oligonucleotides described in the table below are 23 nucleosides in length, and are complementary to mouse ACTN1 (GenBank Accession No. NM_134156.2 (SEQ ID NO: 21)), wherein some antisense RNAi oligonucleotides are 100% complementary, and some antisense RNAi oligonucleotides have a single mismatched thymine at the 5′-end, indicated in bold. Each antisense oligonucleotide in the table below has a sugar motif as designated in the column labeled “Antisense Strand Sugar Motif (5′ to 3′)”, wherein each “e” represents a stereo-standard 2′-MOE sugar moiety, each “y” represents a stereo-standard 2′-OMe sugar moiety, each “f” represents a stereo-standard 2′-F sugar moiety, each “[bDdx]” represents a 2′-β-D-deoxyxylose sugar moiety, each “[aLdr]” represents a 2′-α-L-deoxyribose sugar moiety, each “[m2bDx]” represents a 2′-OMe-β-D-deoxyxylose sugar moiety, each “[f2bDx]” represents a 2′-fluoro-β-D-xylose sugar moiety, and each “[Sgna]” represents a (S)-GNA moiety. Each antisense oligonucleotide in the table below has an internucleoside linkage motif as designated in the column labeled “Antisense Strand Internucleoside Linkage (5′ to 3′)”, wherein each “o” represents a phosphodiester internucleoside linkage, and each “s” represents a phosphorothioate internucleoside linkage. Each antisense oligonucleotide has a vinyl phosphonate moiety on the 5′-end. Each cytosine nucleobase represented with bolded, italicized, and underlined text is a 5-methylcytosine.

TABLE 6
Design of antisense RNAi oligonucleotides targeted to mouse ACTN1

| | Antisense Strand | Antisense Strand | Internucleoside | SEQ |
|---|---|---|---|---|
| Compound | Sequence | Sugar Motif | linkages | ID |
| No. | (5′ to 3′) | (5′ to 3′) | (5′ to 3′) | NO. |
| 1744535 | TAAGUGAGCUAGC | efyyyfyyyyyyyfyfyyyyyyy | ssooooooooooooooooooss | 6 |
| | AAACACACAU | | | |
| 1788292 | TAAGUGAGCUAGC | efyyyf[Sgna]yyyyyyfyfyyyyyyy | ssooooooooooooooooooss | 6 |
| | AAACACACAU | | | |
| 1788293 | TAAGUGAGCUAGC | efyyyf[aLdr]yyyyyyfyfyyyyyyy | ssooooooooooooooooooss | 6 |
| | AAACACACAU | | | |
| 1788294 | TAAGUGAGCUAGC | efyyy[bDdx]yyyyyyyfyfyyyyyyy | ssooooooooooooooooooss | 6 |
| | AAACACACAU | | | |
| 1825797 | CAAGUGAGCUAGC | efyyy[aLdr]yyyyyyyfyfyyyyyyy | ssooooooooooooooooooss | 7 |
| | AAACACACAU | | | |
| 1825800 | CAAGUGAGCUAGC | efyyyf[bDdx]yyyyyyfyfyyyyyyy | ssooooooooooooooooooss | 7 |
| | AAACACACAU | | | |
| 1785519 | TAAUUUUGUAAAC | efyyyfyyyyyyyfyfyyyyyyy | ssooooooooooooooooooss | 8 |
| | UGUCACUUUG | | | |
| 1853609 | TAAUUTUGUAAAC | efyyy[m2bDx]yyyyyyyfyfyyyyyyy | ssooooooooooooooooooss | 9 |
| | UGUCACUUUG | | | |
| 1859765 | TAAUUUUGUAAAC | efyyy[f2bDx]yyyyyyyfyfyyyyyyy | ssooooooooooooooooooss | 8 |
| | UGUCACUUUG | | | |
| 1853611 | TAAUUUUGUAAAC | efyyy[bDdx]yyyyyyyfyfyyyyyyy | ssooooooooooooooooooss | 8 |
| | UGUCACUUUG | | | |

### Design of Sense Oligonucleotides

The sense RNAi oligonucleotide described in the table below is 21 nucleosides in length and is complementary to the first 21 nucleosides of the antisense oligonucleotide (from 5′ to 3′) wherein the last two 3′-nucleosides of the antisense oligonucleotides are not paired with the sense oligonucleotide (are overhanging nucleosides). The sense oligonucleotide has a sugar motif as designated in the column labeled “Sense Strand Sugar Motif (5′ to 3′)”, wherein each “y” represents a stereo-standard 2′-OMe sugar moiety, and each “f” represents a stereo-standard 2′-F sugar moiety; and an internucleoside linkage motif as designated in the column labeled “Sense Strand Interucleoside Linkage (5′ to 3′)”, wherein each “o” represents a phosphodiester internucleoside linkage, and each “s” represents a phosphorothioate internucleoside linkage. The sense oligonucleotide in the table below is conjugated to a HPPO-GalNAc moiety at the 3′-OH of the oligonucleotide, shown herein above.

TABLE 7
Design of sense RNAi oligonucleotide
with a 3′ GalNAc conjugate

| | | | Sense | |
|---|---|---|---|---|
| | | | Strand | |
| | | | Inter- | |
| | | | nucleo- | |
| Com- | Sense Strand | Sense Strand | side | SEQ |
| pound | Sequence | Sugar Motif | linkages | ID |
| No. | (5′ to 3′) | (5′ to 3′) | (5′ to 3′) | NO. |
| 1744544 | GUGUGUUUGCU | yyyyyyfyfff | ssoooooooo | 10 |
| | AGCUCACUUA | yyyyyyyyyy | ooooooooss | |
| 1825795 | GUGUGUUUGCU | yyyyyyfyfff | ssoooooooo | 11 |
| | AGCUCACUUG | yyyyyyyyyy | ooooooooss | |
| 1785518 | AAGUGACAGUU | yyyyyyfyfff | ssoooooooo | 12 |
| | UACAAAAUUA | yyyyyyyyyy | oooooooooo | |

### Design of RNAi Duplexes

RNAi duplex compounds prepared with antisense oligonucleotide compound numbers and corresponding sense oligonucleotide compound numbers are listed in the table below.

TABLE 8
Design of oligomeric duplexes targeted to mouse ACTN1

| Duplex Compound | Antisense | Sense Compound |
|---|---|---|
| No. | Compound No. | No. |
| 1744547 | 1744535 | 1744544 |
| 1788303 | 1788292 | 1744544 |
| 1788306 | 1788293 | 1744544 |
| 1788309 | 1788294 | 1744544 |
| 1785522 | 1785519 | 1785518 |
| 1825799 | 1825797 | 1825795 |
| 1825802 | 1825800 | 1825795 |
| 1853612 | 1853609 | 1785518 |
| 1859766 | 1859765 | 1785518 |
| 1853614 | 1853611 | 1785518 |

### Example 4: Selectivity of Oligomeric Duplexes that Target an ACTN1 RNA in A431 Cells

Off-target effects of modified oligonucleotides complimentary to ACTN1 RNA selected from the examples above were tested in A431 cells. A431 cells were plated at a density of 10,000 cells per well and treated with oligomeric duplex compounds at concentrations of 5000 nM, 1000 nM, 200 nM, 40 nM, 8 nM. 1.6 nM, 0.32 nM, 0.064 nM. 0.0128 nM, and 0.002 nM using RNAimax. After a treatment period of approximately 96 hours, total RNA was isolated from cells, and subjected to DGE (Digital Gene Expression) analysis for 3′ end transcriptome profiling using the QuantSeq 3′ mRNA-Seq Library Prep Kit FWD for Illumina on the Illumina sequencing platform. DGE analysis resulted in the generation of greater than 2.5 million unique, mapped reads, with expression data for greater than 11050 unigenes. Knockdown was measured as a concentration response experiment and IC₅₀ values were determined for concentration responsive genes demonstrating knockdown. Reduction of on-target (ACTN1) mRNA was compared to the IC₅₀ of all concentration responsive genes that demonstrated knockdown. The number of differentially expressed off-target genes detected with each oligomeric duplex treatment were identified as critical responders when the off-targets showed at least 50% reduction, and showed not more than 10-fold lower potency than the on-target. The number of differentially expressed off-target genes detected with each oligomeric duplex treatment were identified as responders when the off-targets showed at least 50% reduction, and had a potency 10× or lower that of the on-target.

TABLE 9
Number of critical responders and responders
detected with oligomeric duplex treatment

| | Number of | |
|---|---|---|
| Compound | Critical | Number of |
| No. | Responders | Responders |
| 1744547 | 10 | 69 |
| 1788303 | 0 | 9 |
| 1788306 | 3 | 8 |
| 1788309 | 0 | 0 |

### Example 5: Selectivity of Oligomeric Duplexes that Target a TTR RNA In Vivo

Wild type C57BL/6 mice (Jackson Laboratory) were used to determine the selectivity of oligomeric duplexes described herein above.

### Treatment

Groups of 4 male C57BL/6 mice each received a single subcutaneous injection of RNAi duplex compound at doses of 0.1 mg/kg, 0.3 mg/kg. 1 mg/kg, 3 mg/kg. 10 mg/kg. 30 mg/kg, and 100 mg/kg. One group of 4 mice received a single subcutaneous injection of PBS and served as a control group to which RNAi duplex-treated groups were compared. 7 days post treatment, the mice were euthanized.

### RNA Analysis

7 days post treatment, mice were sacrificed and RNA was extracted from liver tissue for quantitative real-time RTPCR analysis of RNA expression of TTR using mouse primer probe set mTTR_1 (forward sequence CGTACTGGAAGACACTTGGCATT, designated herein as SEQ ID NO: 13; reverse sequence GAGTCGTTGGCTGTGAAAACC, designated herein as SEQ ID NO: 14; probe sequence CCCGTTCCATGAATTCGCGGATG, designated herein as SEQ ID NO: 15). TTR RNA levels were normalized to total RNA content, as measured by RIBOGREEN®. Results are presented as percent mouse TTR RNA relative to the amount of TTR RNA in PBS treated animals, (% control). The half maximal effective dose (ED50) of each oligomeric duplex was calculated using GraphPad Prism 10 software (GraphPad Software, San Diego. CA).

TABLE 10
Reduction of mouse TTR RNA in wild type mice

| Compound | Dose | TTR RNA | ED50 |
|---|---|---|---|
| No. | (mg/kg) | (% control) | (mg/kg) |
| 1708616 | 0.1 | 31 | 0.05 |
| | 0.3 | 12 | |
| | 1 | 2 | |
| | 3 | 1 | |
| | 10 | 0.3 | |
| | 30 | 0.2 | |
| | 100 | 0.3 | |
| 1722757 | 0.1 | 53 | 0.12 |
| | 0.3 | 28 | |
| | 1 | 5 | |
| | 3 | 1 | |
| | 10 | 0.3 | |
| | 30 | 0.5 | |
| | 100 | 0.2 | |
| 1757527 | 0.1 | 48 | 0.10 |
| | 0.3 | 27 | |
| | 1 | 3 | |
| | 3 | 1 | |
| | 10 | 1 | |
| | 30 | 0.4 | |
| | 100 | 0.4 | |

TABLE 11
Reduction of mouse TTR RNA in wild type mice

| Compound | Dose | TTR RNA | ED50 |
|---|---|---|---|
| No. | (mg/kg) | (% control) | (mg/kg) |
| 1757530 | 0.1 | 51 | 0.10 |
| | 0.3 | 22 | |
| | 1 | 7 | |
| | 3 | 1 | |
| | 10 | 0.4 | |
| | 30 | 0.3 | |
| | 100 | 0.3 | |

### DGE Analysis

Liver tissue extracted from the mice was subjected to DGE (Digital Gene Expression) analysis for 3′ end transcriptome profiling using the QuantSeq 3′ mRNA-Seq Library Prep Kit FWD for Illumina on the Illumina sequencing platform.

Digital gene expression analysis resulted in the generation of greater than 2.5 million unique, mapped reads per sample, with expression data for greater than 10,000 unigenes. A total of 17 unique differentially expressed genes with a fold change greater than 2-fold downregulation, a p value less than 0.01 and a q value less than 0.1 were identified upon comparison of the saline and the RNAi duplex treated groups. The seventeen differentially expressed genes includes the on-target gene (TTR) for each sample.

The number of differentially expressed genes detected with each RNAi duplex treatment are listed in the table below.

TABLE 12
Number of differentially expressed genes
detected with RNAi duplex treatment

| | Number of |
|---|---|
| | differentially expressed |
| Compound No. | genes detected |
| 1708616 | 14 |
| 1722757 | 2 |
| 1757527 | 4 |
| 1757530 | 1 |

### Example 6: Effect of Modified RNAi Compounds Targeted to ACTN1 on On-Target Vs Off-Target Activity

The psiCHECK™ reporter vector (Promega) and Dual-Glo® Luciferase Assay System (Promega) were used to compare the effect of RNAi compounds described herein above on on-target vs off-target activity.

The on-target reporter vector contained a single fully complementary site to the antisense strand of ACTN1 siRNA consisting of the sequence (from 5′ to 3′) ATGTGTGTTTGCTAGCTCACTTA (SEQ ID NO: 16) inserted into the 3′-untranslated region of the Renilla luciferase cassette. The off-target reporter vector contained four seed-complementary sites consisting of the sequence (from 5′ to 3′) GCTCACTTA separated by a 19-nucleotide spacer sequence (from 5′ to 3′) TAATATTACAAAAATAAAT (SEQ ID NO: 17) inserted into the 3′ untranslated region of the Renilla cassette.

Cos7 cells (ATCC, Manassas, VA) were grown to near confluence before trypsinization, siRNA duplexes (described herein above) and psiCHECK2 plasmids (Promega) that contain either the on-target or off-target sequences (described herein above) were co-transfected by adding siRNA duplexes at concentrations indicated in the table below together with 5 μl, (10 ng) of psiCHECK2 plasmid per well along with 5 μl of Opti-MEM that had been premixed with Lipofectamine 2000 (2 μg/ml) and then incubated at room temperature for 15 min. The mixture was then added to the cells which had been cultured overnight in 100 μL complete media

48 hours post transfection, levels of Firefly luciferase (transfection control) and Renilla luciferase (fused either to on-target or off-target sequence) were measured per manufacturer protocol, siRNA activity was determined by normalizing the Renilla signal to the Firefly (control) signal within each well. The magnitude of siRNA activity was then assessed relative to cells that had been transfected with psiCHECK2 plasmid in the absence of siRNA (9 control). IC₅₀ values were calculated using Prism software using a 4-parameter non-linear dose response function and are presented in the table below. “N.D.” indicates the value was not determined. Each table represents a separate experiment.

TABLE 13
Effect of RNAi duplexes on-target vs off-target

| | | On-Target | | Off-Target | |
|---|---|---|---|---|---|
| Duplex | | On-target | | Of-target | |
| Compound | Concentration | (% | IC₅₀ | (% | IC₅₀ |
| No. | (pM) | control) | (pM) | control) | (pM) |
| 1744547 | 50000 | N.D. | 2.46 | 14 | 11.5 |
| | 10000 | N.D. | | 15 | |
| | 2000 | 2 | | 20 | |
| | 400 | 2 | | 25 | |
| | 80 | 4 | | 31 | |
| | 16 | 12 | | 44 | |
| | 3.2 | 43 | | 53 | |
| | 0.64 | 79 | | 66 | |
| | 0.128 | 101 | | 87 | |
| | 0.026 | 100 | | 96 | |
| | 0.005 | 105 | | 104 | |
| | 0.001 | 102 | | 128 | |
| | 0.0002 | 101 | | N.D. | |
| | 0.00004 | 99 | | N.D. | |
| 1788303 | 50000 | N.D. | 1.16 | 58 | 79580 |
| | 10000 | N.D. | | 65 | |
| | 2000 | 2 | | 81 | |
| | 400 | 2 | | 84 | |
| | 80 | 2 | | 92 | |
| | 16 | 6 | | 104 | |
| | 3.2 | 22 | | 104 | |
| | 0.64 | 67 | | 102 | |
| | 0.128 | 96 | | 101 | |
| | 0.026 | 96 | | 101 | |
| | 0.005 | 102 | | 108 | |
| | 0.001 | 99 | | 92 | |
| | 0.0002 | 101 | | N.D. | |
| | 0.00004 | 90 | | N.D. | |
| 1788306 | 50000 | N.D. | 0.70 | 36 | 4489 |
| | 10000 | N.D. | | 38 | |
| | 2000 | 2 | | 56 | |
| | 400 | 2 | | 65 | |
| | 80 | 2 | | 86 | |
| | 16 | 5 | | 97 | |
| | 3.2 | 17 | | 97 | |
| | 0.64 | 51 | | 103 | |
| | 0.128 | 88 | | 102 | |
| | 0.026 | 93 | | 103 | |
| | 0.005 | 103 | | 102 | |
| | 0.001 | 101 | | 98 | |
| | 0.0002 | 99 | | N.D. | |
| | 0.00004 | 90 | | N.D. | |
| 1788309 | 50000 | N.D. | 1.19 | 34 | 387 |
| | 10000 | N.D. | | 24 | |
| | 2000 | 3 | | 35 | |
| | 400 | 2 | | 28 | |
| | 80 | 3 | | 63 | |
| | 16 | 9 | | 84 | |
| | 3.2 | 31 | | 97 | |
| | 0.64 | 62 | | 97 | |
| | 0.128 | 90 | | 101 | |
| | 0.026 | 93 | | 95 | |
| | 0.005 | 94 | | 102 | |
| | 0.001 | 94 | | 98 | |
| | 0.0002 | 106 | | N.D. | |
| | 0.00004 | 97 | | N.D. | |

TABLE 14
Effect of RNAi duplexes on-target vs off-target

| | | On-Target | | Off-Target | |
|---|---|---|---|---|---|
| Duplex | | On-target | | Of-target | |
| Compound | Concentration | (% | IC₅₀ | (% | IC₅₀ |
| No. | (pM) | control) | (pM) | control) | (pM) |
| 1744547 | 50000 | N.D. | 2.94 | 9 | 3.16 |
| | 10000 | N.D. | | 10 | |
| | 2000 | 3 | | 13 | |
| | 400 | 3 | | 16 | |
| | 80 | 5 | | 21 | |
| | 16 | 16 | | 30 | |
| | 3.2 | 49 | | 44 | |
| | 0.64 | 81 | | 60 | |
| | 0.128 | 92 | | 84 | |
| | 0.026 | 100 | | 97 | |
| | 0.005 | 101 | | 103 | |
| | 0.001 | 106 | | 127 | |
| | 0.0002 | 96 | | N.D. | |
| | 0.00004 | 104 | | N.D. | |
| 1788303 | 50000 | N.D. | 1.37 | 64 | 143900 |
| | 10000 | N.D. | | 66 | |
| | 2000 | 2 | | 84 | |
| | 400 | 2 | | 95 | |
| | 80 | 3 | | 92 | |
| | 16 | 8 | | 101 | |
| | 3.2 | 31 | | 101 | |
| | 0.64 | 67 | | 92 | |
| | 0.128 | 94 | | 97 | |
| | 0.026 | 90 | | 107 | |
| | 0.005 | 97 | | 104 | |
| | 0.001 | 101 | | 96 | |
| | 0.0002 | 99 | | N.D. | |
| | 0.00004 | 100 | | N.D. | |
| 1825799 | 50000 | N.D. | 2.70 | 18 | 64 |
| | 10000 | N.D. | | 20 | |
| | 2000 | 3 | | 24 | |
| | 400 | 2 | | 26 | |
| | 80 | 5 | | 48 | |
| | 16 | 13 | | 66 | |
| | 3.2 | 51 | | 76 | |
| | 0.64 | 73 | | 81 | |
| | 0.128 | 99 | | 85 | |
| | 0.026 | 100 | | 84 | |
| | 0.005 | 96 | | 100 | |
| | 0.001 | 97 | | 100 | |
| | 0.0002 | 103 | | N.D. | |
| | 0.00004 | 103 | | N.D. | |
| 1825802 | 50000 | N.D. | 1.59 | 18 | 63 |
| | 10000 | N.D. | | 15 | |
| | 2000 | 3 | | 21 | |
| | 400 | 3 | | 25 | |
| | 80 | 4 | | 49 | |
| | 16 | 10 | | 65 | |
| | 3.2 | 36 | | 73 | |
| | 0.64 | 68 | | 85 | |
| | 0.128 | 92 | | 93 | |
| | 0.026 | 96 | | 99 | |
| | 0.005 | 104 | | 97 | |
| | 0.001 | 96 | | 103 | |
| | 0.0002 | 104 | | N.D. | |
| | 0.00004 | 108 | | N.D. | |

TABLE 15
Effect of RNAi duplexes on-target vs off-target

| | | On-Target | | Off-Target | |
|---|---|---|---|---|---|
| Duplex | | On-target | | Of-target | |
| Compound | Concentration | (% | IC₅₀ | (% | IC₅₀ |
| No. | (pM) | control) | (pM) | control) | (pM) |
| 1785522 | 50000 | N.D. | 1.741 | 44 | 34770 |
| | 10000 | N.D. | | 71 | |
| | 2000 | 7 | | 77 | |
| | 400 | 9 | | 88 | |
| | 80 | 19 | | 99 | |
| | 16 | 44 | | 108 | |
| | 3.2 | 58 | | 108 | |
| | 0.64 | 60 | | 111 | |
| | 0.128 | 62 | | 113 | |
| | 0.026 | 64 | | 114 | |
| | 0.005 | 78 | | 105 | |
| | 0.001 | 96 | | 95 | |
| | 0.0002 | 97 | | N.D. | |
| | 0.00004 | 103 | | N.D. | |
| 1853612 | 50000 | N.D. | 1.746 | 63 | >50000 |
| | 10000 | N.D. | | 90 | |
| | 2000 | 10 | | 98 | |
| | 400 | 11 | | 105 | |
| | 80 | 21 | | 103 | |
| | 16 | 44 | | 112 | |
| | 3.2 | 59 | | 115 | |
| | 0.64 | 59 | | 105 | |
| | 0.128 | 60 | | 101 | |
| | 0.026 | 65 | | 103 | |
| | 0.005 | 75 | | 110 | |
| | 0.001 | 96 | | 90 | |
| | 0.0002 | 96 | | N.D. | |
| | 0.00004 | 104 | | N.D. | |
| 1859766 | 50000 | N.D. | 0.2677 | 66 | >50000 |
| | 10000 | N.D. | | 80 | |
| | 2000 | 5 | | 89 | |
| | 400 | 7 | | 93 | |
| | 80 | 15 | | 101 | |
| | 16 | 33 | | 100 | |
| | 3.2 | 45 | | 101 | |
| | 0.64 | 46 | | 97 | |
| | 0.128 | 50 | | 106 | |
| | 0.026 | 53 | | 105 | |
| | 0.005 | 64 | | 107 | |
| | 0.001 | 89 | | 93 | |
| | 0.0002 | 94 | | N.D. | |
| | 0.00004 | 106 | | N.D. | |
| 1853614 | 50000 | N.D. | 0.6451 | 56 | >50000 |
| | 10000 | N.D. | | 69 | |
| | 2000 | 6 | | 81 | |
| | 400 | 8 | | 85 | |
| | 80 | 16 | | 88 | |
| | 16 | 35 | | 93 | |
| | 3.2 | 52 | | 98 | |
| | 0.64 | 49 | | 90 | |
| | 0.128 | 54 | | 93 | |
| | 0.026 | 57 | | 102 | |
| | 0.005 | 72 | | 98 | |
| | 0.001 | 113 | | 102 | |
| | 0.0002 | 87 | | N.D. | |
| | 0.00004 | 101 | | N.D. | |

### Example 7: Effects of Oligomeric Duplexes Targeted to a Mouse TTR mRNA; 1-Week

Wild type C57BL/6 mice (Jackson Laboratory) were treated with oligomeric duplexes described in the above examples and evaluated for their effects on mouse TTR mRNA.

Groups of 4 male C57BL/6 mice each received a single subcutaneous injection of oligomeric duplex compound at various doses as indicated in the tables below. One group of 4 male C57BL/6 mice were injected with PBS and served as a negative control group.

7 days post treatment, mice were sacrificed and RNA was extracted from liver tissue for quantitative real-time RTPCR analysis of RNA expression of TTR using mouse primer probe set mTTR_1 (forward sequence CGTACTGGAAGACACTTGGCATT, designated herein as SEQ ID NO: 13; reverse sequence GAGTCGTTGGCTGTGAAAACC, designated herein as SEQ ID NO: 14; probe sequence CCCGTTCCATGAATTCGCGGATG, designated herein as SEQ ID NO: 15). TTR RNA levels were normalized to total RNA content, as measured by RIBOGREEN®. Results are presented as percent mouse TTR RNA relative to the amount of TTR RNA in PBS treated animals, (% control). The half maximal effective dose (ED₅₀) of each oligomeric duplex was calculated using GraphPad Prism 10 software (GraphPad Software, San Diego, CA).

TABLE 16
Reduction of mouse TTR RNA in wild type mice

| Compound | Dose | TTR RNA | ED50 |
|---|---|---|---|
| No. | (mg/kg) | (% control) | (mg/kg) |
| 1708616 | 0.1 | 47 | 0.09 |
| | 0.3 | 20 | |
| | 1 | 3 | |
| | 3 | 1 | |
| 1722757 | 0.1 | 56 | 0.13 |
| | 0.3 | 31 | |
| | 1 | 8 | |
| | 3 | 1 | |
| 1757524 | 0.1 | 48 | 0.10 |
| | 0.3 | 20 | |
| | 1 | 5 | |
| | 3 | 1 | |
| 1757527 | 0.1 | 54 | 0.12 |
| | 0.3 | 27 | |
| | 1 | 6 | |
| | 3 | 1 | |
| 1757530 | 0.1 | 63 | 0.16 |
| | 0.3 | 34 | |
| | 1 | 5 | |
| | 3 | 1 | |
| 1757533 | 0.1 | 70 | 0.16 |
| | 0.3 | 25 | |
| | 1 | 6 | |
| | 3 | 0.5 | |

### Example 8: Effects of Oligomeric Duplexes Targeted to a Mouse ACTN1 mRNA; 1-Week

Wild type C57BL/6 mice (Jackson Laboratory) were treated with oligomeric duplexes described in the above examples and evaluated for their effects on mouse ACTN1 mRNA.

Groups of 4 male C57BL/6 mice each received a single subcutaneous injection of oligomeric duplex compound at various doses as indicated in the tables below. One group of 4 male C57BL/6 mice were injected with PBS and served as a negative control group.

7 days post treatment, mice were sacrificed and RNA was extracted from liver tissue for quantitative real-time RTPCR analysis of RNA expression of ACTN1 using mouse primer probe set RTS3817 (forward sequence GATCCGGCCTGGGAGAAG, designated herein as SEQ ID NO: 18; reverse sequence TCTGTGTCCCCGCTTTGC, designated herein as SEQ ID NO: 19; probe sequence ACGTTCACAGCCTGGTGCAACTCCC, designated herein as SEQ ID NO: 20). ACTN1 RNA levels were normalized to total RNA content, as measured by RIBOGREEN®. Results are presented as percent mouse ACTN1 RNA relative to the amount of ACTN1 RNA in PBS treated animals, (% control). The half maximal effective dose (ED₅₀) of each oligomeric duplex was calculated using GraphPad Prism 10 software (GraphPad Software, San Diego, CA).

TABLE 17
Reduction of mouse ACTN1 RNA in wild type mice

| | | ACTN1 | |
|---|---|---|---|
| Compound | Dose | RNA | ED50 |
| No. | (mg/kg) | (% control) | (mg/kg) |
| 1744547 | 0.1 | 98 | 12.9 |
| | 0.3 | 74 | |
| | 1 | 56 | |
| | 3 | 46 | |
| | 10 | 52 | |
| | 30 | 49 | |
| | 100 | 42 | |
| 1788303 | 0.1 | 71 | 3.8 |
| | 0.3 | 65 | |
| | 1 | 58 | |
| | 3 | 40 | |
| | 10 | 40 | |
| | 30 | 43 | |
| | 100 | 41 | |
| 1788306 | 0.1 | 70 | 1.6 |
| | 0.3 | 64 | |
| | 1 | 43 | |
| | 3 | 38 | |
| | 10 | 46 | |
| | 30 | 34 | |
| | 100 | 32 | |

What is claimed:

1. An RNAi agent comprising an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, wherein at least one nucleoside of the seed region of the antisense RNAi oligonucleotide is a one stereo non-standard nucleoside having Formula X: 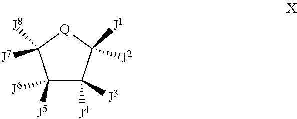
wherein one of J¹ and J² is H and the other is a heterocyclic base moiety Bx;
one of J³ and J⁴ is H and the other is a 2′-substituent selected from H, OH, OMe, F, and OCH₂CH₂OCH₃;
one of J⁵ and J⁶ is H and the other is —O-5′ linkage:
one of J⁷ and J⁸ is H and the other is —C(R¹)₂O-3′ linkage;
wherein each R¹ is independently H or C₁₋₆ alkyl;
provided that when J² and J⁸ are both H, then one of J³ and J⁵ is not H:
and provided that when Q is O and J² is H, then J⁸ is not H;
Q is O, S, or NR²;
wherein R² is H, C₁₋₆ alkyl, acetyl, or methanesulfonyl; and
Bx is a heterocyclic base moiety.

2. An RNAi agent for use in a method of comprising administering to a subject in need thereof a composition comprising the RNAi agent and a pharmaceutically acceptable carrier or diluent, optionally in a therapeutically effective amount thereof, wherein the RNAi agent comprises an antisense RNAi oligomeric compound comprising an antisense RNAi oligonucleotide consisting of 20-25 linked nucleosides and a sense RNAi oligomeric compound comprising a sense RNAi oligonucleotide consisting of 15-23 linked nucleosides, wherein at least one nucleoside of the seed region of the antisense RNAi oligonucleotide is a stereo non-standard nucleoside having Formula X: 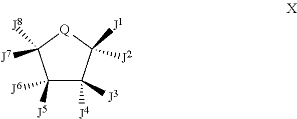
wherein one of J¹ and J² is H and the other is a heterocyclic base moiety Bx;
one of J³ and J⁴ is H and the other is a 2′-substituent selected from H, OH, OMe, F, and OCH₂CH₂OCH₃;
one of J⁵ and J⁶ is H and the other is —O-5′ linkage to the remainder of the oligonucleotide;
one of J⁷ and J⁸ is H and the other is —C(R¹)₂O-3′ linkage to the remainder of the oligonucleotide;
wherein each R¹ is independently H or C₁₋₆ alkyl;
provided that when Q is O and J² and J⁸ are both H, then one of J³ and J⁵ is not H;
Q is O, S, or NR²; and
wherein R² is H, C₁₋₆ alkyl, or acetyl.

3. The RNAi agent of claim 2, wherein the subject is a human.

4. The RNAi agent of claim 1 or 2, wherein the nucleoside having Formula X is one of nucleoside positions 3-8 of the antisense RNAi oligonucleotide, counting from the 5′-terminus thereof.

5. The RNAi agent of any of the preceding claims, wherein Q is O.

6. The RNAi agent of any of the preceding claims, wherein each R¹ is H.

7. The RNAi agent of any of the preceding claims, wherein each R² is H.

8. The RNAi agent of any of the preceding claims, wherein J² and J⁷ are each H.

9. The RNAi agent of any of the preceding claims, wherein J⁶ is H.

10. The RNAi agent of any of the preceding claims, wherein Bx is selected from uracil, thymine, cytosine, 5-methyl cytosine, adenine, guanine, and hypoxanthine.

11. The RNAi agent of any of the preceding claims, wherein J³ and J⁴ are each H.

12. The RNAi agent of any of claims 1 to 9, wherein one of J³ and J⁴ is H and the other is selected from OH, OMe, F, and OCH₂CH₂OCH₃.

13. The RNAi agent of any of claims 1 to 9, wherein one of J³ and J⁴ is H and the other is selected from OMe and F.

14. The RNAi agent of any of claims 1 to 9, wherein one of J³ and J⁴ is H and the other is F.

15. The RNAi agent of any of claims 1 to 9, wherein one of J³ and J⁴ is H and the other is OMe.

16. The RNAi agent of any of claims 1 to 9, wherein one of J³ and J⁴ is H and the other is OH.

17. The RNAi agent of any of the preceding claims, wherein the stereo-non-standard nucleoside has a structure selected from Formula I, Formula II, Formula III, Formula IV, Formula V, Formula VI, and Formula VII: 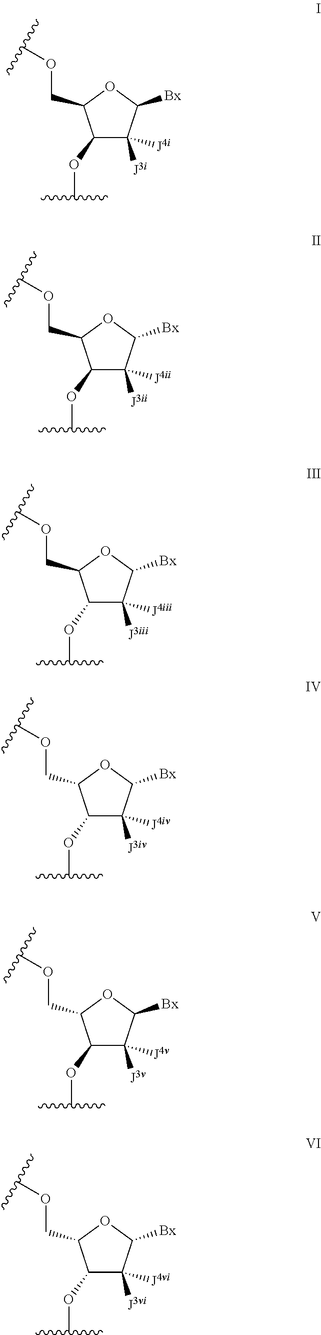 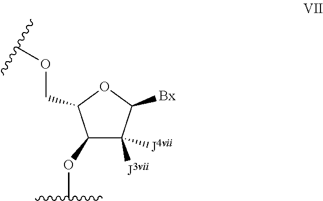
wherein J³ⁱ,³ⁱⁱ, J³ⁱⁱⁱ, J^{3iv}, J^{3v}, J^{3vi}, and J^{3vii} are as defined for J³ in Formula X; and J⁴ⁱ, J⁴ⁱⁱ, J⁴ⁱⁱⁱ, J^{4iv}, J^{4v}, J^{4vi}, and J^{4vii} are as defined for J⁴ in Formula X; and Bx is a heterocyclic base moiety.

18. The RNAi agent of any of the preceding claims, wherein the stereo-non-standard nucleoside has a structure of Formula I.

19. The RNAi agent of claim 17 or 18, wherein J³ⁱ and J⁴ⁱ are each H.

20. The RNAi agent of claim 17 or 18, wherein J³ⁱ is H and J⁴ⁱ is F.

21. The RNAi agent of claim 17 or 18, wherein J³ⁱ is H and J⁴ⁱ is OMe.

22. The RNAi agent of any of the preceding claims, wherein the stereo-non-standard nucleoside has a structure of Formula VII.

23. The RNAi agent of claim 17 or 22, wherein J^{3vii} and J^{4vii} are each H.

24. The RNAi agent of any of the preceding claims, wherein exactly one nucleoside of the antisense RNAi oligonucleotide is a stereo-non-standard nucleoside.

25. The RNAi agent of any of the preceding claims, wherein 2, 3, 4, 5, or 6 nucleosides of the antisense RNAi oligonucleotide are stereo-non-standard nucleosides.

26. The RNAi agent of any of the preceding claims, wherein nucleoside 6 from the 5′-terminus of the antisense RNAi oligonucleotide is a stereo-non-standard nucleoside, optionally wherein the remaining nucleosides in the antisense RNAi oligonucleotide are stereo-standard nucleosides.

27. The RNAi agent of any of the preceding claims, wherein nucleoside 3 from the 5′-terminus of the antisense RNAi oligonucleotide is a stereo-non-standard nucleoside, optionally wherein the remaining nucleosides in the antisense RNAi oligonucleotide are stereo-standard nucleosides.

28. The RNAi agent of any of the preceding claims, wherein nucleoside 4 from the 5′-terminus of the antisense RNAi oligonucleotide is a stereo-non-standard nucleoside, optionally wherein the remaining nucleosides in the antisense RNAi oligonucleotide are stereo-standard nucleosides.

29. The RNAi agent of any of the preceding claims, wherein nucleoside 5 from the 5′-terminus of the antisense RNAi oligonucleotide is a stereo-non-standard nucleoside, optionally wherein the remaining nucleosides in the antisense RNAi oligonucleotide are stereo-standard nucleosides.

30. The RNAi agent of any of the preceding claims, wherein nucleoside 7 from the 5′-terminus of the antisense RNAi oligonucleotide is a stereo-non-standard nucleoside, optionally wherein the remaining nucleosides in the antisense RNAi oligonucleotide are stereo-standard nucleosides.

31. The RNAi agent of any of the preceding claims, wherein nucleoside 8 from the 5′-terminus of the antisense RNAi oligonucleotide is a stereo-non-standard nucleoside, optionally wherein the remaining nucleosides in the antisense RNAi oligonucleotide are stereo-standard nucleosides.

32. The RNAi agent of any of the preceding claims, wherein each nucleoside in the sense RNAi oligonucleotide is a stereo-standard nucleoside.

33. The RNAi agent of any of the preceding claims, wherein the stereo-non-standard nucleoside is linked on at least one of the 3′- or 5′-position with a phosphodiester, phosphorothioate, or mesyl phosphoramidate internucleoside linkage.

34. The RNAi agent of any of the preceding claims, wherein the stereo-non-standard nucleoside is linked on at least one of the 3′- or 5′-position with a phosphodiester internucleoside linkage.

35. The RNAi agent of any of the preceding claims, wherein the stereo-non-standard nucleoside is linked at both the 3′- or 5′-positions with a phosphodiester internucleoside linkage.

36. The RNAi agent of any one of claims 1-34, wherein the stereo-non-standard nucleoside is linked on at least one of the 3′- or 5′-position with a phosphorothioate internucleoside linkage.

37. The RNAi agent of any of the preceding claims, wherein each remaining nucleoside in the antisense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside.

38. The RNAi agent of claim 32, wherein each bicyclic nucleoside is independently selected from an LNA nucleoside, a cEt nucleoside, and an ENA nucleoside.

39. The RNAi agent of any of the preceding claims, wherein each remaining nucleoside in the antisense RNAi oligonucleotide is a stereo-standard nucleoside independently selected from: a 2′-OMe nucleoside, a 2′-F nucleoside, a 2′-MOE nucleoside, and a 2′-deoxynucleoside.

40. The RNAi agent of any of the preceding claims, wherein at least one nucleoside in the antisense RNAi oligonucleotide is a 2′-OMe nucleoside.

41. The RNAi agent of claim 40, wherein 8-21 nucleosides in the antisense RNAi oligonucleotide are 2′-OMe nucleosides, optionally wherein 15-21 nucleosides in the antisense RNAi oligonucleotide are 2′-OMe nucleosides.

42. The RNAi agent of any of the preceding claims, wherein one, two, or three of the nucleosides at positions 3, 4, and 5 from the 5′-terminus of the antisense RNAi oligonucleotide are 2′-OMe nucleosides.

43. The RNAi agent of any of the preceding claims, wherein each of the nucleosides at positions 3, 4, 5 from the 5′-terminus of the antisense RNAi oligonucleotide are 2′-OMe nucleosides.

44. The RNAi agent of any of the preceding claims, wherein one, two, three, four, or five of the nucleosides at positions 7, 8, 9, 10, and 11 from the 5′-terminus of the antisense RNAi oligonucleotide are 2′-OMe nucleosides.

45. The RNAi agent of any of the preceding claims, wherein each of the nucleosides at positions 7, 8, 9, 10, and 11 from the 5′-terminus of the antisense RNAi oligonucleotide are 2′-OMe nucleosides.

46. The RNAi agent of any of the preceding claims, wherein one, two, three, four, or five of the nucleosides at positions 17, 18, 19, 20, and 21 from the 5′-terminus of the antisense RNAi oligonucleotide are 2′-OMe nucleosides.

47. The RNAi agent of any of the preceding claims, wherein each of the nucleosides at positions 17, 18, 19, 20, and 21 from the 5′-terminus of the antisense RNAi oligonucleotide are 2′-OMe nucleosides.

48. The RNAi agent of any of the preceding claims, wherein at least one nucleoside in the antisense RNAi oligonucleotide is a 2′-F nucleoside.

49. The RNAi agent of claim 48, wherein 1-10 nucleosides in the antisense RNAi oligonucleotide are 2′-F nucleosides, optionally wherein 1-6 nucleosides in the antisense RNAi oligonucleotide are 2′-F nucleosides, optionally wherein exactly 1, 2, 3, 4, 5, or 6 nucleosides in the antisense RNAi oligonucleotide are 2′-F nucleosides.

50. The RNAi agent of any of the preceding claims, wherein one, two, or three of the nucleosides at positions 2, 14, and 16 from the 5′-terminus of the antisense RNAi oligonucleotide are 2′-F nucleosides.

51. The RNAi agent of any of the preceding claims, wherein each of the nucleosides at positions 2, 14, and 16 from the 5′-terminus of the antisense RNAi oligonucleotide are 2′-F nucleosides.

52. The RNAi agent of any of the preceding claims, wherein the nucleoside at position 6 from the 5′-terminus of the antisense RNAi oligonucleotide is a 2′-F nucleoside.

53. The RNAi agent of any of the preceding claims, wherein the nucleoside at position 6 from the 5′-terminus of the antisense RNAi oligonucleotide is a 2′-OMe nucleoside.

54. The RNAi agent of any of the preceding claims, wherein at least one nucleoside in the antisense RNAi oligonucleotide is a 2′-MOE nucleoside.

55. The RNAi agent of claim 54, wherein 1-6 nucleosides in the antisense RNAi oligonucleotide are 2′-MOE nucleosides, optionally wherein 1-3 nucleosides in the antisense RNAi oligonucleotide are 2′-MOE nucleosides.

56. The RNAi agent of any of the preceding claims, wherein one, two, three, four, or five of the nucleosides at positions 1 from the 5′-terminus of the antisense RNAi oligonucleotide and positions 1, 2, 3, 4, or 5 from the 3′-terminus of the antisense RNAi oligonucleotide are 2′-MOE nucleosides: optionally wherein the antisense RNAi oligonucleotide consists of 23 linked nucleosides.

57. The RNAi agent of any of the preceding claims, wherein the nucleoside at position 1 from the 5′-terminus of the antisense RNAi oligonucleotide is a 2′-MOE nucleoside.

58. The RNAi agent of any of the preceding claims, wherein each of the nucleosides at positions 1 from the 5′-terminus of the antisense RNAi oligonucleotide and positions 1 and 2 from the 3′-end of the antisense RNAi oligonucleotide are 2′-MOE nucleosides.

59. The RNAi agent of any of the preceding claims, wherein one or two of the nucleosides at positions 9 and 10 from the 5′-terminus of the antisense RNAi oligonucleotide are 2′-MOE nucleosides.

60. The RNAi agent of any of the preceding claims, wherein the 5′-terminal nucleoside of the antisense RNAi oligonucleotide comprises a stabilized phosphate group.

61. The RNAi agent of claim 51, wherein the stabilized phosphate group is 5′-vinyl phosphonate, optionally E-5′-vinyl phosphonate.

62. The RNAi agent of claim 51, wherein the stabilized phosphate group is 5′-cyclopropyl phosphonate.

63. The RNAi agent of any of the preceding claims, wherein the antisense RNAi oligonucleotide consists of 21-23, optionally 23, linked nucleosides.

64. The RNAi agent of any of the preceding claims, wherein each nucleoside in the sense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside.

65. The RNAi agent of claim 55, wherein each bicyclic nucleoside is independently selected from an LNA nucleoside, a cEt nucleoside, and an ENA nucleoside.

66. The RNAi agent of any of the preceding claims, wherein each nucleoside in the sense RNAi oligonucleotide is a stereo-standard nucleoside independently selected from: a 2′-OMc nucleoside, a 2′-F nucleoside, a 2′-MOE nucleoside, and a 2′-deoxynucleoside.

67. The RNAi agent of any of the preceding claims, wherein at least one nucleoside in the sense RNAi oligonucleotide is a 2′-OMe nucleoside.

68. The RNAi agent of claim 67, wherein 8-21 nucleosides in the sense RNAi oligonucleotide are 2′-OMe nucleosides, optionally wherein 13-21 nucleosides in the sense RNAi oligonucleotide are 2′-OMe nucleosides.

69. The RNAi agent of any of the preceding claims, wherein at least one of the nucleosides at positions 3-6 from the 5′-terminus of the sense RNAi oligonucleotide are 2′-OMe nucleosides, optionally wherein each of the nucleosides at positions 3-8 is a 2′-OMe nucleoside.

70. The RNAi agent of any of the preceding claims, wherein at least one of the nucleosides at positions 12-19 from the 5′-terminus of the sense RNAi oligonucleotide are 2′-OMe nucleosides, optionally wherein each of the nucleosides at positions 12-19 is a 2′-OMe nucleoside.

71. The RNAi agent of any of the preceding claims, wherein at least one nucleoside in the sense RNAi oligonucleotide is a 2′-F nucleoside.

72. The RNAi agent of claim 71, wherein 1-11 nucleosides in the sense RNAi oligonucleotide are 2′-F nucleosides, optionally wherein 2-4 nucleosides in the sense RNAi oligonucleotide are 2′-F nucleosides, optionally wherein exactly 2, 3, or 4 nucleosides in the sense RNAi oligonucleotide are 2′-F nucleosides.

73. The RNAi agent of any of the preceding claims, wherein one, two, three, or four of the nucleosides at positions 7 and 9-11 from the 5′-terminus of the sense RNAi oligonucleotide are 2′-F nucleosides, optionally wherein each of the nucleosides at positions 7 and 9-11 is a 2′-F nucleoside.

74. The RNAi agent of any of the preceding claims, wherein at least one nucleoside in the sense RNAi oligonucleotide is a 2′-deoxynucleoside.

75. The RNAi agent of any of the preceding claims, wherein one, two, three, four, or five of the nucleosides at positions 7-11 from the 5′-terminus of the sense RNAi oligonucleotide are 2′-deoxynucleosides, optionally wherein one of the nucleosides at positions 7-11 is a 2′-deoxynucleoside.

76. The RNAi agent of any of the preceding claims, wherein at least one nucleoside in the sense RNAi oligonucleotide is a 2′-MOE nucleoside.

77. The RNAi agent of claim 71, wherein 1-6 nucleosides in the sense RNAi oligonucleotide are 2′-MOE nucleosides, optionally wherein 2-4 nucleosides in the sense RNAi oligonucleotide are 2′-MOE nucleosides.

78. The RNAi agent of any of the preceding claims, wherein one, two, three, or four of the nucleosides at positions 1, 2, 20, and 21 from the 5′-terminus of the sense RNAi oligonucleotide are 2′-MOE nucleosides, optionally wherein each of positions 1, 2, 20, and 21 is a 2′-MOE nucleoside; optionally wherein the sense RNAi oligonucleotide consists of 21 linked nucleosides.

79. The RNAi agent of any of the preceding claims, wherein one, two, three, or four of the nucleosides at positions 1, 2, 18, 19 from the 5′-terminus of the sense RNAi oligonucleotide are 2′-MOE nucleosides, optionally wherein each of positions 1, 2, 18, and 19 is a 2′-MOE nucleoside: optionally wherein the sense RNAi oligonucleotide consists of 19 linked nucleosides.

80. The RNAi agent of any of the preceding claims, wherein at least one nucleoside in the sense RNAi oligonucleotide is substituted at its 2′-position with a C₁₆-C₂₂ alkyl group, optionally wherein the C₁₆-C₂₂ alkyl group is unsubstituted, optionally wherein the C₁₆-C₂₂ alkyl group is saturated.

81. The RNAi agent of any of the preceding claims, wherein the sense RNAi oligonucleotide consists of 14-21 linked nucleosides.

82. The RNAi agent of any of the preceding claims, wherein the sense RNAi oligonucleotide consists of 19-21, optionally 21, linked nucleosides.

83. The RNAi agent of any of the preceding claims, wherein the antisense RNAi oligonucleotide comprises one, two, three, or four phosphorothioate internucleoside linkages joining nucleosides at positions selected from 1 and 2, 2 and 3 from the 5′-terminus thereof, and positions 1 and 2, 2 and 3 from the 3′-terminus thereof.

84. The RNAi agent of claim 83, wherein the antisense RNAi oligonucleotide comprises four phosphorothioate internucleoside linkages.

85. The RNAi agent of any of the preceding claims, wherein the sense RNAi oligonucleotide comprises one, two, three, or four phosphorothioate internucleoside linkages joining nucleosides at positions selected from 1 and 2, 2 and 3 from the 5′-terminus thereof, and positions 1 and 2, 2 and 3 from the 3′-terminus thereof.

86. The RNAi agent of claim 85, wherein the sense RNAi oligonucleotide comprises exactly two phosphorothioate internucleoside linkages.

87. The RNAi agent of any of the preceding claims, wherein the antisense RNAi oligonucleotide comprises an overhang of two nucleosides at the 3′-terminus thereof.

88. The RNAi agent of any of the preceding claims, wherein each overhanging nucleoside comprises a 2′-MOE sugar moiety or a 2′-deoxy sugar moiety.

89. The RNAi agent of any of the preceding claims, wherein the antisense siRNA oligonucleotide has a nucleobase sequence comprising a targeting region comprising at least 15, optionally 15-21, contiguous nucleobases, wherein the nucleobase sequence of targeting region is at least 85% complementary to an equal length portion of the nucleobase sequence of a target RNA.

90. The RNAi agent of claim 89, wherein the nucleobase sequence of the targeting region is at least 90% complementary to the target RNA.

91. The RNAi agent of claim 89, wherein the nucleobase sequence of the targeting region is at least 95% complementary to the target RNA.

92. The RNAi agent of claim 89, wherein the nucleobase sequence of the targeting region is 100% complementary to the target RNA.

93. The RNAi agent of any of claims 89-92, wherein the target RNA encodes a protein.

94. The RNAi agent of any of the preceding claims, wherein the sense siRNA oligonucleotide is sufficiently complementary to the antisense siRNA oligonucleotide to fonn a stable duplex at room temperature in water.

95. The RNAi agent of any of the preceding claims, wherein the antisense RNAi oligonucleotide consists of 21 linked nucleosides.

96. The RNAi agent of any of the preceding claims, wherein the antisense RNAi oligonucleotide consists of 23 linked nucleosides.

97. The RNAi agent of any of the preceding claims, wherein the sense RNAi oligonucleotide consists of 19 linked nucleosides.

98. The RNAi agent of any of the preceding claims, wherein the sense RNAi oligonucleotide consists of 21 linked nucleosides.

99. The RNAi agent of any of the preceding claims, wherein the RNAi agent comprises a conjugate group.

100. The RNAi agent of claim 99, wherein the conjugate group comprises a cell-targeting moiety.

101. The RNAi agent of claim 100, wherein the cell-targeting moiety has affinity for an ASGPR receptor or a Tfr1 receptor.

102. An RNAi agent comprising an antisense RNAi oligomeric compound comprising an antisense RNAi oligonucleotide consisting of 20-50 linked nucleosides and optionally a sense RNAi oligomeric compound comprising a sense RNAi oligonucleotide consisting of 15-23 linked nucleosides, wherein the antisense RNAi oligonucleotide comprises at one or more of nucleosides 3-8 from the 5′-terminus thereof a nucleoside having Formula I: 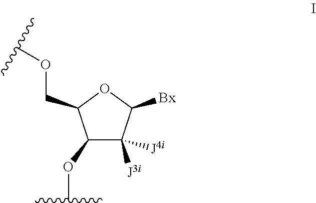
wherein J³ⁱ is H and J⁴ⁱ is selected from H, OH, OMe, F, and OCH₂CH₂OCH₃;
and Bx is a heterocyclic base moiety;
provided that the RNAi agent does not comprise any of the following: 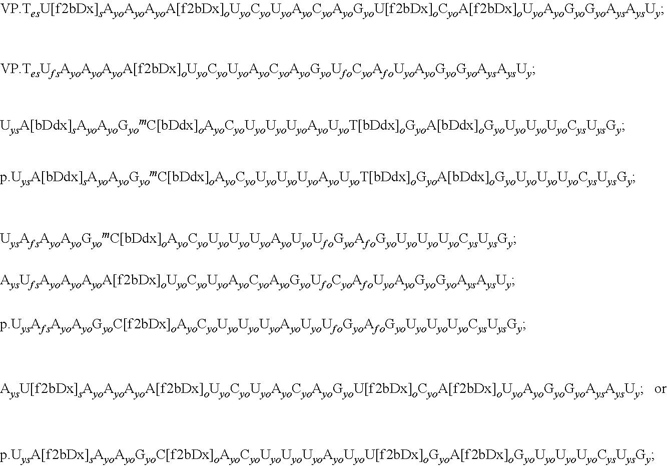
wherein “VP” represents a 5′-vinyl phosphonate moiety, “p” represents a 5′-phosphate moiety, a subscript “[f2bDx]” represents a 2′-fluoro-β-D-xylosyl sugar moiety, a subscript “f” represents a 2′-fluoro-β-D-ribosyl sugar moiety, a subscript “y” represents a 2′-O-methyl-β-D-ribosyl sugar moiety, a subscript “e” represents a 2′-MOE-β-D-ribosyl sugar moiety, a superscript “m” represents a 5-methyl group on cytosine, a subscript “s” represents a phosphorothioate internucleoside linkage, and a subscript “o” represents a phosphodiester internucleoside linkage.

103. The RNAi agent of claim 102, wherein J⁴ⁱ is H.

104. The RNAi agent of claim 102, wherein J⁴ⁱ is F.

105. The RNAi agent of claim 102, wherein J⁴ⁱ is OMe.

106. The RNAi agent of any claims 102-105, wherein exactly one nucleoside of the antisense RNAi oligonucleotide is of Formula T.

107. The RNAi agent of any of claims 102-105, wherein 2, 3, 4, 5, or 6 nucleosides of the antisense RNAi oligonucleotide are of Formula I.

108. The RNAi agent of any of claims 102-107, wherein nucleoside 6 from the 5′-terminus of the antisense RNAi oligonucleotide is a nucleoside of Formula I, optionally wherein the remaining nucleosides in the antisense RNAi oligonucleotide are stereo-standard nucleosides.

109. The RNAi agent of any of claims 102-108, wherein nucleoside 3 from the 5′-terminus of the antisense RNAi oligonucleotide is a nucleoside of Formula I, optionally wherein the remaining nucleosides in the antisense RNAi oligonucleotide are stereo-standard nucleosides.

110. The RNAi agent of any of claims 102-109, wherein nucleoside 4 from the 5′-terminus of the antisense RNAi oligonucleotide is a nucleoside of Formula I, optionally wherein the remaining nucleosides in the antisense RNAi oligonucleotide are stereo-standard nucleosides.

111. The RNAi agent of any of claims 102-110, wherein nucleoside 5 from the 5′-terminus of the antisense RNAi oligonucleotide is a nucleoside of Formula I, optionally wherein the remaining nucleosides in the antisense RNAi oligonucleotide are stereo-standard nucleosides.

112. The RNAi agent of any of claims 102-111, wherein nucleoside 7 from the 5′-terminus of the antisense RNAi oligonucleotide is a nucleoside of Formula I, optionally wherein the remaining nucleosides in the antisense RNAi oligonucleotide are stereo-standard nucleosides.

113. The RNAi agent of any of claims 102-112, wherein nucleoside 8 from the 5′-terminus of the antisense RNAi oligonucleotide is a nucleoside of Formula I, optionally wherein the remaining nucleosides in the antisense RNAi oligonucleotide are stereo-standard nucleosides.

114. The RNAi agent of any of claims 102-113, wherein each nucleoside in the sense RNAi oligonucleotide is a stereo-standard nucleoside.

115. The RNAi agent of any of claims 102-114, wherein the nucleoside of Formula I is linked on at least one of the 3′- or 5′-position with a phosphodiester, phosphorothioate, or mesyl phosphoramidate internucleoside linkage.

116. The RNAi agent of any of claims 102-115, wherein the nucleoside of Formula I is linked on at least one of the 3′- or 5′-position with a phosphodiester internucleoside linkage.

117. The RNAi agent of any of claims 102-116, wherein the nucleoside of Formula I is linked at both the 3′- and 5′-positions with a phosphodiester internucleoside linkage.

118. The RNAi agent of any of claims 102-116, wherein the nucleoside of Formula I is linked on at least one of the 3′- or 5′-position with a phosphorothioate internucleoside linkage.

119. The RNAi agent of any of claim 102-118, wherein each remaining nucleoside in the antisense RNAi oligonucleotide is independently selected from: a bicyclic nucleoside, a 2′-OMc nucleoside, a 2′-F nucleoside, a 2-MOE nucleoside, a 2′-NMA nucleoside, a 2′-deoxynucleoside, and a ribosyl nucleoside.

120. The RNAi agent of any of claims 102-119, wherein each remaining nucleoside in the antisense RNAi oligonucleotide is a stereo-standard nucleoside independently selected from: a 2′-OMe nucleoside, a 2′-F nucleoside, a 2′-MOE nucleoside, and a 2′-deoxynucleoside.

121. The RNAi agent of any of claims 102-120, wherein the RNAi agent does not comprise a stereo-non-standard nucleoside outside of positions 3-8 from the 5′-terminus of the antisense RNAi oligonucleotide.

122. The RNAi agent of any of claims 102-121, wherein J⁴ⁱ is F and the nucleoside of Formula I is at position 5 or 7 from the 5′-terminus of the antisense RNAi oligonucleotide.

123. The RNAi agent of any of claims 102-121 for use in treatment of a disease or condition, wherein J⁴ⁱ is F and the nucleoside of Formula I is at position 6 from the 5′-terminus of the antisense RNAi oligonucleotide.

124. The RNAi agent of any of claim 123, wherein the treatment comprises administering to a subject in need thereof a composition comprising the RNAi agent and a pharmaceutically acceptable carrier or diluent, optionally in a therapeutically effective amount thereof, optionally wherein the subject is a human.

125. The RNAi agent of any of claims 102-121, wherein J⁴ⁱ is H and the nucleoside of Formula I is at position 5 or 7 from the 5′-terminus of the antisense RNAi oligonucleotide.

126. The RNAi agent of any of claims 102-121 for use in treatment of a disease or condition, wherein J⁴ⁱ is H and the nucleoside of Formula I is at position 6 from the 5′-terminus of the antisense RNAi oligonucleotide.

127. The RNAi agent of any of claim 126, wherein the treatment comprises administering to a subject in need thereof a composition comprising the RNAi agent and a pharmaceutically acceptable carrier or diluent, optionally in a therapeutically effective amount thereof, optionally wherein the subject is a human.

128. The RNAi agent of any of claims 102-121, wherein J⁴ⁱ is OMe and the nucleoside of Formula I is at position 5, 6, or 7 from the 5′-terminus of the antisense RNAi oligonucleotide.

129. The RNAi agent of any of claim 128, wherein the compound is for use in treatment of a disease or condition, optionally wherein the treatment comprises administering to a subject in need thereof a composition comprising the RNAi agent and a pharmaceutically acceptable carrier or diluent, optionally in a therapeutically effective amount thereof, optionally wherein the subject is a human.

130. The RNAi agent of any of claims 102-129, wherein the 5′-terminal nucleoside of the antisense RNAi oligonucleotide comprises a stabilized phosphate group.

131. The RNAi agent of claim 130, wherein the stabilized phosphate group is 5′-vinyl phosphonate, optionally E-5′-vinyl phosphonate.

132. The RNAi agent of claim 130, wherein the stabilized phosphate group is 5′-cyclopropyl phosphonate.

133. The RNAi agent of any of claims 102-132, wherein the antisense RNAi oligonucleotide consists of 21-23, optionally 23, linked nucleosides.

134. The RNAi agent of any of claims 102-133, wherein the sense RNAi oligonucleotide consists of 14-21 linked nucleosides.

135. The RNAi agent of any of claims 102-134, wherein the sense RNAi oligonucleotide consists of 19-21, optionally 21, linked nucleosides.

136. The RNAi agent of any of claims 102-135, wherein Bx is selected from uracil, thymine, cytosine, 5-methyl cytosine, adenine, guanine, and hypoxanthine.

137. The RNAi agent of any of claims 102-135, wherein Bx is selected from uracil, thymine, cytosine, adenine, guanine, and hypoxanthine.

138. The RNAi agent of any of claims 102-135, wherein Bx is selected from uracil, thymine, cytosine, adenine, and guanine.

139. The RNAi agent of any of claims 102-138, wherein each stereo-standard nucleoside in the antisense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside.

140. The RNAi agent of claim 139, wherein each bicyclic nucleoside is independently selected from an LNA nucleoside, a cEt nucleoside, and an ENA nucleoside.

141. The RNAi agent of any of claims 102-140, wherein each remaining nucleoside in the antisense RNAi oligonucleotide is a stereo-standard nucleoside independently selected from: a 2′-OMe nucleoside, a 2′-F nucleoside, a 2′-MOE nucleoside, and a 2′-deoxynucleoside.

142. The RNAi agent of any of claims 102-141, wherein at least one nucleoside in the antisense RNAi oligonucleotide is a 2′-OMe nucleoside.

143. The RNAi agent of any of claims 102-142, wherein 8-21 nucleosides in the antisense RNAi oligonucleotide are 2′-OMe nucleosides, optionally wherein 15-21 nucleosides in the antisense RNAi oligonucleotide are 2′-OMe nucleosides.

144. The RNAi agent of any of claims 102-143, wherein the nucleoside at position 1 from the 5′-terminus of the antisense RNAi oligonucleotide is a 2′-OMe nucleoside.

145. The RNAi agent of any of claims 102-144, wherein one, two, or three of the nucleosides at positions 3, 4, and 5 from the 5′-terminus of the antisense RNAi oligonucleotide are 2′-OMe nucleosides.

146. The RNAi agent of any of claims 102-145, wherein each of the nucleosides at positions 3, 4, 5 from the 5′-terminus of the antisense RNAi oligonucleotide are 2′-OMe nucleosides.

147. The RNAi agent of any of claims 102-146, wherein one, two, three, four, or five of the nucleosides at positions 7, 8, 9, 10, and 11 from the 5′-terminus of the antisense RNAi oligonucleotide are 2′-OMe nucleosides.

148. The RNAi agent of any of claims 102-147, wherein each of the nucleosides at positions 7, 8, 9, 10, and 11 from the 5′-terminus of the antisense RNAi oligonucleotide are 2′-OMe nucleosides.

149. The RNAi agent of any of claims 102-148, wherein one, two, three, four, or five of the nucleosides at positions 17, 18, 19, 20, and 21 from the 5′-terminus of the antisense RNAi oligonucleotide are 2′-OMe nucleosides.

150. The RNAi agent of any of claims 102-149, wherein each of the nucleosides at positions 17, 18, 19, 20, and 21 from the 5′-terminus of the antisense RNAi oligonucleotide are 2′-OMe nucleosides.

151. The RNAi agent of any of claims 102-150, wherein at least one nucleoside in the antisense RNAi oligonucleotide is a 2′-F nucleoside.

152. The RNAi agent of claim 151, wherein 1-10 nucleosides in the antisense RNAi oligonucleotide are 2′-F nucleosides, optionally wherein 1-6 nucleosides in the antisense RNAi oligonucleotide are 2′-F nucleosides, optionally wherein exactly 1, 2, 3, 4, 5, or 6 nucleosides in the antisense RNAi oligonucleotide are 2′-F nucleosides.

153. The RNAi agent of any of claims 102-152, wherein one, two, or three of the nucleosides at positions 2, 14, and 16 from the 5′-terminus of the antisense RNAi oligonucleotide are 2′-F nucleosides.

154. The RNAi agent of any of claims 102-153, wherein each of the nucleosides at positions 2, 14, and 16 from the 5′-terminus of the antisense RNAi oligonucleotide are 2′-F nucleosides.

155. The RNAi agent of any of claims 102-154, wherein the nucleoside at position 6 from the 5′-terminus of the antisense RNAi oligonucleotide is a 2′-F nucleoside.

156. The RNAi agent of any of claims 102-154, wherein the nucleoside at position 6 from the 5′-terminus of the antisense RNAi oligonucleotide is a 2′-OMe nucleoside.

157. The RNAi agent of any of claims 102-156, wherein at least one nucleoside in the antisense RNAi oligonucleotide is a 2′-MOE nucleoside.

158. The RNAi agent of claim 157, wherein 1-6 nucleosides in the antisense RNAi oligonucleotide are 2′-MOE nucleosides, optionally wherein 1-3 nucleosides in the antisense RNAi oligonucleotide are 2′-MOE nucleosides.

159. The RNAi agent of any of claims 102-158, wherein one, two, three, four, or five of the nucleosides at positions 1 from the 5′-terminus of the antisense RNAi oligonucleotide and positions 1, 2, 3, 4, or 5 from the 3′-terminus of the antisense RNAi oligonucleotide are 2′-MOE nucleosides.

160. The RNAi agent of any of claims 102-159, wherein the nucleoside at position 1 from the 5′-terminus of the antisense RNAi oligonucleotide is a 2′-MOE nucleoside.

161. The RNAi agent of any of claims 102-160, wherein each of the nucleosides at positions 1 from the 5′-terminus of the antisense RNAi oligonucleotide and positions 1 and 2 from the 3′-end of the antisense RNAi oligonucleotide are 2′-MOE nucleosides.

162. The RNAi agent of any of claims 102-161, wherein one or two of the nucleosides at positions 9 and 10 from the 5′-terminus of the antisense RNAi oligonucleotide are 2′-MOE nucleosides.

163. The RNAi agent of any of claims 102-162, wherein the 5′-terminal nucleoside of the antisense RNAi oligonucleotide comprises a stabilized phosphate group.

164. The RNAi agent of claim 163, wherein the stabilized phosphate group is 5′-vinyl phosphonate, optionally E-5′-vinyl phosphonate.

165. The RNAi agent of claim 163, wherein the stabilized phosphate group is 5′-cyclopropyl phosphonate.

166. The RNAi agent of any of claims 102-165, wherein the antisense RNAi oligonucleotide consists of 21-23, optionally 23, linked nucleosides.

167. The RNAi agent of any of claims 102-166, wherein each nucleoside in the sense RNAi oligonucleotide is independently selected from: a bicyclic nucleoside, a 2′-OMe nucleoside, a 2′-F nucleoside, a 2′-MOE nucleoside, a 2′-NMA nucleoside, a 2′-deoxynucleoside, and a ribosyl nucleoside.

168. The RNAi agent of claim 167, wherein each bicyclic nucleoside is independently selected from an LNA nucleoside, a cEt nucleoside, and an ENA nucleoside.

169. The RNAi agent of any of claims 102-168, wherein each nucleoside in the sense RNAi oligonucleotide is a stereo-standard nucleoside independently selected from: a 2′-OMe nucleoside, a 2′-F nucleoside, a 2′-MOE nucleoside, and a 2′-deoxynucleoside.

170. The RNAi agent of any of claims 102-169, wherein at least one nucleoside in the sense RNAi oligonucleotide is a 2′-OMe nucleoside.

171. The RNAi agent of claim 170, wherein 8-21 nucleosides in the sense RNAi oligonucleotide are 2′-OMe nucleosides, optionally wherein 13-21 nucleosides in the sense RNAi oligonucleotide are 2′-OMe nucleosides.

172. The RNAi agent of any of claims 102-171, wherein at least one of the nucleosides at positions 3-6 from the 5′-terminus of the sense RNAi oligonucleotide are 2′-OMe nucleosides, optionally wherein each of the nucleosides at positions 3-8 is a 2′-OMe nucleoside.

173. The RNAi agent of any of claims 102-172, wherein at least one of the nucleosides at positions 12-19 from the 5′-terminus of the sense RNAi oligonucleotide are 2′-OMc nucleosides, optionally wherein each of the nucleosides at positions 12-19 is a 2′-OMe nucleoside.

174. The RNAi agent of any of claims 102-173, wherein at least one nucleoside in the sense RNAi oligonucleotide is a 2′-F nucleoside.

175. The RNAi agent of claim 174, wherein 1-11 nucleosides in the sense RNAi oligonucleotide are 2′-F nucleosides, optionally wherein 2-4 nucleosides in the sense RNAi oligonucleotide are 2′-F nucleosides, optionally wherein exactly 2, 3, or 4 nucleosides in the sense RNAi oligonucleotide are 2′-F nucleosides.

176. The RNAi agent of any of claims 102-175, wherein one, two, three, or four of the nucleosides at positions 7 and 9-11 from the 5′-terminus of the sense RNAi oligonucleotide are 2′-F nucleosides, optionally wherein each of the nucleosides at positions 7 and 9-11 is a 2′-F nucleoside.

177. The RNAi agent of any of claims 102-176, wherein at least one nucleoside in the sense RNAi oligonucleotide is a 2′-deoxynucleoside.

178. The RNAi agent of any of claims 102-177, wherein one, two, three, four, or five of the nucleosides at positions 7-11 from the 5′-terminus of the sense RNAi oligonucleotide are 2′-deoxynucleosides, optionally wherein one of the nucleosides at positions 7-11 is a 2′-deoxynucleoside.

179. The RNAi agent of any of claims 102-178, wherein at least one nucleoside in the sense RNAi oligonucleotide is a 2′-MOE nucleoside.

180. The RNAi agent of claim 179, wherein 1-6 nucleosides in the sense RNAi oligonucleotide are 2′-MOE nucleosides, optionally wherein 2-4 nucleosides in the sense RNAi oligonucleotide are 2′-MOE nucleosides.

181. The RNAi agent of any of claims 102-180, wherein one, two, three, or four of the nucleosides at positions 1, 2, 20, and 21 from the 5′-terminus of the antisense RNAi oligonucleotide are 2′-MOE nucleosides, optionally wherein each of positions 1, 2, 20, and 21 is a 2′-MOE nucleoside.

182. The RNAi agent of any of claims 102-181, wherein one, two, three, or four of the nucleosides at positions 1, 2, 18, 19 from the 5′-terminus of the antisense RNAi oligonucleotide are 2′-MOE nucleosides, optionally wherein each of positions 1, 2, 18, and 19 is a 2′-MOE nucleoside.

183. The RNAi agent of any of claims 102-182, wherein at least one nucleoside in the sense RNAi oligonucleotide is substituted at its 2′-position with a C₁₆-C₂₂ alkyl group, optionally wherein the C₁₆-C₂₂ alkyl group is unsubstituted, optionally wherein the C₁₆-C₂₂ alkyl group is saturated.

184. The RNAi agent of any of claims 102-183, wherein the RNAi agent comprises a hairpin.

185. The RNAi agent of any of claims 102-184, wherein the RNAi agent comprises a tetraloop hairpin.

186. A population of RNAi agents of any of the preceding claims, wherein the stereo-non-standard nucleoside is chirally enriched for internucleoside linkages having the Rp or Sp configuration.

187. A population of RNAi agents of any of claims 1-185, wherein the stereochemical configuration at each internucleoside linkage is stereorandom.

188. A pharmaceutical composition comprising the RNAi agent or population of any of claims 1-187 and a pharmaceutically acceptable carrier or diluent.

189. A method comprising contacting a cell with the RNAi agent or population of any of claims 1-187 or pharmaceutical composition of claim 188.

190. A method of modulating the amount or activity of a target nucleic acid in a cell, comprising contacting the cell with the RNAi agent or population of any of claims 1-187 or pharmaceutical composition of claim 188.

191. The method of claim 190, wherein the amount or activity of a target nucleic acid is reduced.

192. Use of the RNAi agent, population, or composition of any of claims 1-188 for treatment of a disease or condition.

193. Use of the RNAi agent, population, or composition of any of claims 1-188 in therapy.

194. The RNAi agent, population, or composition of any of claims 1-188 for use in preparation of a medicament for treatment of a disease or condition.

195. The method or the use of any of claims 2-194, wherein the RNAi agent has reduced off-target effect compared to an RNAi agent having a stereo-standard nucleoside of the same 2′-substituent at the position of each nucleoside of Formula X.

196. The method or use of claim 195, wherein reduced off-target effect is determined by IC50 for knockdown of one or more off-target genes.

197. The method or use of claim 195, wherein reduced off-target effect is determined by number of differentially expressed off-target genes.


SEQUENCE LISTING (21 sequences)

SEQ ID NO: 1 - RNA, 1237 nt, Mus musculus
  source 1..1237: mol_type = mRNA
ctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggatggcttcccttcgactcttcctcctttgcctcgctggactggtatttgtgtctgaagctggccccgcgggtgctggagaatccaaatgtcctctgatggtcaaagtcctggatgctgtccgaggcagccctgctgtagacgtggctgtaaaagtgttcaaaaagacctctgagggatcctgggagccctttgcctctgggaagaccgcggagtctggagagctgcacgggctcaccacagatgagaagtttgtagaaggagtgtacagagtagaactggacaccaaatcgtactggaagacacttggcatttccccgttccatgaattcgcggatgtggttttcacagccaacgactctggccatcgccactacaccatcgcagccctgctcagcccatactcctacagcaccacggctgtcgtcagcaacccccagaattgagagactcagcccaggaggaccaggatcttgccaaagcagtagcatcccatttgtaccaaaacagtgttcttgctctataaaccgtgttagcagctcaggaagatgccgtgaagcattcttattaaaccacctgctatttcattcaaactgtgtttcttttttatttcctcatttttctcccctgctcctaaaacccaaaatcttctaaagaattctagaaggtatgcgatcaaactttttaaagaaagaaaatactttttgactcatggtttaaaggcatcctttccatcttggggaggtcatgggtgctcctggcaacttgcttgaggaagataggtcagaaagcagagtggaccaaccgttcaatgttttacaagcaaaacatacactaagcatggtctgtagctattaaaagcacacaatctgaagggctgtagatgcacagtagtgttttcccagagcatgttcaaaagccctgggttcaatcacaatactgaaaagtaggccaaaaaacattctgaaaatgaaatatttgggtttttttttataacctttagtgactaaataaagacaaatctaagagactaaaaaaaaaaaaaaaaaa

SEQ ID NO: 2 - RNA, 23 nt, synthetic construct
  source 1..23: mol_type = other RNA
  modified_base 1: mod_base = OTHER; note = thymine
ttatagagcaagaacactgtttt

SEQ ID NO: 3 - RNA, 21 nt, synthetic construct
  source 1..21: mol_type = other RNA
aacagtgttcttgctctataa

SEQ ID NO: 4 - DNA, 23 bp, synthetic construct
  source 1..23: mol_type = other DNA
aaaacagtgttcttgctctataa

SEQ ID NO: 5 - DNA, 19 bp, synthetic construct
  source 1..19: mol_type = other DNA
taatattacataaataaaa

SEQ ID NO: 6 - RNA, 23 nt, synthetic construct
  source 1..23: mol_type = other RNA
  modified_base 1: mod_base = OTHER; note = thymine
taagtgagctagcaaacacacat

SEQ ID NO: 7 - RNA, 23 nt, synthetic construct
  source 1..23: mol_type = other RNA
caagtgagctagcaaacacacat

SEQ ID NO: 8 - RNA, 23 nt, synthetic construct
  source 1..23: mol_type = other RNA
  modified_base 1: mod_base = OTHER; note = thymine
taattttgtaaactgtcactttg

SEQ ID NO: 9 - RNA, 23 nt, synthetic construct
  source 1..23: mol_type = other RNA
  modified_base 1: mod_base = OTHER; note = thymine
  modified_base 6: mod_base = OTHER; note = thymine
taattttgtaaactgtcactttg

SEQ ID NO: 10 - RNA, 21 nt, synthetic construct
  source 1..21: mol_type = other RNA
gtgtgtttgctagctcactta

SEQ ID NO: 11 - RNA, 21 nt, synthetic construct
  source 1..21: mol_type = other RNA
gtgtgtttgctagctcacttg

SEQ ID NO: 12 - RNA, 21 nt, synthetic construct
  source 1..21: mol_type = other RNA
aagtgacagtttacaaaatta

SEQ ID NO: 13 - DNA, 23 bp, synthetic construct
  source 1..23: mol_type = other DNA
cgtactggaagacacttggcatt

SEQ ID NO: 14 - DNA, 21 bp, synthetic construct
  source 1..21: mol_type = other DNA
gagtcgttggctgtgaaaacc

SEQ ID NO: 15 - DNA, 23 bp, synthetic construct
  source 1..23: mol_type = other DNA
cccgttccatgaattcgcggatg

SEQ ID NO: 16 - DNA, 23 bp, synthetic construct
  source 1..23: mol_type = other DNA
atgtgtgtttgctagctcactta

SEQ ID NO: 17 - DNA, 19 bp, synthetic construct
  source 1..19: mol_type = other DNA
taatattacaaaaataaat

SEQ ID NO: 18 - DNA, 18 bp, synthetic construct
  source 1..18: mol_type = other DNA
gatccggcctgggagaag

SEQ ID NO: 19 - DNA, 18 bp, synthetic construct
  source 1..18: mol_type = other DNA
tctgtgtccccgctttgc

SEQ ID NO: 20 - DNA, 25 bp, synthetic construct
  source 1..25: mol_type = other DNA
acgttcacagcctggtgcaactccc

SEQ ID NO: 21 - RNA, 3739 nt, Mus musculus
  source 1..3739: mol_type = mRNA
ctgagagcagtgtgcgtggagattaggtccgagcgcgcgccccaggaaggcagtccgcgagctccagccgccgccggcagcagcagcctctgcgaacagagccgcggcggaagcgggcgcaggggagcgagcccggccccgccagcccagcccagcccagcccggcccagcccagcccagcccagccctgccgacaccctccccacacccgggcagcagccgccgtcgcctggagagaaggtggaggaagaaatccagctcctagcacgcacgcaccatcatggaccattatgattcccagcagaccaacgactacatgcagcctgaagaggactgggatcgggacctgctcctggatccggcctgggagaagcagcagaggaagacgttcacagcctggtgcaactcccacctgcgcaaagcggggacacagatcgagaacatcgaagaggacttccgagatggcctgaagctcatgctgctcctggaggtcatctcaggtgaacgcttggccaagccagagagaggcaagatgagagtgcacaagatctccaacgtcaacaaggccttggatttcatagccagcaaaggggtcaagctggtgtccatcggagctgaagaaattgtggatgggaatgtgaagatgaccctgggcatgatctggaccatcattctccgctttgccatccaggacatctccgtggaagagacttcagctaaagaagggttactcctgtggtgtcagaggaagacagccccatataaaaatgtcaacatccaaaacttccacatcagttggaaagatggtcttggtttctgtgccttgatccaccgacaccggcccgagttgattgactatggaaagcttcgaaaggatgatccactcacaaacctgaacacggcctttgatgtggcagagaggttcctggacatccctaagatgctggatgctgaagacatcgtcggaactgcccggccagatgagaaagccattatgacttatgtgtctagcttctatcatgccttctctggagcccagaaggcagaaacagcagccaatcgcatctgcaaagtgttggcggtcaatcaagagaacgagcagcttatggaagactatgagaagctggccagtgatctgttggagtggatccgccgcaccatcccatggctggagaatcgggtgcctgagaacaccatgcatgccatgcagcagaagctggaggacttccgagactatagacgcctgcacaagccgcccaaggtgcaggagaagtgccagctggagatcaactttaacacgctgcagaccaagctgcggctcagcaaccggcctgccttcatgccctccgagggcaggatggtctcggatatcaacaatgcctggggctgcctggaacaggccgagaagggctacgaggagtggctgctgaatgagatccggaggctagagaggttggaccacctggcagagaagttccggcagaaggcctccatccatgaagcctggacggatggcaaagaagccatgcttcggcagaaggattatgagacagccaccctgtccgagatcaaggccttgctcaagaagcacgaggcttttgagagtgatctggctgcccaccaggaccgcgtggagcagattgcggccattgcccaggagcttaatgagctggactactatgactcgccaagtgtcaacgctcgttgccaaaagatctgtgaccagtgggacaatctaggggctctcactcagaagcggagggaagctttagagcgaacagagaagctgctggaaaccatcgaccagctctacttagagtacgccaagagggctgcacccttcaacaattggatggagggagccatggaggacctgcaggacaccttcatcgtacataccatcgaggagatccagggactgaccacagcccatgagcagttcaaggccaccctcccggatgcagacaaggagcgcctcgccattctgggcatccacaatgaagtgtccaagatcgtccagacctatcatgtcaacatggcaggcaccaacccctatacaaccatcacgcctcaggagatcaatggcaaatgggaccatgtacggcagctagtgccccggagggaccaggctctcacagaggaacatgcaaggcagcagcacaatgagaggctacgcaaacagtttggcgcacaagccaatgtcattgggccctggatccagaccaagatggaggagattgggaggatttccattgagatgcacggcaccctggaggaccaactcagccacctgcggcagtatgagaagagcatcgtcaattacaagccaaagattgaccagctggagtgcgaccaccagctcatccaggaggcacttatctttgacaataagcacaccaactacaacatggagcatatccgtgtgggctgggagcagctgctcaccaccattgccaggaccatcaatgaagtggagaaccagatcttgacccgggatgccaaaggcatcagccaggaacagatgaacgaattccgcgcctctttcaaccactttgaccgggatcactccggcacgttgggtcccgaagagttcaaagcctgcctcatcagcttgggttatgatattggcaacgacccccagggagaggcagagtttgcccgaatcatgagcattgtagaccccaaccgcttgggggtagtgacgttccaggccttcatcgactttatgtcccgagagacggcagatactgatactgcagatcaggtcatggcttccttcaagatcctggctggagataagaattacatcacagaggatgagctgcgccgtgagctgccccctgaccaagccgagtactgcatcgcaagaatggccccctacgcgggccccgactccgtgcccggtgctttagactacatgtccttctccacggcactgtatggcgagagcgacctctaacccgcccctgcctccctccaccacccccccacccccggccttctgcgcgtgcgccccaccctgctcctcctcctcctccgcccagtctggctggctgggttcattattttcagctcagccaccaagggggcagagctccgacccaggcggcatcaagcctccctgcccacaaagtgacagtttacaaaattattttctgcaaaaaaggataaaaaaaaagttacgttaaaaaaaaattttaaaaagaaaaaaaaaaacaaacaaaaaactacatattttattataaaaaaaaaagtatttttttctccaccagtcttaaatggaaaagaggaaaaattaactatttgcaccgaaatgtttttgttgtgacataggaaaataaccaagcacaaagttatattccattctttttactgaagttttttcctcctacctgttctgtctgctgtattcatttctctaatttcatgcccacttgggggtggggtaggtgggtactctcatcagaggcacattggtgcatgtgtgtttgctagctcacttgtccatgagaatattttatgatattaaagaaaaccttttgaaatggctgcttttcaaagaagataattcatggcttctcatttttcagtctcttcaaaagtgtggctggcctgttttatgactgcagagttgttatgttttttaattttgaatattgcctattaaagataggacaaacttggagattatgatgttgcttggcacagactgtattaaaacaacactcccgtgacatta